SEQUENCE LISTING (212 sequences)

SEQ ID NO: 1 - protein, 133 aa, Homo sapiens [sequence omitted]

SEQ ID NO: 2 - protein, 16 aa, synthetic construct
  source 1..16: note = Human IL2 IL2RB BL2
RPRDLISNINVIVLEL

SEQ ID NO: 3 - skipped

SEQ ID NO: 4 - protein, 16 aa, synthetic construct
  source 1..16: note = IL2RB BL2 OF EP564
RFKALIIEINFIVQLL

SEQ ID NO: 5 - protein, 16 aa, synthetic construct
  source 1..16: note = IL2RB BL2 OF EP565
RSRQLISNMNGIILKL

SEQ ID NO: 6 - protein, 16 aa, synthetic construct
  source 1..16: note = IL2RB BL2 OF EP566
RLTHLRNVIGVILVQL

SEQ ID NO: 7 - protein, 16 aa, synthetic construct
  source 1..16: note = IL2RB BL2 OF EP567
LSLREPIGNIVTSVRE

SEQ ID NO: 8 - protein, 16 aa, synthetic construct
  source 1..16: note = IL2RB BL2 OF EP573
NRTDLVGDVNATIKAL

SEQ ID NO: 9 - protein, 16 aa, synthetic construct
  source 1..16: note = IL2RB BL2 OF EP575
RNKGILGDISNIVLAL

SEQ ID NO: 10 - protein, 16 aa, synthetic construct
  source 1..16: note = IL2RB BL2 OF EP581
RSREVVSRIDAIILEL

SEQ ID NO: 11 - protein, 16 aa, synthetic construct
  source 1..16: note = IL2RB BL2 OF EP658
RPRGLISDISNIVLAL

SEQ ID NO: 12 - protein, 16 aa, synthetic construct
  source 1..16: note = IL2RB BL2 OF EP659
RPRGLIGNISNIVLAL

SEQ ID NO: 13 - protein, 16 aa, synthetic construct
  source 1..16: note = IL2RB BL2 OF EP660
RPRGLIGDINNIVLAL

SEQ ID NO: 14 - protein, 16 aa, synthetic construct
  source 1..16: note = IL2RB BL2 OF EP661
RPKGLISNISNIVLAL

SEQ ID NO: 15 - protein, 16 aa, synthetic construct
  source 1..16: note = IL2RB BL2 OF EP662
RPKGLISDINNIVLAL

SEQ ID NO: 16 - protein, 16 aa, synthetic construct
  source 1..16: note = IL2RB BL2 OF EP663
RPKGLIGNINNIVLAL

SEQ ID NO: 17 - protein, 16 aa, synthetic construct
  source 1..16: note = IL2RB BL2 OF EP664
RNRGLISNISNIVLAL

SEQ ID NO: 18 - protein, 16 aa, synthetic construct
  source 1..16: note = IL2RB BL2 OF EP665
RNRGLISDINNIVLAL

SEQ ID NO: 19 - protein, 16 aa, synthetic construct
  source 1..16: note = IL2RB BL2 OF EP666
RNRGLIGNINNIVLAL

SEQ ID NO: 20 - protein, 16 aa, synthetic construct
  source 1..16: note = IL2RB BL2 OF EP667
RNKGLISNINNIVLAL

SEQ ID NO: 21 - protein, 16 aa, synthetic construct
  source 1..16: note = IL2RB BL2 OF EP668
RPRGLIGDISNIVLAL

SEQ ID NO: 22 - protein, 16 aa, synthetic construct
  source 1..16: note = IL2RB BL2 OF EP669
RPKGLISDISNIVLAL

SEQ ID NO: 23 - protein, 16 aa, synthetic construct
  source 1..16: note = IL2RB BL2 OF EP670
RPKGLIGNISNIVLAL

SEQ ID NO: 24 - protein, 16 aa, synthetic construct
  source 1..16: note = IL2RB BL2 OF EP671
RPKGLIGDINNIVLAL

SEQ ID NO: 25 - protein, 16 aa, synthetic construct
  source 1..16: note = IL2RB BL2 OF EP672
RNRGLISDISNIVLAL

SEQ ID NO: 26 - protein, 16 aa, synthetic construct
  source 1..16: note = IL2RB BL2 OF EP673
RNRGLIGNISNIVLAL

SEQ ID NO: 27 - protein, 16 aa, synthetic construct
  source 1..16: note = IL2RB BL2 OF EP674
RNRGLIGDINNIVLAL

SEQ ID NO: 28 - protein, 16 aa, synthetic construct
  source 1..16: note = IL2RB BL2 OF EP675
RNKGLISNISNIVLAL

SEQ ID NO: 29 - protein, 16 aa, synthetic construct
  source 1..16: note = IL2RB BL2 OF EP676
RNKGLISDINNIVLAL

SEQ ID NO: 30 - protein, 16 aa, synthetic construct
  source 1..16: note = IL2RB BL2 OF EP677
RNKGLIGNINNIVLAL

SEQ ID NO: 31 - protein, 16 aa, synthetic construct
  source 1..16: note = IL2RB BL2 OF EP722
RPRDLISDISNIVLAL

SEQ ID NO: 32 - protein, 16 aa, synthetic construct
  source 1..16: note = IL2RB BL2 OF EP723
RPRGLISDINNIVLAL

SEQ ID NO: 33 - protein, 16 aa, synthetic construct
  source 1..16: note = IL2RB BL2 OF EP724
RPRGLISDISVIVLAL

SEQ ID NO: 34 - protein, 16 aa, synthetic construct
  source 1..16: note = IL2RB BL2 OF EP725
RPRGLISDISNIVLEL

SEQ ID NO: 35 - protein, 16 aa, synthetic construct
  source 1..16: note = IL2RB BL2 OF EP726
RPRDLISDINNIVLAL

SEQ ID NO: 36 - protein, 16 aa, synthetic construct
  source 1..16: note = IL2RB BL2 OF EP727
RPRDLISDISVIVLAL

SEQ ID NO: 37 - protein, 16 aa, synthetic construct
  source 1..16: note = IL2RB BL2 OF EP728
RPRDLISDISNIVLEL

SEQ ID NO: 38 - protein, 16 aa, synthetic construct
  source 1..16: note = IL2RB BL2 OF EP729
RPRGLISDINVIVLAL

SEQ ID NO: 39 - protein, 16 aa, synthetic construct
  source 1..16: note = IL2RB BL2 OF EP730
RPRGLISDINNIVLEL

SEQ ID NO: 40 - protein, 16 aa, synthetic construct
  source 1..16: note = IL2RB BL2 OF EP731
RPRGLISDISVIVLEL

SEQ ID NO: 41 - protein, 16 aa, synthetic construct
  source 1..16: note = IL2RB BL2 OF EP732
RPKDLISNISNIVLAL

SEQ ID NO: 42 - protein, 16 aa, synthetic construct
  source 1..16: note = IL2RB BL2 OF EP733
RPKGLISNINNIVLAL

SEQ ID NO: 43 - protein, 16 aa, synthetic construct
  source 1..16: note = IL2RB BL2 OF EP734
RPKGLISNISVIVLAL

SEQ ID NO: 44 - protein, 16 aa, synthetic construct
  source 1..16: note = IL2RB BL2 OF EP735
RPKGLISNISNIVLEL

SEQ ID NO: 45 - protein, 133 aa, synthetic construct
  VARIANT 81: note = X = R, S, L, N, F, K, OR T
  VARIANT 82: note = X = A, F, S, L, R, T, I, H, P, OR N
  VARIANT 83: note = X = K, R, T, S, I, OR P
  VARIANT 84: note = X = A G, D, R, A, Q, H, N, Y, OR E
  VARIANT 85: note = X = I, P, T, S, K, F, V, OR L
  VARIANT 86: note = X = I, R, V, M, T, OR L
  VARIANT 87: note = X = A, R, M, I, S, N, G, OR S
  VARIANT 88: note = X = E, N, H, T, K, Y, S, L, V, D, OR R
  VARIANT 89: note = X = I, V, A, T, L, T, OR M
  VARIANT 90: note = X = N, G, V, Y, I, W, R, K, Q, A, D, S, OR D
  VARIANT 91: note = X = F, G, V, N, T, I, R, E, OR A
  VARIANT 92: note = X = I, S, R, V, P, G, T, L, M, F, OR Y
  VARIANT 93: note = X = V, I, F, D, P, H, A, V, OR L
  VARIANT 94: note = X = L, Q, R, E, P, K, H, W, F, OR V
  VARIANT 95: note = X = A, E, L, K, V, D, Y, R, OR Q
  VARIANT 96: note = X = L OR I
APASSSTKKTQLQLEHLLLDLQMILNGINNYKNPLLTDMLTRKFYMPKKATELKHLQCLEEELKPLEEVLNLAQSKNFHLXXXXXXXXXXXXXXXXKGSETTFMCEYADETATIVEFLNRWITFSQSIISTLT

SEQ ID NO: 46 - protein, 133 aa, synthetic construct
  source 1..133: note = EP564
APASSSTKKTQLQLEHLLLDLQMILNGINNYKNPLLTDMLTRKFYMPKKATELKHLQCLEEELKPLEEVLNLAQSKNFHLRFKALIIEINFIVQLLKGSETTFMCEYADETATIVEFLNRWITFSQSIISTLT

SEQ ID NO: 47 - protein, 133 aa, synthetic construct
  source 1..133: note = EP565
APASSSTKKTQLQLEHLLLDLQMILNGINNYKNPLLTDMLTRKFYMPKKATELKHLQCLEEELKPLEEVLNLAQSKNFHLRSRQLISNMNGIILKLKGSETTFMCEYADETATIVEFLNRWITFSQSIISTLT

SEQ ID NO: 48 - protein, 133 aa, synthetic construct
  source 1..133: note = EP566
APASSSTKKTQLQLEHLLLDLQMILNGINNYKNPLLTDMLTRKFYMPKKATELKHLQCLEEELKPLEEVLNLAQSKNFHLRLTHLRNVIGVILVQLKGSETTFMCEYADETATIVEFLNRWITFSQSIISTLT

SEQ ID NO: 49 - protein, 133 aa, synthetic construct
  source 1..133: note = EP567
APASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 50 - protein, 133 aa, synthetic construct
  source 1..133: note = EP568, 2021EP16-H06 WITH K35L/R38D/F42R MUTATIONS
APASSSTKKTQLQLEHLLLDLQMILNGINNYKNPLLTDMLTRKFYMPKKATELKHLQCLEEELKPLEEVLNLAQSKNFHLLAKRTVSNMYVRVEELKGSETTFMCEYADETATIVEFLNRWITFSQSIISTLT

SEQ ID NO: 51 - protein, 133 aa, synthetic construct
  source 1..133: note = EP569
APASSSTKKTQLQLEHLLLDLQMILNGINNYKNPLLTDMLTRKFYMPKKATELKHLQCLEEELKPLEEVLNLAQSKNFHLRRKESINHMIVVFPVLKGSETTFMCEYADETATIVEFLNRWITFSQSIISTLT

SEQ ID NO: 52 - protein, 133 aa, synthetic construct
  source 1..133: note = EP570
APASSSTKKTQLQLEHLLLDLQMILNGINNYKNPLLTDMLTRKFYMPKKATELKHLQCLEEELKPLEEVLNLAQSKNFHLSSSGLLGDVWVIVLDLKGSETTFMCEYADETATIVEFLNRWITFSQSIISTLT

SEQ ID NO: 53 - protein, 133 aa, synthetic construct
  source 1..133: note = EP571
APASSSTKKTQLQLEHLLLDLQMILNGINNYKNPLLTDMLTRKFYMPKKATELKHLQCLEEELKPLEEVLNLAQSKNFHLRPSELMATMRVPVQALKGSETTFMCEYADETATIVEFLNRWITFSQSIISTLT

SEQ ID NO: 54 - protein, 133 aa, synthetic construct
  source 1..133: note = EP572
APASSSTKKTQLQLEHLLLDLQMILNGINNYKNPLLTDMLTRKFYMPKKATELKHLQCLEEELKPLEEVLNLAQSKNFHLRARDIVRNAKVGVQELKGSETTFMCEYADETATIVEFLNRWITFSQSIISTLT

SEQ ID NO: 55 - protein, 133 aa, synthetic construct
  source 1..133: note = EP573
APASSSTKKTQLQLEHLLLDLQMILNGINNYKNPLLTDMLTRKFYMPKKATELKHLQCLEEELKPLEEVLNLAQSKNFHLNRTDLVGDVNATIKALKGSETTFMCEYADETATIVEFLNRWITFSQSIISTLT

SEQ ID NO: 56 - protein, 133 aa, synthetic construct
  source 1..133: note = EP574
APASSSTKKTQLQLEHLLLDLQMILNGINNYKNPLLTDMLTRKFYMPKKATELKHLQCLEEELKPLEEVLNLAQSKNFHLFTKDLTSTMQILVLQLKGSETTFMCEYADETATIVEFLNRWITFSQSIISTLT

SEQ ID NO: 57 - protein, 133 aa, synthetic construct
  source 1..133: note = EP575
APASSSTKKTQLQLEHLLLDLQMILNGINNYKNPLLTDMLTRKFYMPKKATELKHLQCLEEELKPLEEVLNLAQSKNFHLRNKGILGDISNIVLALKGSETTFMCEYADETATIVEFLNRWITFSQSIISTLT

SEQ ID NO: 58 - protein, 133 aa, synthetic construct
  source 1..133: note = EP576
APASSSTKKTQLQLEHLLLDLQMILNGINNYKNPLLTDMLTRKFYMPKKATELKHLQCLEEELKPLEEVLNLAQSKNFHLLPRNKISKTKVMVHELKGSETTFMCEYADETATIVEFLNRWITFSQSIISTLT

SEQ ID NO: 59 - protein, 133 aa, synthetic construct
  source 1..133: note = EP577
APASSSTKKTQLQLEHLLLDLQMILNGINNYKNPLLTDMLTRKFYMPKKATELKHLQCLEEELKPLEEVLNLAQSKNFHLKAIAFISDLKVRVLDLKGSETTFMCEYADETATIVEFLNRWITFSQSIISTLT

SEQ ID NO: 60 - protein, 133 aa, synthetic construct
  source 1..133: note = EP578
APASSSTKKTQLQLEHLLLDLQMILNGINNYKNPLLTDMLTRKFYMPKKATELKHLQCLEEELKPLEEVLNLAQSKNFHLTTRDVVMTMARIDLELKGSETTFMCEYADETATIVEFLNRWITFSQSIISTLT

SEQ ID NO: 61 - protein, 133 aa, synthetic construct
  source 1..133: note = EP579
APASSSTKKTQLQLEHLLLDLQMILNGINNYKNPLLTDMLTRKFYMPKKATELKHLQCLEEELKPLEEVLNLAQSKNFHLRPSYIVSYTNVFPLVLKGSETTFMCEYADETATIVEFLNRWITFSQSIISTLT

SEQ ID NO: 62 - protein, 133 aa, synthetic construct
  source 1..133: note = EP580
APASSSTKKTQLQLEHLLLDLQMILNGINNYKNPLLTDMLTRKFYMPKKATELKHLQCLEEELKPLEEVLNLAQSKNFHLRIRDLLNKINELHRYIKGSETTFMCEYADETATIVEFLNRWITFSQSIISTLT

SEQ ID NO: 63 - protein, 133 aa, synthetic construct
  source 1..133: note = EP581
APASSSTKKTQLQLEHLLLDLQMILNGINNYKNPLLTDMLTRKFYMPKKATELKHLQCLEEELKPLEEVLNLAQSKNFHLRSREVVSRIDAIILELKGSETTFMCEYADETATIVEFLNRWITFSQSIISTLT

SEQ ID NO: 64 - protein, 133 aa, synthetic construct
  source 1..133: note = EP582
APASSSTKKTQLQLEHLLLDLQMILNGINNYKNPLLTDMLTRKFYMPKKATELKHLQCLEEELKPLEEVLNLAQSKNFHLTHRDVISDVNVLALELKGSETTFMCEYADETATIVEFLNRWITFSQSIISTLT

SEQ ID NO: 65 - protein, 133 aa, synthetic construct
  source 1..133: note = EP583
APASSSTKKTQLQLEHLLLDLQMILNGINNYKNPLLTDMLTRKFYMPKKATELKHLQCLEEELKPLEEVLNLAQSKNFHLSAIDLMSSVKVVVWKLKGSETTFMCEYADETATIVEFLNRWITFSQSIISTLT

SEQ ID NO: 66 - protein, 133 aa, synthetic construct
  source 1..133: note = EP584
APASSSTKKTQLQLEHLLLDLQMILNGINNYKNPLLTDMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 67 - protein, 133 aa, synthetic construct
  source 1..133: note = EP585
APASSSTKKTQLQLEHLLLDLQMILNGINNYKNPLLTDMLTRKFYMPKKATELKHLQCLEEELKPLEEVLNLAQSKNFHLSPPDLIANMAVYFVELKGSETTFMCEYADETATIVEFLNRWITFSQSIISTLT

SEQ ID NO: 68 - protein, 133 aa, synthetic construct
  source 1..133: note = EP586
APASSSTKKTQLQLEHLLLDLQMILNGINNYKNPLLTDMLTRKFYMPKKATELKHLQCLEEELKPLEEVLNLAQSKNFHLRPRDSRRLISVIVFRLKGSETTFMCEYADETATIVEFLNRWITFSQSIISTLT

SEQ ID NO: 69 - protein, 133 aa, synthetic construct
  source 1..133: note = EP658
APASSSTKKTQLQLEHLLLDLQMILNGINNYKNPLLTDMLTRKFYMPKKATELKHLQCLEEELKPLEEVLNLAQSKNFHLRPRGLISDISNIVLALKGSETTFMCEYADETATIVEFLNRWITFSQSIISTLT

SEQ ID NO: 70 - protein, 133 aa, synthetic construct
  source 1..133: note = EP659
APASSSTKKTQLQLEHLLLDLQMILNGINNYKNPLLTDMLTRKFYMPKKATELKHLQCLEEELKPLEEVLNLAQSKNFHLRPRGLIGNISNIVLALKGSETTFMCEYADETATIVEFLNRWITFSQSIISTLT

SEQ ID NO: 71 - protein, 133 aa, synthetic construct
  source 1..133: note = EP660
APASSSTKKTQLQLEHLLLDLQMILNGINNYKNPLLTDMLTRKFYMPKKATELKHLQCLEEELKPLEEVLNLAQSKNFHLRPRGLIGDINNIVLALKGSETTFMCEYADETATIVEFLNRWITFSQSIISTLT

SEQ ID NO: 72 - protein, 133 aa, synthetic construct
  source 1..133: note = EP661
APASSSTKKTQLQLEHLLLDLQMILNGINNYKNPLLTDMLTRKFYMPKKATELKHLQCLEEELKPLEEVLNLAQSKNFHLRPKGLISNISNIVLALKGSETTFMCEYADETATIVEFLNRWITFSQSIISTLT

SEQ ID NO: 73 - protein, 133 aa, synthetic construct
  source 1..133: note = EP662
APASSSTKKTQLQLEHLLLDLQMILNGINNYKNPLLTDMLTRKFYMPKKATELKHLQCLEEELKPLEEVLNLAQSKNFHLRPKGLISDINNIVLALKGSETTFMCEYADETATIVEFLNRWITFSQSIISTLT

SEQ ID NO: 74 - protein, 133 aa, synthetic construct
  source 1..133: note = EP663
APASSSTKKTQLQLEHLLLDLQMILNGINNYKNPLLTDMLTRKFYMPKKATELKHLQCLEEELKPLEEVLNLAQSKNFHLRPKGLIGNINNIVLALKGSETTFMCEYADETATIVEFLNRWITFSQSIISTLT

SEQ ID NO: 75 - protein, 133 aa, synthetic construct
  source 1..133: note = EP664
APASSSTKKTQLQLEHLLLDLQMILNGINNYKNPLLTDMLTRKFYMPKKATELKHLQCLEEELKPLEEVLNLAQSKNFHLRNRGLISNISNIVLALKGSETTFMCEYADETATIVEFLNRWITFSQSIISTLT

SEQ ID NO: 76 - protein, 133 aa, synthetic construct
  source 1..133: note = EP665
APASSSTKKTQLQLEHLLLDLQMILNGINNYKNPLLTDMLTRKFYMPKKATELKHLQCLEEELKPLEEVLNLAQSKNFHLRNRGLISDINNIVLALKGSETTFMCEYADETATIVEFLNRWITFSQSIISTLT

SEQ ID NO: 77 - protein, 133 aa, synthetic construct
  source 1..133: note = EP666
APASSSTKKTQLQLEHLLLDLQMILNGINNYKNPLLTDMLTRKFYMPKKATELKHLQCLEEELKPLEEVLNLAQSKNFHLRNRGLIGNINNIVLALKGSETTFMCEYADETATIVEFLNRWITFSQSIISTLT

SEQ ID NO: 78 - protein, 133 aa, synthetic construct
  source 1..133: note = EP667
APASSSTKKTQLQLEHLLLDLQMILNGINNYKNPLLTDMLTRKFYMPKKATELKHLQCLEEELKPLEEVLNLAQSKNFHLRNKGLISNINNIVLALKGSETTFMCEYADETATIVEFLNRWITFSQSIISTLT

SEQ ID NO: 79 - protein, 133 aa, synthetic construct
  source 1..133: note = EP668
APASSSTKKTQLQLEHLLLDLQMILNGINNYKNPLLTDMLTRKFYMPKKATELKHLQCLEEELKPLEEVLNLAQSKNFHLRPRGLIGDISNIVLALKGSETTFMCEYADETATIVEFLNRWITFSQSIISTLT

SEQ ID NO: 80 - protein, 133 aa, synthetic construct
  source 1..133: note = EP669
APASSSTKKTQLQLEHLLLDLQMILNGINNYKNPLLTDMLTRKFYMPKKATELKHLQCLEEELKPLEEVLNLAQSKNFHLRPKGLISDISNIVLALKGSETTFMCEYADETATIVEFLNRWITFSQSIISTLT

SEQ ID NO: 81 - protein, 133 aa, synthetic construct
  source 1..133: note = EP670
APASSSTKKTQLQLEHLLLDLQMILNGINNYKNPLLTDMLTRKFYMPKKATELKHLQCLEEELKPLEEVLNLAQSKNFHLRPKGLIGNISNIVLALKGSETTFMCEYADETATIVEFLNRWITFSQSIISTLT

SEQ ID NO: 82 - protein, 133 aa, synthetic construct
  source 1..133: note = EP671
APASSSTKKTQLQLEHLLLDLQMILNGINNYKNPLLTDMLTRKFYMPKKATELKHLQCLEEELKPLEEVLNLAQSKNFHLRPKGLIGDINNIVLALKGSETTFMCEYADETATIVEFLNRWITFSQSIISTLT

SEQ ID NO: 83 - protein, 133 aa, synthetic construct
  source 1..133: note = EP672
APASSSTKKTQLQLEHLLLDLQMILNGINNYKNPLLTDMLTRKFYMPKKATELKHLQCLEEELKPLEEVLNLAQSKNFHLRNRGLISDISNIVLALKGSETTFMCEYADETATIVEFLNRWITFSQSIISTLT

SEQ ID NO: 84 - protein, 133 aa, synthetic construct
  source 1..133: note = EP673
APASSSTKKTQLQLEHLLLDLQMILNGINNYKNPLLTDMLTRKFYMPKKATELKHLQCLEEELKPLEEVLNLAQSKNFHLRNRGLIGNISNIVLALKGSETTFMCEYADETATIVEFLNRWITFSQSIISTLT

SEQ ID NO: 85 - protein, 141 aa, synthetic construct
  source 1..141: note = EP674
APASSSTKKTQLQLEHLLLDLQMILNGINNYKNPLLTDMLTRKFYMPKKATELKHLQCLEEELKPLEEVLNLAQSKNFHLRNRGLIGDINNIVLALKGSETTFMCEYADETATIVEFLNRWITFSQSIISTLTGSHHHHHH

SEQ ID NO: 86 - protein, 133 aa, synthetic construct
  source 1..133: note = EP675
APASSSTKKTQLQLEHLLLDLQMILNGINNYKNPLLTDMLTRKFYMPKKATELKHLQCLEEELKPLEEVLNLAQSKNFHLRNKGLISNISNIVLALKGSETTFMCEYADETATIVEFLNRWITFSQSIISTLT

SEQ ID NO: 87 - protein, 133 aa, synthetic construct
  source 1..133: note = EP676
APASSSTKKTQLQLEHLLLDLQMILNGINNYKNPLLTDMLTRKFYMPKKATELKHLQCLEEELKPLEEVLNLAQSKNFHLRNKGLISDINNIVLALKGSETTFMCEYADETATIVEFLNRWITFSQSIISTLT

SEQ ID NO: 88 - protein, 133 aa, synthetic construct
  source 1..133: note = EP677
APASSSTKKTQLQLEHLLLDLQMILNGINNYKNPLLTDMLTRKFYMPKKATELKHLQCLEEELKPLEEVLNLAQSKNFHLRNKGLIGNINNIVLALKGSETTFMCEYADETATIVEFLNRWITFSQSIISTLT

SEQ ID NO: 89 - protein, 133 aa, synthetic construct
  source 1..133: note = EP722
APASSSTKKTQLQLEHLLLDLQMILNGINNYKNPLLTDMLTRKFYMPKKATELKHLQCLEEELKPLEEVLNLAQSKNFHLRPRDLISDISNIVLALKGSETTFMCEYADETATIVEFLNRWITFSQSIISTLT

SEQ ID NO: 90 - protein, 133 aa, synthetic construct
  source 1..133: note = EP723
APASSSTKKTQLQLEHLLLDLQMILNGINNYKNPLLTDMLTRKFYMPKKATELKHLQCLEEELKPLEEVLNLAQSKNFHLRPRGLISDINNIVLALKGSETTFMCEYADETATIVEFLNRWITFSQSIISTLT

SEQ ID NO: 91 - protein, 133 aa, synthetic construct
  source 1..133: note = EP724
APASSSTKKTQLQLEHLLLDLQMILNGINNYKNPLLTDMLTRKFYMPKKATELKHLQCLEEELKPLEEVLNLAQSKNFHLRPRGLISDISVIVLALKGSETTFMCEYADETATIVEFLNRWITFSQSIISTLT

SEQ ID NO: 92 - protein, 133 aa, synthetic construct
  source 1..133: note = EP725
APASSSTKKTQLQLEHLLLDLQMILNGINNYKNPLLTDMLTRKFYMPKKATELKHLQCLEEELKPLEEVLNLAQSKNFHLRPRGLISDISNIVLELKGSETTFMCEYADETATIVEFLNRWITFSQSIISTLT

SEQ ID NO: 93 - protein, 133 aa, synthetic construct
  source 1..133: note = EP726
APASSSTKKTQLQLEHLLLDLQMILNGINNYKNPLLTDMLTRKFYMPKKATELKHLQCLEEELKPLEEVLNLAQSKNFHLRPRDLISDINNIVLALKGSETTFMCEYADETATIVEFLNRWITFSQSIISTLT

SEQ ID NO: 94 - protein, 133 aa, synthetic construct
  source 1..133: note = EP727
APASSSTKKTQLQLEHLLLDLQMILNGINNYKNPLLTDMLTRKFYMPKKATELKHLQCLEEELKPLEEVLNLAQSKNFHLRPRDLISDISVIVLALKGSETTFMCEYADETATIVEFLNRWITFSQSIISTLT

SEQ ID NO: 95 - protein, 133 aa, synthetic construct
  source 1..133: note = EP728
APASSSTKKTQLQLEHLLLDLQMILNGINNYKNPLLTDMLTRKFYMPKKATELKHLQCLEEELKPLEEVLNLAQSKNFHLRPRDLISDISNIVLELKGSETTFMCEYADETATIVEFLNRWITFSQSIISTLT

SEQ ID NO: 96 - protein, 133 aa, synthetic construct
  source 1..133: note = EP729
APASSSTKKTQLQLEHLLLDLQMILNGINNYKNPLLTDMLTRKFYMPKKATELKHLQCLEEELKPLEEVLNLAQSKNFHLRPRGLISDINVIVLALKGSETTFMCEYADETATIVEFLNRWITFSQSIISTLT

SEQ ID NO: 97 - protein, 133 aa, synthetic construct
  source 1..133: note = EP730
APASSSTKKTQLQLEHLLLDLQMILNGINNYKNPLLTDMLTRKFYMPKKATELKHLQCLEEELKPLEEVLNLAQSKNFHLRPRGLISDINNIVLELKGSETTFMCEYADETATIVEFLNRWITFSQSIISTLT

SEQ ID NO: 98 - protein, 133 aa, synthetic construct
  source 1..133: note = EP731
APASSSTKKTQLQLEHLLLDLQMILNGINNYKNPLLTDMLTRKFYMPKKATELKHLQCLEEELKPLEEVLNLAQSKNFHLRPRGLISDISVIVLELKGSETTFMCEYADETATIVEFLNRWITFSQSIISTLT

SEQ ID NO: 99 - protein, 133 aa, synthetic construct
  source 1..133: note = EP732
APASSSTKKTQLQLEHLLLDLQMILNGINNYKNPLLTDMLTRKFYMPKKATELKHLQCLEEELKPLEEVLNLAQSKNFHLRPKDLISNISNIVLALKGSETTFMCEYADETATIVEFLNRWITFSQSIISTLT

SEQ ID NO: 100 - protein, 133 aa, synthetic construct
  source 1..133: note = EP733
APASSSTKKTQLQLEHLLLDLQMILNGINNYKNPLLTDMLTRKFYMPKKATELKHLQCLEEELKPLEEVLNLAQSKNFHLRPKGLISNINNIVLALKGSETTFMCEYADETATIVEFLNRWITFSQSIISTLT

SEQ ID NO: 101 - protein, 133 aa, synthetic construct
  source 1..133: note = EP734
APASSSTKKTQLQLEHLLLDLQMILNGINNYKNPLLTDMLTRKFYMPKKATELKHLQCLEEELKPLEEVLNLAQSKNFHLRPKGLISNISVIVLALKGSETTFMCEYADETATIVEFLNRWITFSQSIISTLT

SEQ ID NO: 102 - protein, 133 aa, synthetic construct
  source 1..133: note = EP735
APASSSTKKTQLQLEHLLLDLQMILNGINNYKNPLLTDMLTRKFYMPKKATELKHLQCLEEELKPLEEVLNLAQSKNFHLRPKGLISNISNIVLELKGSETTFMCEYADETATIVEFLNRWITFSQSIISTLT

SEQ ID NO: 103 - protein, 216 aa, synthetic construct
  source 1..216: note = EP205
QSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 104 - protein, 451 aa, synthetic construct
  source 1..451: note = EP362
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 105 - protein, 603 aa, synthetic construct
  source 1..603: note = EP363
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 106 - protein, 603 aa, synthetic construct
  source 1..603: note = EP642
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 107 - protein, 604 aa, synthetic construct
  source 1..604: note = EP643
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRRNKGILGDISNIVLALKGSETTFMCEYADETATIVEFLNRWITFSQSIISTLT

SEQ ID NO: 108 - protein, 603 aa, synthetic construct
  source 1..603: note = EP736
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRGLISDISNIVLALKGSETTFMCEYADETATIVEFLNRWITFSQSIISTLT

SEQ ID NO: 109 - protein, 603 aa, synthetic construct
  source 1..603: note = EP737
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISNISNIVLALKGSETTFMCEYADETATIVEFLNRWITFSQSIISTLT

SEQ ID NO: 110 - protein, 603 aa, synthetic construct
  source 1..603: note = EP741
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRSREVVSRIDAIILELKGSETTFMCEYADETATIVEFLNRWITFSQSIISTLT

SEQ ID NO: 111 - protein, 603 aa, synthetic construct
  source 1..603: note = EP742
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISDISNIVLALKGSETTFMCEYADETATIVEFLNRWITFSQSIISTLT

SEQ ID NO: 112 - protein, 603 aa, synthetic construct
  source 1..603: note = EP743
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRGLIGDINNIVLALKGSETTFMCEYADETATIVEFLNRWITFSQSIISTLT

SEQ ID NO: 113 - protein, 603 aa, synthetic construct
  source 1..603: note = EP744
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRNKGLISDINNIVLALKGSETTFMCEYADETATIVEFLNRWITFSQSIISTLT

SEQ ID NO: 114 - protein, 451 aa, synthetic construct
  source 1..451: note = EP325
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 115 - protein, 365 aa, synthetic construct
  source 1..365: note = EP415
APASSSTKKTQLQLEHLLLDLQMILNGINNYKNPLLTDMLTRKFYMPKKATELKHLQCLEEELKPLEEVLNLAQSKNFHLTARDAIDNINVIVQELKGSETTFMCEYADETATIVEFLNRWITFSQSIISTLTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 116 - protein, 450 aa, synthetic construct
  source 1..450: note = EP738
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 117 - protein, 218 aa, synthetic construct
  source 1..218: note = EP740
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 602 aa, synthetic construct
  source 1..602: note = EP785
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRNKGILGDISNIVLALKGSETTFMCEYADETATIVEFLNRWITFSQSIISTLT

SEQ ID NO: 119 - protein, 602 aa, synthetic construct
  source 1..602: note = EP786
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRSREVVSRIDAIILELKGSETTFMCEYADETATIVEFLNRWITFSQSIISTLT

SEQ ID NO: 120 - protein, 602 aa, synthetic construct
  source 1..602: note = EP787
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRGLISDISNIVLALKGSETTFMCEYADETATIVEFLNRWITFSQSIISTLT

SEQ ID NO: 121 - protein, 602 aa, synthetic construct
  source 1..602: note = EP788
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISNISNIVLALKGSETTFMCEYADETATIVEFLNRWITFSQSIISTLT

SEQ ID NO: 122 - protein, 602 aa, synthetic construct
  source 1..602: note = EP789
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISDISNIVLALKGSETTFMCEYADETATIVEFLNRWITFSQSIISTLT

SEQ ID NO: 123 - protein, 227 aa, synthetic construct
  source 1..227: note = EP280
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 124 - protein, 360 aa, synthetic construct
  source 1..360: note = EP826
APASSSTKKTQLQLEHLLLDLQMILNGINNYKNPLLTDMLTRKFYMPKKATELKHLQCLEEELKPLEEVLNLAQSKNFHLRNKGILGDISNIVLALKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 125 - protein, 360 aa, synthetic construct
  source 1..360: note = EP827
APASSSTKKTQLQLEHLLLDLQMILNGINNYKNPLLTDMLTRKFYMPKKATELKHLQCLEEELKPLEEVLNLAQSKNFHLRSREVVSRIDAIILELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 126 - protein, 360 aa, synthetic construct
  source 1..360: note = EP828
APASSSTKKTQLQLEHLLLDLQMILNGINNYKNPLLTDMLTRKFYMPKKATELKHLQCLEEELKPLEEVLNLAQSKNFHLRPRGLISDISNIVLALKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 127 - protein, 360 aa, synthetic construct
  source 1..360: note = EP829
APASSSTKKTQLQLEHLLLDLQMILNGINNYKNPLLTDMLTRKFYMPKKATELKHLQCLEEELKPLEEVLNLAQSKNFHLRPKGLISNISNIVLALKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 128 - protein, 360 aa, synthetic construct
  source 1..360: note = EP830
APASSSTKKTQLQLEHLLLDLQMILNGINNYKNPLLTDMLTRKFYMPKKATELKHLQCLEEELKPLEEVLNLAQSKNFHLRPKGLISDISNIVLALKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 129 - protein, 218 aa, synthetic construct
  source 1..218: note = EP868
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 449 aa, synthetic construct
  source 1..449: note = EP869
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPG

SEQ ID NO: 131 - protein, 602 aa, synthetic construct
  source 1..602: note = EP870
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISNISNIVLALKGSETTFMCEYADETATIVEFLNRWITFSQSIISTLT

SEQ ID NO: 132 - protein, 602 aa, synthetic construct
  source 1..602: note = EP871
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISDISNIVLALKGSETTFMCEYADETATIVEFLNRWITFSQSIISTLT

SEQ ID NO: 133 - protein, 214 aa, synthetic construct
  source 1..214: note = EP872
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 447 aa, synthetic construct
  source 1..447: note = EP873
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPG

SEQ ID NO: 135 - protein, 600 aa, synthetic construct
  source 1..600: note = EP874
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISNISNIVLALKGSETTFMCEYADETATIVEFLNRWITFSQSIISTLT

SEQ ID NO: 136 - protein, 600 aa, synthetic construct
  source 1..600: note = EP875
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISDISNIVLALKGSETTFMCEYADETATIVEFLNRWITFSQSIISTLT

SEQ ID NO: 137 - protein, 227 aa, synthetic construct
  source 1..227: note = IGG1 WT CH2-CH3
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 227 aa, synthetic construct
  source 1..227: note = IGG1 KNOB
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 139 - protein, 121 aa, synthetic construct
  source 1..121: note = 2019EP69-F03 HC
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSS

SEQ ID NO: 140 - protein, 110 aa, synthetic construct
  source 1..110: note = 2019EP69-F03 LC
QSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVL

SEQ ID NO: 141 - protein, 9 aa, synthetic construct
  source 1..9: note = HC CDR1
GTFSSYAIS

SEQ ID NO: 142 - protein, 14 aa, synthetic construct
  source 1..14: note = HC CDR2
WMGGIIPIFGTANY

SEQ ID NO: 143 - protein, 13 aa, synthetic construct
  source 1..13: note = HC CDR3
RDKWYGSAWRGDY

SEQ ID NO: 144 - protein, 10 aa, synthetic construct
  source 1..10: note = LC CDR1
YSNIGSNFVN

SEQ ID NO: 145 - protein, 11 aa, synthetic construct
  source 1..11: note = LC CDR2
LLIHFNNQRPS

SEQ ID NO: 146 - protein, 10 aa, synthetic construct
  source 1..10: note = LC CDR3
STWDDSLKGP

SEQ ID NO: 147 - protein, 133 aa, synthetic construct
  source 1..133: note = 2021EP22-E11
APASSSTKKTQLQLEHLLLDLQMILNGINNYKNPKLTRMLTFKFYMPKKATELKHLQCLEEELKPLEEVLNLAQSKNFHLRFKALIIEINFIVQLLKGSETTFMCEYADETATIVEFLNRWITFSQSIISTLT

SEQ ID NO: 148 - protein, 133 aa, synthetic construct
  source 1..133: note = 2021EP22-F10
APASSSTKKTQLQLEHLLLDLQMILNGINNYKNPKLTRMLTFKFYMPKKATELKHLQCLEEELKPLEEVLNLAQSKNFHLRSRQLISNMNGIILKLKGSETTFMCEYADETATIVEFLNRWITFSQSIISTLT

SEQ ID NO: 149 - protein, 133 aa, synthetic construct
  source 1..133: note = 2021EP22-H09
APASSSTKKTQLQLEHLLLDLQMILNGINNYKNPKLTRMLTFKFYMPKKATELKHLQCLEEELKPLEEVLNLAQSKNFHLRLTHLRNVIGVILVQLKGSETTFMCEYADETATIVEFLNRWITFSQSIISTLT

SEQ ID NO: 150 - protein, 133 aa, synthetic construct
  source 1..133: note = 2021EP21-C04
APASSSTKKTQLQLEHLLLDLQMILNGINNYKNPKLTRMLTFKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 151 - protein, 133 aa, synthetic construct
  source 1..133: note = 2021EP16-H06
APASSSTKKTQLQLEHLLLDLQMILNGINNYKNPKLTRMLTFKFYMPKKATELKHLQCLEEELKPLEEVLNLAQSKNFHLLAKRTVSNMYVRVEELKGSETTFMCEYADETATIVEFLNRWITFSQSIISTLT

SEQ ID NO: 152 - protein, 133 aa, synthetic construct
  source 1..133: note = 2021EP22-E03
APASSSTKKTQLQLEHLLLDLQMILNGINNYKNPKLTRMLTFKFYMPKKATELKHLQCLEEELKPLEEVLNLAQSKNFHLRRKESINHMIVVFPVLKGSETTFMCEYADETATIVEFLNRWITFSQSIISTLT

SEQ ID NO: 153 - protein, 133 aa, synthetic construct
  source 1..133: note = 2021EP21-E03
APASSSTKKTQLQLEHLLLDLQMILNGINNYKNPKLTRMLTFKFYMPKKATELKHLQCLEEELKPLEEVLNLAQSKNFHLSSSGLLGDVWVIVLDLKGSETTFMCEYADETATIVEFLNRWITFSQSIISTLT

SEQ ID NO: 154 - protein, 133 aa, synthetic construct
  source 1..133: note = 2021EP22-H02
APASSSTKKTQLQLEHLLLDLQMILNGINNYKNPKLTRMLTFKFYMPKKATELKHLQCLEEELKPLEEVLNLAQSKNFHLRPSELMATMRVPVQALKGSETTFMCEYADETATIVEFLNRWITFSQSIISTLT

SEQ ID NO: 155 - protein, 133 aa, synthetic construct
  source 1..133: note = 2021EP21-C03
APASSSTKKTQLQLEHLLLDLQMILNGINNYKNPKLTRMLTFKFYMPKKATELKHLQCLEEELKPLEEVLNLAQSKNFHLRARDIVRNAKVGVQELKGSETTFMCEYADETATIVEFLNRWITFSQSIISTLT

SEQ ID NO: 156 - protein, 133 aa, synthetic construct
  source 1..133: note = 2021EP16-E02
APASSSTKKTQLQLEHLLLDLQMILNGINNYKNPKLTRMLTFKFYMPKKATELKHLQCLEEELKPLEEVLNLAQSKNFHLNRTDLVGDVNATIKALKGSETTFMCEYADETATIVEFLNRWITFSQSIISTLT

SEQ ID NO: 157 - protein, 133 aa, synthetic construct
  source 1..133: note = 2021EP22-B04
APASSSTKKTQLQLEHLLLDLQMILNGINNYKNPKLTRMLTFKFYMPKKATELKHLQCLEEELKPLEEVLNLAQSKNFHLFTKDLTSTMQILVLQLKGSETTFMCEYADETATIVEFLNRWITFSQSIISTLT

SEQ ID NO: 158 - protein, 133 aa, synthetic construct
  source 1..133: note = 2021EP16-D09
APASSSTKKTQLQLEHLLLDLQMILNGINNYKNPKLTRMLTFKFYMPKKATELKHLQCLEEELKPLEEVLNLAQSKNFHLRNKGILGDISNIVLALKGSETTFMCEYADETATIVEFLNRWITFSQSIISTLT

SEQ ID NO: 159 - protein, 133 aa, synthetic construct
  source 1..133: note = 2021EP21-C10
APASSSTKKTQLQLEHLLLDLQMILNGINNYKNPKLTRMLTFKFYMPKKATELKHLQCLEEELKPLEEVLNLAQSKNFHLLPRNKISKTKVMVHELKGSETTFMCEYADETATIVEFLNRWITFSQSIISTLT

SEQ ID NO: 160 - protein, 133 aa, synthetic construct
  source 1..133: note = 2021EP16-E05
APASSSTKKTQLQLEHLLLDLQMILNGINNYKNPKLTRMLTFKFYMPKKATELKHLQCLEEELKPLEEVLNLAQSKNFHLKAIAFISDLKVRVLDLKGSETTFMCEYADETATIVEFLNRWITFSQSIISTLT

SEQ ID NO: 161 - protein, 133 aa, synthetic construct
  source 1..133: note = 2021EP16-H02
APASSSTKKTQLQLEHLLLDLQMILNGINNYKNPKLTRMLTFKFYMPKKATELKHLQCLEEELKPLEEVLNLAQSKNFHLTTRDVVMTMARIDLELKGSETTFMCEYADETATIVEFLNRWITFSQSIISTLT

SEQ ID NO: 162 - protein, 133 aa, synthetic construct
  source 1..133: note = 2021EP16-E11
APASSSTKKTQLQLEHLLLDLQMILNGINNYKNPKLTRMLTFKFYMPKKATELKHLQCLEEELKPLEEVLNLAQSKNFHLRPSYIVSYTNVFPLVLKGSETTFMCEYADETATIVEFLNRWITFSQSIISTLT

SEQ ID NO: 163 - protein, 133 aa, synthetic construct
  source 1..133: note = 2021EP16-H09
APASSSTKKTQLQLEHLLLDLQMILNGINNYKNPKLTRMLTFKFYMPKKATELKHLQCLEEELKPLEEVLNLAQSKNFHLRIRDLLNKINELHRYIKGSETTFMCEYADETATIVEFLNRWITFSQSIISTLT

SEQ ID NO: 164 - protein, 133 aa, synthetic construct
  source 1..133: note = 2021EP16-C10
APASSSTKKTQLQLEHLLLDLQMILNGINNYKNPKLTRMLTFKFYMPKKATELKHLQCLEEELKPLEEVLNLAQSKNFHLRSREVVSRIDAIILELKGSETTFMCEYADETATIVEFLNRWITFSQSIISTLT

SEQ ID NO: 165 - protein, 133 aa, synthetic construct
  source 1..133: note = 2021EP16-G10
APASSSTKKTQLQLEHLLLDLQMILNGINNYKNPKLTRMLTFKFYMPKKATELKHLQCLEEELKPLEEVLNLAQSKNFHLTHRDVISDVNVLALELKGSETTFMCEYADETATIVEFLNRWITFSQSIISTLT

SEQ ID NO: 166 - protein, 133 aa, synthetic construct
  source 1..133: note = 2021EP16-H10
APASSSTKKTQLQLEHLLLDLQMILNGINNYKNPKLTRMLTFKFYMPKKATELKHLQCLEEELKPLEEVLNLAQSKNFHLSAIDLMSSVKVVVWKLKGSETTFMCEYADETATIVEFLNRWITFSQSIISTLT

SEQ ID NO: 167 - protein, 133 aa, synthetic construct
  source 1..133: note = 2021EP16-F05
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 168 - protein, 133 aa, synthetic construct
  source 1..133: note = 2021EP16-C03
APASSSTKKTQLQLEHLLLDLQMILNGINNYKNPKLTRMLTFKFYMPKKATELKHLQCLEEELKPLEEVLNLAQSKNFHLSPPDLIANMAVYFVELKGSETTFMCEYADETATIVEFLNRWITFSQSIISTLT

SEQ ID NO: 169 - protein, 133 aa, synthetic construct
  source 1..133: note = 2021EP16-E07
APASSSTKKTQLQLEHLLLDLQMILNGINNYKNPKLTRMLTFKFYMPKKATELKHLQCLEEELKPLEEVLNLAQSKNFHLRPRDSRRLISVIVFRLKGSETTFMCEYADETATIVEFLNRWITFSQSIISTLT

SEQ ID NO: 170 - protein, 133 aa, synthetic construct
  source 1..133: note = EP596
APASSSTKKTQLQLEHLLLDLQMILNGINNYKNPLLTDMLTRKFYMPKKATELKHLQCLEEELKPLEEVLNLAQSKNFHLTARDAIDNINVIVQELKGSETTFMCEYADETATIVEFLNRWITFSQSIISTLT

SEQ ID NO: 171 - protein, 133 aa, synthetic construct; identical to SEQ ID NO: 167
  source 1..133: note = IL-2 T3A AND C125S

SEQ ID NO: 172 - protein, 451 aa, synthetic construct; identical to SEQ ID NO: 104
  source 1..451: note = EP876

SEQ ID NO: 173 - protein, 603 aa, synthetic construct
  source 1..603: note = EP927
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISDINNIVLELKGSETTFMCEYADETATIVEFLNRWITFSQSIISTLT

SEQ ID NO: 174 - protein, 603 aa, synthetic construct
  source 1..603: note = EP928
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRGLISDINNIVLELKGSETTFMCEYADETATIVEFLNRWITFSQSIISTLT

SEQ ID NO: 175 - protein, 603 aa, synthetic construct
  source 1..603: note = EP929
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISRIDAIVLELKGSETTFMCEYADETATIVEFLNRWITFSQSIISTLT

SEQ ID NO: 176 - protein, 603 aa, synthetic construct
  source 1..603: note = EP934
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISEINNIVLELKGSETTFMCEYADETATIVEFLNRWITFSQSIISTLT

SEQ ID NO: 177 - protein, 603 aa, synthetic construct
  source 1..603: note = EP935
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISRINNIVLELKGSETTFMCEYADETATIVEFLNRWITFSQSIISTLT

SEQ ID NO: 178 - protein, 218 aa, synthetic construct; identical to SEQ ID NO: 117
  source 1..218: note = EP740

SEQ ID NO: 179 - protein, 450 aa, synthetic construct; identical to SEQ ID NO: 116
  source 1..450: note = EP877

SEQ ID NO: 180 - protein, 602 aa, synthetic construct; identical to SEQ ID NO: 121
  source 1..602: note = EP878

SEQ ID NO: 181 - protein, 602 aa, synthetic construct; identical to SEQ ID NO: 122
  source 1..602: note = EP879

SEQ ID NO: 182 - protein, 602 aa, synthetic construct
  source 1..602: note = EP921
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISNISVIVLELKGSETTFMCEYADETATIVEFLNRWITFSQSIISTLT

SEQ ID NO: 183 - protein, 602 aa, synthetic construct
  source 1..602: note = EP922
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRGLISNISVIVLELKGSETTFMCEYADETATIVEFLNRWITFSQSIISTLT

SEQ ID NO: 184 - protein, 602 aa, synthetic construct
  source 1..602: note = EP923
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLT

SEQ ID NO: 185 - protein, 602 aa, synthetic construct
  source 1..602: note = EP924
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISNINNIVLELKGSETTFMCEYADETATIVEFLNRWITFSQSIISTLT

SEQ ID NO: 186 - protein, 602 aa, synthetic construct
  source 1..602: note = EP925
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPKGLISDINNIVLELKGSETTFMCEYADETATIVEFLNRWITFSQSIISTLT

SEQ ID NO: 187 - protein, 602 aa, synthetic construct
  source 1..602: note = EP926
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRGLISDINNIVLELKGSETTFMCEYADETATIVEFLNRWITFSQSIISTLT

SEQ ID NO: 188 - protein, 602 aa, synthetic construct
  source 1..602: note = EP930
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISRIDAIVLELKGSETTFMCEYADETATIVEFLNRWITFSQSIISTLT

SEQ ID NO: 189 - protein, 602 aa, synthetic construct
  source 1..602: note = EP933
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGGGGGSGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRNRGLIGNINNIVLELKGSETTFMCEYADETATIVEFLNRWITFSQSIISTLT

SEQ ID NO: 190 - protein, 360 aa, synthetic construct
  source 1..360: note = EP970
APASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 191 - protein, 360 aa, synthetic construct
  source 1..360: note = EP971
APASSSTKKTQLQLEHLLLDLQMILNGINNYKNPLLTDMLTRKFYMPKKATELKHLQCLEEELKPLEEVLNLAQSKNFHLRPRDLISRIDAIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 192 - protein, 360 aa, synthetic construct
  source 1..360: note = EP972
APASSSTKKTQLQLEHLLLDLQMILNGINNYKNPLLTDMLTRKFYMPKKATELKHLQCLEEELKPLEEVLNLAQSKNFHLRNRGLIGNINNIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 193 - protein, 360 aa, synthetic construct
  source 1..360: note = EP973
APASSSTKKTQLQLEHLLLDLQMILNGINNYKNPLLTDMLTRKFYMPKKATELKHLQCLEEELKPLEEVLNLAQSKNFHLRPKGLISEINNIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 194 - protein, 16 aa, synthetic construct
  source 1..16: note = IL2Rbeta BL2 of EP921
RPKGLISNISVIVLEL

SEQ ID NO: 195 - protein, 16 aa, synthetic construct
  source 1..16: note = IL2Rbeta BL2 of EP922
RPRGLISNISVIVLEL

SEQ ID NO: 196 - protein, 16 aa, synthetic construct
  source 1..16: note = IL2Rbeta BL2 of EP923 and EP970
RPRDLISNISNIVLEL

SEQ ID NO: 197 - protein, 16 aa, synthetic construct
  source 1..16: note = IL2Rbeta BL2 of EP924
RPKGLISNINNIVLEL

SEQ ID NO: 198 - protein, 16 aa, synthetic construct
  source 1..16: note = IL2Rbeta BL2 of EP925 and EP927
RPKGLISDINNIVLEL

SEQ ID NO: 199 - protein, 16 aa, synthetic construct
  source 1..16: note = IL2Rbeta BL2 of EP929, EP930, and EP971
RPRDLISRIDAIVLEL

SEQ ID NO: 200 - protein, 16 aa, synthetic construct
  source 1..16: note = IL2Rbeta BL2 of EP933 and EP972
RNRGLIGNINNIVLEL

SEQ ID NO: 201 - protein, 16 aa, synthetic construct
  source 1..16: note = IL2Rbeta BL2 of EP934 and EP973
RPKGLISEINNIVLEL

SEQ ID NO: 202 - protein, 16 aa, synthetic construct
  source 1..16: note = IL2Rbeta BL2 of EP935
RPKGLISRINNIVLEL

SEQ ID NO: 203 - protein, 133 aa, synthetic construct
  source 1..133: note = EP921
APASSSTKKTQLQLEHLLLDLQMILNGINNYKNPLLTDMLTRKFYMPKKATELKHLQCLEEELKPLEEVLNLAQSKNFHLRPKGLISNISVIVLELKGSETTFMCEYADETATIVEFLNRWITFSQSIISTLT

SEQ ID NO: 204 - protein, 133 aa, synthetic construct
  source 1..133: note = EP922
APASSSTKKTQLQLEHLLLDLQMILNGINNYKNPLLTDMLTRKFYMPKKATELKHLQCLEEELKPLEEVLNLAQSKNFHLRPRGLISNISVIVLELKGSETTFMCEYADETATIVEFLNRWITFSQSIISTLT

SEQ ID NO: 205 - protein, 133 aa, synthetic construct
  source 1..133: note = EP923 and EP970
APASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLT

SEQ ID NO: 206 - protein, 133 aa, synthetic construct
  source 1..133: note = EP924
APASSSTKKTQLQLEHLLLDLQMILNGINNYKNPLLTDMLTRKFYMPKKATELKHLQCLEEELKPLEEVLNLAQSKNFHLRPKGLISNINNIVLELKGSETTFMCEYADETATIVEFLNRWITFSQSIISTLT

SEQ ID NO: 207 - protein, 133 aa, synthetic construct
  source 1..133: note = EP925 and EP927
APASSSTKKTQLQLEHLLLDLQMILNGINNYKNPLLTDMLTRKFYMPKKATELKHLQCLEEELKPLEEVLNLAQSKNFHLRPKGLISDINNIVLELKGSETTFMCEYADETATIVEFLNRWITFSQSIISTLT

SEQ ID NO: 208 - protein, 133 aa, synthetic construct
  source 1..133: note = EP929, EP930, and EP971
APASSSTKKTQLQLEHLLLDLQMILNGINNYKNPLLTDMLTRKFYMPKKATELKHLQCLEEELKPLEEVLNLAQSKNFHLRPRDLISRIDAIVLELKGSETTFMCEYADETATIVEFLNRWITFSQSIISTLT

SEQ ID NO: 209 - protein, 133 aa, synthetic construct
  source 1..133: note = EP933 and EP972
APASSSTKKTQLQLEHLLLDLQMILNGINNYKNPLLTDMLTRKFYMPKKATELKHLQCLEEELKPLEEVLNLAQSKNFHLRNRGLIGNINNIVLELKGSETTFMCEYADETATIVEFLNRWITFSQSIISTLT

SEQ ID NO: 210 - protein, 133 aa, synthetic construct
  source 1..133: note = EP934 and EP973
APASSSTKKTQLQLEHLLLDLQMILNGINNYKNPLLTDMLTRKFYMPKKATELKHLQCLEEELKPLEEVLNLAQSKNFHLRPKGLISEINNIVLELKGSETTFMCEYADETATIVEFLNRWITFSQSIISTLT

SEQ ID NO: 211 - protein, 133 aa, synthetic construct
  source 1..133: note = EP935
APASSSTKKTQLQLEHLLLDLQMILNGINNYKNPLLTDMLTRKFYMPKKATELKHLQCLEEELKPLEEVLNLAQSKNFHLRPKGLISRINNIVLELKGSETTFMCEYADETATIVEFLNRWITFSQSIISTLT

SEQ ID NO: 212 - protein, 20 aa, synthetic construct
  source 1..20: note = Signal Peptide
METDTLLLWVLLLWVPGSTG

(19) United States
(12) Patent Application Publication
(10) Pub. No.: US 2025/0326813 A1
(43) Pub. Date: Oct. 23, 2025

(54) ENGINEERED INTERLEUKIN-2 RECEPTOR BETA REDUCED-BINDING AGONIST

(71) Applicant: Elpis Biopharmaceuticals, Lexington, MA (US)

(72) Inventors: Kehao ZHAO, Lexington, MA (US); Yan CHEN, Lexington, MA (US); Samuel Clement HASSAN, Lexington, MA (US); Ning JIANG, Lexington, MA (US); Katie O'CALLAGHAN, Lexington, MA (US)

(21) Appl. No.: 18/864,369

(22) PCT Filed: May 9, 2023
(86) PCT No.: PCT/US2023/066784

§ 371 Date: Nov. 8, 2024

Related U.S. Application Data
(60) Provisional application No. 63/340,294, filed on May 10, 2022.
(60) Provisional application No. 63/391,243, filed on Jul. 21, 2022.

(51) Int. Cl. C07K 14/55; A61K 35/17; A61K 38/00; A61K 40/11; A61K 40/31; A61K 40/42; A61K 45/06; A61P 35/00; C07K 16/28; C12N 5/0783

(52) U.S. Cl. CPC C07K 14/55; A61K 35/17; A61K 40/11; A61K 40/31; A61K 40/4214; A61K 45/06; A61P 35/00; C07K 16/2827; C12N 5/0636; A61K 38/00; A61K 2239/15; C07K 2319/30; C07K 2319/31; C12N 2510/00

(57) ABSTRACT

Provided herein are engineered IL2 polypeptides IL2Rβ reduced-binding agonists and fusion proteins thereof. Also provided are methods of modulating an immune response by administering an engineered IL2 polypeptide reduced-binding agonist or a fusion protein thereof.

### REFERENCE TO AN ELECTRONIC SEQUENCE LISTING

The contents of the electronic sequence listing (300096_403WO_SEQUENCE_LISTING.xml; Size: 266,559 bytes; and Date of Creation: May 8, 2023) is herein incorporated by reference in its entirety.

### BACKGROUND

Interleukin-2 (IL2) is a cytokine that modulates lymphocyte proliferation and activation. It has a length of 133 amino acids and the structure includes four antiparallel, amphipathic C-helices. IL2 mediates its action by binding to IL2 receptors (IL2R), which includes up to three individual subunits. Association of all three subunits, the interleukin-2 receptor alpha chain (IL2Rα, or CD25), interleukin-2 receptor beta chain (IL2Rβ, or CD122), and interleukin-2 receptor gamma chain (IL2Rγ, or CD132), results in a trimeric IL2Rαβγ, which is a high-affinity receptor for IL2. Association of the IL2Rβ and IL2Rγ subunits results in the dimeric receptor IL2Rβγ, and is termed an intermediate affinity IL2R. The IL2Rα subunit forms a monomeric low affinity IL2 receptor. Expression of IL2Rα is involved in the expansion of immunosuppressive regulatory T cells (Tregs); whereas dimeric IL2Rβγ can result in cytolytic CD8⁺ T cell and NK cell proliferation and killing in the absence of IL2Rα.

### BRIEF SUMMARY

The present disclosure provides a rationally designed engineered IL2 polypeptides having amino acid substitutions in IL2Rβ binding region 2 that reduce binding to IL2Rβ compared to wild-type IL2.

In one aspect, the present disclosure provides an engineered interleukin-2 (IL2) polypeptide comprising an engineered IL2 receptor β (IL2Rβ) binding region 2 motif comprising:

(SEQ ID NO: 3)
X₁-X₂-X₃-X₄-X-₅-X₆-X₇-X₈-X₉-X₁₀-X₁₁-X₁₂-X₁₃-
X₁₄-X₁₅-X₁₆,

wherein:
X₁ comprises a residue selected from R, S, L, N, F, K, or T;
X₂ comprises a residue selected from A, F, S, L, R, T, I, H, P, or N;
X₃ comprises a residue selected from K, R, T, S, I, or P;
X₄ comprises a residue selected from G, D, R, A, Q, H, N, Y, or E;
X₅ comprises a residue selected from I, P, T, S, K, F, V, or L;
X₆ comprises a residue selected from I, R, V, M, T, or L;
X₇ comprises a residue selected from A, R, M, I, S, N, G, or S;
X₈ comprises a residue selected from E, N, H, T, K, Y, S, L, V, D, or R;
X₉ comprises a residue selected from I, V, A, T, L, T, or M;
X₁₀ comprises a residue selected from N, G, V, Y, I, W, R, K, Q, A, D, S, or D;
X₁₁ comprises a residue selected from F, G, V, N, T, I, R, E, or A;
X₁₂ comprises a residue selected from I, S, R, V, P, G, T, L, M, F, or Y;
X₁₃ comprises a residue selected from V, I, F, D, P, H, A, V, or L;
X₁₄ comprises a residue selected from L, Q, R, E, P, K, H, W, F, or V;
X₁₅ comprises a residue selected from A, E, L, K, V, D, Y, R, or Q; and
X₁₆ comprises a residue selected from L or I, and
wherein the engineered IL2Rβ binding region 2 motif does not comprise SEQ ID NO:2 and the engineered IL2 polypeptide binds to IL2Rβ at a reduced affinity compared to a wild-type IL2.

In some aspects, the present disclosure provides an engineered IL2 polypeptide, comprising a sequence have at least 90% sequence identity to a sequence selected from a group consisting of: SEQ ID NOS:46-102 and 147-169, and 203-211. In some aspects, the present disclosure provides an engineered IL2 polypeptide, comprising a sequence selected from a group comprising or consisting of: SEQ ID NOS:46-102, and 147-169, and 203-211.

In some aspects, the present disclosure provides a fusion polypeptide comprising a first polypeptide sequence and a second polypeptide sequence, wherein the first polypeptide sequence comprises an engineered IL2 polypeptide as provided herein. In some aspects, the second polypeptide sequence of the fusion protein include a Fc domain, antibody, antigen binding moiety, cytokine, half-life extending molecule, tag or marker polypeptide, targeting domain, transport molecule, immunotoxin, NKG2D, linker sequence, PEGylation, chemically linked small molecule, nucleic acid, or any combination thereof. In some aspects, the second polypeptide sequence comprises an antibody heavy chain constant region. In some aspects, the antibody heavy chain constant region is human IgG heavy chain constant region. In some aspects, the second polypeptide comprises an antigen binding moiety. For example, the antigen binding moiety is capable of binding PD-L1, PD-1, CTLA-4, TIM3, LAG3, B7-H2, B7-H3, CD4, CD8, or a cellular marker.

In some aspects, the present disclosure provides a monovalent engineered IL2-Fc fusion polypeptide complex, comprising: (a) a first polypeptide comprising a fusion polypeptide as described herein, and (b) a second polypeptide that forms a dimer with the first protein. In some aspects, the second polypeptide comprises a heavy chain constant region.

In some aspects, the present disclosure provides a protein complex, comprising a first polypeptide that is a fusion polypeptide as described herein and a second polypeptide comprising an antigen binding moiety. In some aspects, the antigen binding moiety is capable of binding PD-L1, PD-1, CTLA-4, TIM3, LAG3, B7-H2, B7-H3, CD4, CD8, or a cellular marker.

In some aspects, the present disclosure provides a bifunctional fusion protein, comprising: (a) an engineered IL2 polypeptide comprising a sequence of any one of claims 1-2524; and (b) an antigen-binding moiety. In some aspects, the antigen binding moiety is capable of binding PD-L1, PD-1, CTLA-4, TIM3, LAG3, B7-H2, B7-H3, CD4, CD8, or a cellular marker.

In some aspects, the present disclosure provides an isolated polynucleotide encoding at least one polypeptide disclosed herein. In some aspects, the present disclosure provides an expression vector comprising the polynucleotide encoding at least one polypeptide disclosed herein. In some aspects, the present disclosure provides a modified cell comprising the isolated polynucleotide or the expression vector disclosed herein.

In some aspects, the present disclosure provides a pharmaceutical composition comprising an engineered IL2 polypeptide, a fusion polypeptide, a protein complex, a bifunctional fusion protein, a polynucleotide, a vector, or a modified cell as disclosed herein, and a pharmaceutically acceptable carrier.

In some aspects, the present disclosure provides a method of modulating an immune response in a subject in need thereof, comprising administering an effective amount of an engineered IL2 polypeptide, a fusion polypeptide, a protein complex, a bifunctional fusion protein, a polynucleotide, a vector, a modified cell, or pharmaceutical compositions as disclosed herein.

In some aspects, the present disclosure provides a of treating a disease in a subject in need thereof, comprising administering an effective amount of an engineered IL2 polypeptide, a fusion polypeptide, a protein complex, a bifunctional fusion protein, a polynucleotide, a vector, a modified cell, or pharmaceutical compositions as disclosed herein to the subject.

In some aspects, the present disclosure provides a cell culture medium comprising an engineered IL2 polypeptide, fusion polypeptide, protein complex, bifunctional fusion protein, polynucleotide, vector, or cell disclosed herein. In some aspects, disclosed herein is a method of culturing a cell, comprising incubating a cell with the culture medium comprising an engineered IL2 polypeptide, fusion polypeptide, protein complex, bifunctional fusion protein, polynucleotide, vector, or cell disclosed herein.

In some aspects, the present disclosure provides a transgenic immune cell comprising an engineered IL2 polypeptide, a fusion polypeptide, a protein complex, a bifunctional fusion protein, a polynucleotide, or a vector as described herein. In some aspects, the immune cell is a CD4+ T cell, a CD8+ T cell, a γδ T cell, a NK cell, a T regulatory cell, or any combination thereof. In some aspects, the immune cell further comprises a chimeric antigen receptor (CAR). In some aspects, the engineered IL2 polypeptide, fusion polypeptide, protein complex, or bifunctional fusion protein is secreted by the transgenic immune cell or expressed/localized to the surface of the cell.

### BRIEF DESCRIPTION OF THE SEVERAL VIEWS OF THE DRAWINGS

FIG. 1. shows an SDS-PAGE of individual engineered IL2 after Ni column purification.

FIGS. 2A-2B show the identification of IL2Rβ reduced binding mutations by ELISA. FIG. 2A shows binding to IL2Rα. FIG. 2B shows binding to IL2Rβ.

FIGS. 3A-3D show the percent of cells that were p-STAT5 positive when treated with titrations of engineered IL2 compared to wild-type IL2. FIG. 3A shows results for CD4+ T cells. FIG. 3B shows results for CD8+ T cells. FIG. 3C shows results for NK cells. FIG. 3D shows results for T reg cells.

FIG. 4 shows an SDS-PAGE of individual engineered IL2 after Ni column purification.

FIGS. 5A-5B show binding of mammalian produced IL2 EP575 back-mutation muteins to IL2 receptors in ELISA. FIG. 5A shows binding to IL2Rα. FIG. 5A shows binding to IL2Rβ.

FIGS. 6A-6D show pSTAT5 activities of back-mutations of EP575 to human immune cells. FIG. 6A shows results for CD4+ T cells. FIG. 6B shows results for CD8+ T cells. FIG. 6C shows results for NK cells. FIG. 6D shows results for T reg cells.

FIGS. 7A-7D show pSTAT5 activities of back-mutations of EP658 to human immune cells. FIG. 7A shows results for CD4+ T cells. FIG. 7B shows results for CD8+ T cells. FIG. 7C shows results for NK cells. FIG. 7D shows results for T reg cells.

FIGS. 8A-8D show pSTAT5 activities of back-mutations of EP661 to human immune cells. FIG. 8A shows results for CD4+ T cells. FIG. 8B shows results for CD8+ T cells. FIG. 8C shows results for NK cells. FIG. 8D shows results for T reg cells.

FIG. 9 shows binding of engineered IL2 to cell surface IL2 receptors.

FIGS. 10A-10C show diagrams of example IL2-Fc fusion and bifunctional proteins. FIG. 10A shows an example of a monovalent IL2-Fc. FIG. 10B shows an example of an IL2-Fc in a protein complex with an antibody Fab-Fc. FIG. 10C shows an example of an engineered IL2-antibody-antibody fusion protein complexed with an antibody Fab-Fc (e.g., immunoconjugate).

FIGS. 11A-11C show ELISA binding of engineered IL2 fused to anti-PD-L1 antibody Fab-Fc. FIG. 11A shows binding to PD-L1. FIG. 11B shows binding to IL2α. FIG. 11C shows binding to IL2β.

FIGS. 12A-12D show p-STAT5 activity in cells when treated with titrations of engineered IL2 fused to anti-PD-L1 antibody Fab-Fc. FIG. 12A shows results for CD4+ T cells. FIG. 12B shows results for CD8+ T cells. FIG. 12C shows results for NK cells. FIG. 12D shows results for T reg cells.

FIGS. 13A-13B show results of a safety assessment in mice using an engineered IL2 (EP567) fused to an anti-PD-L1 antibody. FIG. 13A shows relative body weight. FIG. 13B shows lung weight.

FIGS. 14A-14D show immune modulation activities in mice treated with a bifunctional protein comprising EP567 fused to anti-PD-L1 antibody. FIG. 14A shows percent of live CD4+ T cells. FIG. 14B shows percent of live CD8+ T cells. FIG. 14C shows percent of live NK cells. FIG. 14D shows percent of live T reg cells.

FIGS. 15A-15C show immune modulation activities in mice treated with a bifunctional protein comprising EP567 fused to anti-PD-L1 antibody. FIG. 15A shows percent Ki67+ CD4+ T cells. FIG. 15B shows percent of Ki67+ CD8+ T cells. FIG. 15C shows percent of Ki67+ T reg cells.

FIGS. 16A-16C show immune modulation activities in mice treated with a bifunctional protein comprising EP567 fused to anti-PD-L1 antibody. FIG. 16A shows percent CD69+ CD4+ T cells. FIG. 16B shows percent of CD69+ CD8+ T cells. FIG. 16C shows percent of CD69+ T reg cells.

FIGS. 17A-17B show results of a safety assessment in mice using engineered IL2 polypeptides fused to an anti-PD-L1 antibody. FIG. 17A shows percent body weight. FIG. 17B shows lung weight.

FIGS. 18A-18D show immune activities in mice treated with engineered IL2 polypeptides fused to an anti-PD-L1 antibody. FIG. 18A shows percent of CD8+ T cells. FIG. 18B shows percent of live CD4+ T cells. FIG. 18C shows percent of NK cells. FIG. 18D shows percent of live T reg cells.

FIGS. 19A-19D show immune modulation activities in mice treated engineered IL2 polypeptides fused to an anti-PD-L1 antibody. FIG. 19A shows percent Ki67+ CD4+ T cells. FIG. 19B shows percent of Ki67+ CD8+ T cells. FIG. 19C shows percent of Ki67+ T reg cells. FIG. 19D shows percent of Ki67+ NK cells.

FIGS. 20A-20B show subtype CD4 and CD8 T cell immune modulation activities in mice treated engineered IL2 polypeptides fused to an anti-PD-L1 antibody. FIG. 20A shows the percent of subtype CD4+ T cells. FIG. 20B shows the percent of subtype CD8+ T cells.

FIG. 21 shows pharmacokinetics of an EP661/anti-PD-L1 Bispecific in a mouse model.

FIGS. 22A-22C show anti-tumor activities of IL2 fused to anti-PD-L1 bispecific in a B16F10-hPD-L1 mouse model. FIG. 22A shows average tumor volume. FIG. 22B shows relative body weight change. FIG. 22C shows tumor volume of individual mouse treated with atezolizumab, EP658/hPD-L1, EP661/hPD-L1, or EP669/hPD-L1.

FIGS. 23A-23D show peripheral immune cell activation in vivo. FIG. 23A shows activation of CD8+ T cells. FIG. 23B shows activation of NK cells. FIG. 23C shows activation of CD4+ T cells. FIG. 23C shows activation of T reg cells.

FIGS. 24A-24B show peripheral immune cell activation in vivo. FIG. 24A shows raw counts of CD8+ T cells to T reg cells. FIG. 24B shows raw counts of NK cells to T reg cells.

FIGS. 25A-25B show anti-tumor activities of engineered IL2 fused to anti-PD-L1 bispecific in a B16F10-hPD-L1 mouse model. FIG. 25A tumor volume over time in mice treated with vehicle control, EP669/PDL1 (3 mg/kg), or EP669/PDL1 (10 mg/kg). FIG. 25B shows body weight over time in mice treated with vehicle control, EP669/PDL1 (3 mg/kg), or EP669/PDL1 (10 mg/kg).

FIGS. 26A-26B show anti-tumor activities of engineered IL2 fused to anti-PD-1 bispecific in a B16F10-hPD-L1 mouse model compared to vehicle control and PD-1-targeted IL-2v antibody fusion protein RO7284755. FIG. 26A tumor volume over time in mice treated with vehicle control, EP661/PD1 (3 mg/kg), or RO7284755 (1 mg/kg). FIG. 26B shows body weight over time in mice treated with vehicle control, EP661/PD1 (3 mg/kg), or RO7284755 (1 mg/kg).

FIG. 27 shows the anti-tumor activities of engineered IL2 fused to mouse anti-PD-1 bispecific (EP661/mPD-1) compared to vehicle control and anti-mPD-1 treatment (10 mg/kg) in a MC38 mouse model.

FIGS. 28A-28D show peripheral immune cell activation in vivo in mice treated with EP661/mPD-1 (1 mg/kg) compared to vehicle control and anti-mPD-1 treatment (10 mg/kg). FIG. 28A shows the number of cells/mL of FoxP3− CD4+ T cells. FIG. 28B shows the number of cells/mL of CD8+ T cells. FIG. 28C shows the number of cells/mL of NK cells. FIG. 28D shows the number of cells/mL of FoxP3+ CD25+ regulatory T cells.

FIGS. 29A-29D show p-STAT5 activity in cells when treated with titrations of engineered IL2 fused to anti-PD-L1 antibody Fab-Fc or anti-PD-1 antibody Fab-Fc compared to WT IL2 fused to anti-PD-L1 antibody, an anti-PD-L1 antibody, and PD-1-targeted IL-2v antibody fusion protein RO7284755. FIG. 29A shows p-STAT5 activity in CD4+ FoxP3− T cells. FIG. 29B shows p-STAT5 activity in CD8+ T cells. FIG. 29C shows p-STAT5 activity in NK cells. FIG. 29D shows p-STAT5 activity in regulatory T cells.

FIGS. 30A-30B show immunohistochemistry staining of infiltrating CD8+ cells. FIG. 30A shows tumor infiltrating cells a mouse treated with vehicle control. FIG. 30B shows tumor infiltrating cells a mouse treated with EP669/PDL1 (3 mg/kg, Q4D).

FIGS. 31A-31C show fold-change in total white blood cells (WBC), lymphocytes and neutrophils from pre-treatment (Day 0) to Day 2. FIG. 31A shows WBC fold-change. FIG. 31B shows lymphocyte fold-change. FIG. 31C shows neutrophil fold-change.

FIGS. 32A-32B show blood phenotypes. FIG. 32A shows percentage of CD4+ FoxP3− T cells. FIG. 32B shows the percentage of CD8+ T cells.

FIGS. 33A-33B show the anti-tumor activities of engineered IL2 fused to anti-PD-1 bispecific (EP661/PD-1, EP930/PD-1, EP933/PD-1, EP935/PD-1) compared to vehicle control and RO7284775 treatment in a MC38 mouse model. FIG. 33A shows tumor volume over time. FIG. 33B shows body weight over time.

FIGS. 34A-34B show blood phenotypes. FIG. 34A shows percentage of CD4+ FoxP3− T cells. FIG. 34B shows the percentage of CD8+ T cells.

### DETAILED DESCRIPTION

Presented herein are rationally designed engineered IL2 polypeptides having amino acid substitutions in IL2Rβ binding region 2 that reduce binding to IL2Rβ compared to wild-type IL2. The engineered IL2 polypeptides are IL2Rβ reduced-binding agonists that provide the advantage of stimulating NK cells and T effector cells while providing improved safety and durable immune modulation compared to wild-type IL2. Thus, the engineered IL2Rβ agonists are useful for modulating or activating an immune response, for example, for treatment of cancer.

### Definitions

In the present description, any concentration range, percentage range, ratio range, or integer range is to be understood to include any values or subranges within the recited range unless otherwise indicated. As used herein, the term “about” means ±20% of the indicated range or value unless otherwise indicated.

It should also be noted that the term “or” is generally employed in its sense including “and/or” (i.e., to mean either one, both, or any combination thereof of the alternatives) unless the content dictates otherwise.

Also, as used in this specification and the appended claims, the singular forms “a,” “an,” and “the” include plural referents unless the content dictates otherwise.

The terms “include,” “have,” “comprise” and their variants are used synonymously and to be construed as non-limiting.

The term “a combination thereof” as used herein refers to all possible combinations of the listed items preceding the term. For example, “A, B, C, or a combination thereof” is intended to refer to any one of: A, B, C, AB, AC, BC, or ABC. Similarly, the term “combinations thereof” as used herein refers to all possible combinations of the listed items preceding the term. For instance, “A, B, C, and combinations thereof” is intended to refer to all of: A, B, C, AB, AC, BC, and ABC.

The term “interleukin-2 or “IL2” as used herein, refers to an IL2 from any vertebrate source, including mammals such humans or mice, unless otherwise indicated. The term encompasses precursor or unprocessed IL2, as well as any form of IL2 that results from cellular processing. The term also encompasses naturally occurring variants of IL2, such as splice variants or allelic variants. “Wild-type” or “native” when used in reference to IL2 is intended to mean the mature IL2 molecule (e.g., SEQ ID NO: 1). The term “engineered IL2” or “engineered IL2 polypeptide” as used herein encompasses an IL2 having at least one residue that differs from a native or wild-type IL2, and includes full-length IL2, truncated forms of IL2, and forms where IL2 is linked or fused with another molecule, such as another polypeptide. The various forms of engineered IL2 are characterized in having at least one amino acid substitution affecting the interaction of IL2 with IL2Rβ. The engineered IL2 referred to herein may be IL2Rβ reduced-binding agonists. IL2Rβ reduced-binding agonists have reduced binding to IL2Rβ compared to wild-type IL2 or, e.g., an IL2 having a T3A and C125S substitution relative to SEQ ID NO:1, e.g., SEQ ID NO:171.

IL2Rβ binding region 1 and IL2Rβ binding region 2 are responsible for IL2 binding to IL2Rβ. “IL2Rβ binding region 1” as used herein refers to residues 11-23 of wild-type or native human IL2. “IL2Rβ binding region 2” as used herein refers to residues 81-96 of wild-type or native human IL2. The amino acid sequence of IL2Rβ binding region 2 is provided in SEQ ID NO: 2.

IL2Rα binding region 1 and IL2Rα binding region 2 are responsible for IL2 binding to IL2Rα. “IL2Rα binding region 1” as used herein refers to residues 34-45 of wild-type or native human IL2.

As used herein, the term “engineered,” “recombinant,” or “non-natural” refers to a polypeptide/protein, nucleic acid molecule, vector, organism, microorganism, or cell that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

The term “substitution” or “residue substitution” as used herein refers to replacement of a native or wild-type residue with a different residue. Similarly, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). Various identifiers may be used herein to indicate the same residue substitution. For example, a substitution from threonine at position 3 to alanine can be indicated as T3A or 3A.

As used herein, “nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Fusion polypeptide” or “fusion protein” refers to a polypeptide that is encoded by at least two different DNA sequences corresponding to genes or fragments thereof, which are not naturally expressed from the same gene. An example of a fusion polypeptide is an engineered IL2-Fc fusion polypeptide, which includes an amino acid sequence of an engineered IL2 polypeptide and an amino acid sequence of an Fc domain.

A “protein complex” or “multiprotein complex” refers to a group of two or more associated polypeptide chains that interact to form a quaternary structure. The complex may be formed under energetically favorable circumstances. For example, a protein complex may form due to ionic interactions and/or hydrophobic interactions. A protein complex may comprise two or more protein subunits that are linked by one or more disulfide bonds or disulfide linkages. An antibody comprising at least one heavy chain and at least one light chain is an example of a protein complex. A protein complex can include one or more fusion proteins. For example, an IL2-Fc fusion polypeptide may form a protein complex with a heavy chain and light chain of an antibody.

A “bifunctional fusion protein” or “bispecific” refers to a protein, fusion protein, and/or heterodimeric protein pair that includes one or more functional domains. Examples of functional domains include an antigen-binding site, antibody fragments (e.g., Fab, scFv, etc.), an antibody heavy chain and light chain, and cytokines (e.g., IL-2, IL-15). Bifunctional fusion protein or bispecific can refer an antibody that comprises a fusion to a non-antibody polypeptide, such as a cytokine. For example, a bifunctional protein can include an antibody heavy chain and light chain wherein the heavy chain constant region is fused to engineered IL-2. In addition, a bifunctional fusion protein can comprise an antibody heavy chain and light chain wherein the heavy chain constant region can form a heterodimer with polypeptide or protein that does not comprise an antigen binding site. For example, a bifunctional fusion protein can comprise a heavy chain, a light chain, and an engineered IL-2 fusion protein that comprises an antibody Fc domain capable of forming a heterodimer with the Fc domain of the antibody heavy chain.

As used herein, “percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and a ligand). The affinity of a molecule-X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by methods known by persons of skill in the art, including those described herein.

“Immunoglobulin” refers to a protein having the structure of a naturally occurring antibody. As an example, immunoglobulins of the IgG class are heterotetrameric glycoproteins with two light chains and two heavy chains that are disulfide-bonded. From N- to C-terminus, the heavy chains each have a variable region (VH), also called a variable heavy domain or a heavy chain variable domain, followed by three constant domains (CH1, CH2, and CH3), also called a heavy chain constant region. Similarly, from N- to C-terminus, light chain each have a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ1 (IgG1), γ2 (lgG2), γ3 (IgG3), γ4 (lgG4), α1 (IgA1) and α2 (IgA2). The light chain of an immunoglobulin may be assigned to one of two types, called kappa (κ) and lambda (λ), based on the sequence of its constant domain. An immunoglobulin includes two Fab molecules and an Fc domain, linked via the immunoglobulin hinge region.

“Fc domain” or “Fc region” as used herein refers to a polypeptide derived from a C-terminal region of an immunoglobulin heavy chain that contains at least a portion of the constant region. The term includes polypeptides having a native sequence Fc region, or variants thereof. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Examples of Fc regions are disclosed in U.S. Pat. Nos. 7,317,091; 8,735,545; 7,371,826; 7,670,600; and 9,803,023; all of which are incorporated by reference in their entirety.

The term “antibody” as used herein encompasses various antibody structures, including but not limited to monoclonal antibodies, polyclonal antibodies, multispecific antibodies (e.g., bispecific antibodies, bifunctional antibodies), antibody fusion proteins, antibodies that for heterodimers in engineered proteins, and antibody fragments so long as they exhibit the desired antigen-binding activity.

An “antibody fragment” refers to a polypeptide or protein other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

“Antigen binding moiety” or “antigen-binding site” are used interchangeably herein and refer to the site (i.e., amino acid residues) of an antigen binding molecule (e.g., antibody) that provides interaction with the antigen epitope. An antigen binding moiety may include one or more antibody variable domains (also called antibody variable regions). In human antibodies, the antigen binding site is formed by amino acid residues of the N-terminal variable (“V”) regions of the heavy (“H”) and light (“L”) chains. The “hypervariable regions” are three highly divergent stretches within the V regions of the heavy and light chains which are interposed between “framework regions,” (“FR”), which are relatively conserved flanking stretches. The term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen. The three hypervariable regions of each of the heavy (“H”) and light (“L”) chains are referred to as “complementarity-determining regions” or “CDRs.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. An antigen binding site can comprise an antibody light chain variable region (VL) and an antibody heavy chain variable region (VH). Examples of antigen binding moieties include immunoglobulins, Fab molecules, scFv, bispecific antibodies, diabodies, bi-specific T-cell engagers, and nanobodies. Specific examples of antigen binding moieties include nivolumab, pembrolizumab, pidilizumab, atezolizumab, ipilimumab, tremelimumab, rituximab, ocrelizumab, obinutuzumab, ofatumumab, ibritumomab tiuxetan, tositumomab, ublituximab, and bevacizumab.

Numbering of CDR and framework regions may be according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering and Receptor Classification (ANARCI) software tool (2016, Bioinformatics 15:298-300). The CDRs of an antigen-binding site can be determined according to known methods, such as the Kabat, Chothia, EU, IMGT, and AHo as described above. The CDRs determined under these definitions typically include overlapping or subsets of amino acid residues when compared against each other. The heavy chain CDRs and light chain CDRs of an antibody can be defined using different numbering conventions. For example, in certain embodiments, the heavy chain CDRs are defined according to Chothia, supra, and the light CDRs are defined according to Kabat, supra. CDRH1, CDRH2 and CDRH3 denote the heavy chain CDRs, and CDRL1, CDRL2 and CDRL3 denote the light chain CDRs.

“Fab molecule” or “antigen binding fragment” is an antigen binding fragment of an antibody that includes the variable domain and constant domain of a light chain, and a variable domain and a CH1 domain of a heavy chain.

“Single chain variable domain” or “scFv” refers to an antigen binding moiety that includes variable regions of a heavy chain and light chain, which are linked by a linker peptide.

“Bispecific antibody,” refers to an artificial antibody with two different antigen binding sites. Bispecific antibody can refer to a full immunoglobulin protein with two different antigen binding sites, or can refer to other molecules having two antigen binding moieties, such as a fusion protein including two Fabs or two scFvs.

“Diabody” refers to a class of antigen binding molecules that are bivalent and bispecific. The fragments comprise a heavy-chain variable domain (VH) connected to a light-chain variable domain (VL) on the same polypeptide chain (VH-VL). By using a linker that is too short to allow pairing between the two domains on the same chain, the domains are forced to pair with the complementary domains of another chain and create two antigen-binding sites.

“Bi-specific T-cell engager” refers to a class of bispecific antibodies having a first antigen binding moiety that binds to a T cell (e.g., by binding CD3), and a second antigen binding moiety that binds a different antigen (e.g., a tumor antigen).

“VHH antibody,” “Nanobody,” or “single domain antibody” refers to an antigen binding moiety that consists of a single monomeric variable antibody domain.

“Transferrin” is an iron transporter protein that may be used in a fusion protein to extend half-life. Human transferrin has a half-life of 12 days in serum.

“Cytokine” as used herein refers to a class of small (<25 kDa) proteins that are involved in cell signaling and immunomodulation. Cytokines include, for example, IL2, interleukin-10 (IL-10), interleukin-1 (IL-1), interleukin-17 (IL-17), interleukin-18 (IL-18), interferon α, interferon β, interferon γ, TGF-β1, TGF-β2, and TGF-β3, chemokine (C-C motif) ligand 2 (CCL2), and chemokine (C-C motif) ligand 19 (CCL19).

“Half-life extending molecule” as used herein refers to a molecule that when attached (e.g., covalently) to a second molecule, extends the half-life of the second molecule. Examples of half-life extending molecules include an Fc domain, human serum albumin (HSA), an HSA binding molecule, polyethylene glycol (PEG), and polypropylene glycol (PPG).

“Human serum albumin” or “HSA” refers to the serum albumin found in human blood. The commonly used form of HSA has a molecular mass of 66.5 kDa and a half-life of approximately 20 days. Examples of HSA molecules are disclosed in U.S. Pat. Nos. 8,143,026 and 7,189,690, which are incorporated by reference in their entirety.

“HSA binding molecule” refers to a molecule that specifically binds to human serum albumin (HSA), such as an antigen binding moiety having an HSA binding domain.

“Polyethylene glycol” or “PEG,” also referred to as polyethylene oxide or polyoxyethylene is a polyether polymer that may be used to extend half-life.

“Polypropylene glycol” or “PPG,” also referred to as polypropylene oxide, is a polymer of propylene glycol that may be used to extend half-life.

A “subject” according to any of the above embodiments is a mammal. Mammals include but are not limited to, domesticated animals (e.g., cows, sheep, cats, dogs, and horses), primates (e.g., human and non-human primates such as monkeys), rabbits, and rodents (e.g., mice and rats). Preferably the subject is a human.

“Modulating an immune response” may include one or more of a general increase, an increase in T effector cell response (e.g., cytotoxicity against tumor cells and virus infected cells), an increase in B cell activation, restoration of lymphocyte activation and proliferation, an increase in the expression of IL2 receptors, an increase in T cell responsiveness, an increase in natural killer cell activity or lymphokine-activated killer (LAK) cell activity, a decrease in regulatory T cells response to other T cells, and the like.

“Regulatory T cell” or “Treg cell” refers to a specialized type of CD4+ T cell that can function to suppress the responses of other T cells. Treg cells express the a-subunit of the IL2 receptor (CD25) and the transcription factor forkhead box P3 (FOXP3) (Sakaguchi, Annu Rev Immunol 22, 531-62 (2004)), and are involved in the induction and maintenance of peripheral self-tolerance to antigens, including those expressed by tumors. Treg cells require IL2 for their function and development and induction of their suppressive characteristics.

“T effector cells” refers to a population of T cells that respond to stimulus, such as IL2. T effector cells include CD8+ cytotoxic T cells and CD4+ helper T cells. As used herein, T effector cell does not include a T regulatory cell.

“Natural Killer cells” or “NK cells” are a component of the innate immune system and are cytotoxic lymphocytes that play a major role in rejection of tumors and virus infected cells.

“Chimeric antigen receptors” or “CARs” are engineered antigen binding receptors that, when expressed in certain types of immune cells, activate the immune cell upon antigen binding. CARs typically include an extracellular domain comprising an antigen binding moiety (e.g., an scFv), a transmembrane domain, and an intracellular immune signaling domain (e.g., including signaling domains from CD3ζ, 4-1BB, and/or CD28). CARs may be expressed, for example, by T cells or NK cells, and may include an antigen binding moiety that targets a cancer antigen, such as CD19 or ROR1.

“Treatment,” “treating” or “ameliorating” refers to medical management of a condition, disease, or disorder of a subject (e.g., patient), which may be therapeutic, prophylactic/preventative, or a combination treatment thereof.

An “effective amount” or a “therapeutically effective amount” may refer to an amount of therapeutic agent (e.g., an engineered IL2 polypeptide or engineered IL2 fusion polypeptide described herein) that provides a desired physiological change, such as an anti-cancer effect. The desired physiological change may, for example, be a decrease in symptoms of a disease, or a decrease in severity of a disease, or may be a reduction in the progression of a disease. With respect to cancer, the desired physiological changes may include, for example, tumor regression, a decreased rate of tumor progression, a reduced level of a cancer biomarker, reduced symptoms associated with cancer, a prevention or delay in metastasis, or clinical remission.

As used herein, the term “inhibit” refers the reduction of a specified activity (e.g., immune suppression or tumor growth). Unless specified otherwise, an activity can be considered inhibited if the activity is reduced by at least 10%, 20%, 30%, 40%, 50%, 60%, 70%, 80%, 90%, 95%, 98%, 99%, or 100%, as measured by the methods disclosed herein or known in the art.

“Checkpoint inhibitor” refers to an agent that reduces the activity of an immune checkpoint protein. A checkpoint inhibitor can be an antigen binding moiety that binds to and reduces activity of an immune checkpoint protein. Immune checkpoint proteins include, for example, programmed cell death protein 1 (PD-1 or CD279), programmed death-ligand 1 (PD-L1 or CD274), cytotoxic T-lymphocyte-associated antigen 4 (CTLA-4 or CD152), T-cell immunoglobulin mucin-3 (TIM3), Lymphocyte Activating 3 (LAG3 or CD223), B7-H2 (ICOSL or CD275), and B7-H3 (CD276). Examples of checkpoint inhibitors includes ipilimumab (an anti-CTLA-4 antibody), nivolumab (an anti-PD-1 antibody), and pembrolizumab (an anti-PD-1 antibody).

“Tumor infiltrating lymphocyte” or “TIL” refers to a lymphocyte that is isolated from tumor tissue, manipulated in vitro (e.g., stimulated using a cytokine such as interleukin-2), and then infused back into a patient so that the activating TIL returns to the tumor site and induces tumor regression.

“Tumor microenvironment inhibitor” refers to an agent that inhibits one or more conditions or cell types that promote tumor growth and are present in the local environment surrounding a tumor. For example, bevacizumab can inhibit the tumor microenvironment by reducing angiogenesis in a tumor microenvironment.

As used herein, PD-L1 (also known as “programmed death-ligand 1” or CD274 in humans) refers to the protein of UniProt Accession No. QOGN75 (human) and related isoforms and orthologs. As used herein, PD-1 (also known as “Programmed cell death protein 1,” “PDCD1,” or CD279) refers to the protein of UniProt Accession No. Q15116 (human) and related isoforms and orthologs.

Recombinant DNA, molecular cloning, and gene expression techniques used in the present disclosure are known in the art and described in references, such as Sambrook et al., Molecular Cloning: A Laboratory Manual, 3^{rd} Ed., Cold Spring Harbor Laboratory, New York, 2001, and Ausubel et al., Current Protocols in Molecular Biology, John Wiley and Sons, Baltimore, MD, 1999.

### Engineered Interleukin-2 Polypeptide

As noted above, IL2 polypeptides of the present disclosure include IL2Rβ reduced-binding agonists having an engineered IL2 receptor β (IL2Rβ) binding region 2. In some embodiments, the IL2Rβ binding region 2 comprises: X₁-X₂-X₃-X₄-X-5-X₆-X₇-X₈-X₉-X₁₀-X₁₁-X₁₂-X₁₃-X₁₄-X₁₅-X₁₆ (SEQ ID NO: 3), wherein
X₁ comprises a residue selected from R, S, L, N, F, K, or T;
X₂ comprises a residue selected from A, F, S, L, R, T, I, H, P, or N;
X₃ comprises a residue selected from K, R, T, S, I, or P;
X₄ comprises a residue selected from G, D, R, A, Q, H, N, Y, or E;
X₅ comprises a residue selected from I, P, T, S, K, F, V, or L;
X₆ comprises a residue selected from I, R, V, M, T, or L;
X₇ comprises a residue selected from A, R, M, I, S, N, G, or S;
X₈ comprises a residue selected from E, N, H, T, K, Y, S, L, V, D, or R;
X₉ comprises a residue selected from I, V, A, T, L, T, or M;
X₁₀ comprises a residue selected from N, G, V, Y, I, W, R, K, Q, A, D, S, or D;
X₁₁ comprises a residue selected from F, G, V, N, T, I, R, E, or A;
X₁₂ comprises a residue selected from I, S, R, V, P, G, T, L, M, F, or Y;
X₁₃ comprises a residue selected from V, I, F, D, P, H, A, V, or L;
X₁₄ comprises a residue selected from L, Q, R, E, P, K, H, W, F, or V;
X₁₅ comprises a residue selected from A, E, L, K, V, D, Y, R, or Q; and
X₁₆ comprises a residue selected from L or I.

For example, in some embodiments the engineered IL2 polypeptide comprises the amino acid sequence:

(SEQ ID NO: 45)
APASSSTKKTQLQLEHLLLDLQMILNGINNYKNPLLTDMLTRKFYM
PKKATELKHLQCLEEELKPLEEVLNLAQSKNFHL-X₁-X₂-X₃-X₄-
X-₅-X₆-X₇-X₈-X₉-X₁₀-X₁₁-X₁₂-X₁₃-X₁₄-X₁₅-X₁₆-KGSETTFM
CEYADETATIVEFLNRWITFSQSIISTLT,

wherein
X₁ comprises a residue selected from R, S, L, N, F, K, or T;
X₂ comprises a residue selected from A, F, S, L, R, T, I, H, P, or N;
X₃ comprises a residue selected from K, R, T, S, I, or P;
X₄ comprises a residue selected from G, D, R, A, Q, H, N, Y, or E;
X₅ comprises a residue selected from I, P, T, S, K, F, V, or L;
X₆ comprises a residue selected from I, R, V, M, T, or L;
X₇ comprises a residue selected from A, R, M, I, S, N, G, or S;
X₈ comprises a residue selected from E, N, H, T, K, Y, S, L, V, D, or R;
X₉ comprises a residue selected from I, V, A, T, L, T, or M;
X₁₀ comprises a residue selected from N, G, V, Y, I, W, R, K, Q, A, D, S, or D;
X₁₁ comprises a residue selected from F, G, V, N, T, I, R, E, or A;
X₁₂ comprises a residue selected from I, S, R, V, P, G, T, L, M, F, or Y;
X₁₃ comprises a residue selected from V, I, F, D, P, H, A, V, or L;
X₁₄ comprises a residue selected from L, Q, R, E, P, K, H, W, F, or V;
X₁₅ comprises a residue selected from A, E, L, K, V, D, Y, R, or Q; and
X₁₆ comprises a residue selected from L or I.

The engineered IL2 polypeptides disclose herein do not comprise not comprise a wild-type IL2Rβ binding region 2 motif, i.e., SEQ ID NO:2. The engineered IL2 polypeptides bind to IL2Rβ at a reduced affinity compared to a wild-type IL2 (SEQ ID NO:1) or an IL2 having a T3A and C125S substitution relative to SEQ ID NO:1, e.g., SEQ ID NO:171.

In some embodiments, the engineered IL2 polypeptide comprises a IL2Rβ binding region 2 selected from: RFKALIIEINFIVQLL (SEQ ID NO:4), RSRQLISNMNGIILKL (SEQ ID NO: 5), RLTHLRNVIGVILVQL (SEQ ID NO:6), LSLREPIGNIVTSVRE (SEQ ID NO:7), NRTDLVGDVNATIKAL (SEQ ID NO:8), RNKGILGDISNIVLAL (SEQ ID NO:9), RSREVVSRIDAIILEL (SEQ ID NO:10), RPRGLISDISNIVLAL (SEQ ID NO:11), RPRGLIGNISNIVLAL (SEQ ID NO:12), RPRGLIGDINNIVLAL (SEQ ID NO:13), RPKGLISNISNIVLAL (SEQ ID NO:14), RPKGLISDINNIVLAL (SEQ ID NO:15), RPKGLIGNINNIVLAL (SEQ ID NO:16), RNRGLISNISNIVLAL (SEQ ID NO:17), RNRGLISDINNIVLAL (SEQ ID NO:18), RNRGLIGNINNIVLAL (SEQ ID NO:19), RNKGLISNINNIVLAL (SEQ ID NO:20), RPRGLIGDISNIVLAL (SEQ ID NO:21), RPKGLISDISNIVLAL (SEQ ID NO:22), RPKGLIGNISNIVLAL (SEQ ID NO:23), RPKGLIGDINNIVLAL (SEQ ID NO:24), RNRGLISDISNIVLAL (SEQ ID NO:25), RNRGLIGNISNIVLAL (SEQ ID NO:26), RNRGLIGDINNIVLAL (SEQ ID NO:27), RNKGLISNISNIVLAL (SEQ ID NO:28), RNKGLISDINNIVLAL (SEQ ID NO:29), RNKGLIGNINNIVLAL (SEQ ID NO:30), RPRDLISDISNIVLAL (SEQ ID NO:31), RPRGLISDINNIVLAL (SEQ ID NO:32), RPRGLISDISVIVLAL (SEQ ID NO:33), RPRGLISDISNIVLEL (SEQ ID NO:34), RPRDLISDINNIVLAL (SEQ ID NO:35), RPRDLISDISVIVLAL (SEQ ID NO:36), RPRDLISDISNIVLEL (SEQ ID NO:37), RPRGLISDINVIVLAL (SEQ ID NO:38), RPRGLISDINNIVLEL (SEQ ID NO:39), RPRGLISDISVIVLEL (SEQ ID NO:40), RPKDLISNISNIVLAL (SEQ ID NO:41), RPKGLISNINNIVLAL (SEQ ID NO:42), RPKGLISNISVIVLAL (SEQ ID NO:43), RPKGLISNISNIVLEL (SEQ ID NO:44), RPKGLISNISVIVLEL (SEQ ID NO: 194), RPRGLISNISVIVLEL (SEQ ID NO:195), RPRDLISNISNIVLEL (SEQ ID NO: 196), RPKGLISNINNIVLEL (SEQ ID NO: 197), RPKGLISDINNIVLEL (SEQ ID NO: 198), RPRDLISRIDAIVLEL (SEQ ID NO: 199), RNRGLIGNINNIVLEL (SEQ ID NO: 200), RPKGLISEINNIVLEL (SEQ ID NO: 201), and RPKGLISRINNIVLEL (SEQ ID NO: 202).

In some embodiments, the engineered IL2 polypeptide comprises a IL2Rβ binding region 2 selected from: RFKALIIEINFIVQLL (SEQ ID NO:4), RSRQLISNMNGIILKL (SEQ ID NO:5), RLTHLRNVIGVILVQL (SEQ ID NO:6), RNKGILGDISNIVLAL (SEQ ID NO:9), RSREVVSRIDAIILEL (SEQ ID NO:10), RPRGLISDISNIVLAL (SEQ ID NO:11), RPRGLIGDINNIVLAL (SEQ ID NO:13), RPKGLISNISNIVLAL (SEQ ID NO:14), RPRGLIGDISNIVLAL (SEQ ID NO:21), RPKGLISDISNIVLAL (SEQ ID NO:22), RPKGLIGDINNIVLAL (SEQ ID NO:24), RNKGLISNISNIVLAL (SEQ ID NO:28), RNKGLISDINNIVLAL (SEQ ID NO:29), RPKGLISNISVIVLEL (SEQ ID NO: 194), RPRGLISNISVIVLEL (SEQ ID NO:195), RPRDLISNISNIVLEL (SEQ ID NO: 196), RPKGLISNINNIVLEL (SEQ ID NO: 197), RPKGLISDINNIVLEL (SEQ ID NO: 198), RPRDLISRIDAIVLEL (SEQ ID NO: 199), RNRGLIGNINNIVLEL (SEQ ID NO: 200), RPKGLISEINNIVLEL (SEQ ID NO: 201), and RPKGLISRINNIVLEL (SEQ ID NO: 202).

In some embodiments, the engineered IL2 polypeptide has decreased affinity for IL2Rβ as compared to the wild-type IL2 or an IL2 having the sequence of SEQ ID NO:171. In certain embodiments, the binding of the engineered IL2 polypeptide to IL2Rβ has a K_{D} at least 10-fold less, at least 15-fold less, at least 20-fold less, at least 25-fold less, or at least 30-fold less than a wild-type IL2 or an IL2 having the sequence of SEQ ID NO:171. In some embodiments, the engineered IL2 polypeptide binds to IL2Rβ with a K_{D} at least 30-fold less than a wild-type IL2 or an IL2 having the sequence of SEQ ID NO:171. In some embodiments, the engineered IL2polypeptide has at least a 10-fold decrease, at least a 15-fold decrease, at least a 20-fold decrease, at least a 25-fold decrease, or at least a 30-fold decrease in affinity for IL2Rβ as compared to wild-type IL2 or an IL2 having the sequence of SEQ ID NO:171. Assays used to detect IL2 binding may include ELISA or surface plasmon resonance (SPR) detection.

In some embodiments, the engineered IL2 polypeptide has a decrease in affinity for IL2 receptor α (IL2Rα) as compared to wild-type IL2 or an IL2 having the sequence of SEQ ID NO:171. In certain embodiments, the engineered IL2 polypeptide has at least a 5% decrease, at least a 10% decrease, at least a 15% decrease, or at least a 20% decrease in affinity for IL2Rα as compared to wild-type IL2 or an IL2 having the sequence of SEQ ID NO:171. In some embodiments, the engineered IL2 polypeptide has no detectable binding to IL2Rα as compared to wild-type IL2 or an IL2 having the sequence of SEQ ID NO:171. Assays used to detect IL2 binding may include ELISA or SPR detection.

In some embodiments, the engineered IL2 polypeptide has a similar affinity for IL2Rα as compared to wild-type IL2. In certain embodiments, the engineered IL2 polypeptide has an affinity for IL2Rα that varies from the affinity of wild-type IL2 for IL2Rα by no more than ±20%, no more than ±15%, no more than ±10%, or no more than ±5%.

Some embodiments of the present disclosure provide an engineered IL2 polypeptide comprising an engineered IL2Rα binding region 1. The engineered IL2Rα binding region 1 can comprise a substitution selected from: a substitution at position K35, a substitution at R38, a substitution at F42, a substitution at Y45, or combinations thereof. In some embodiments, the engineered IL2 polypeptide binds to IL2Rα with at least 2-fold reduced binding kinetics as compared to wild-type IL2.

In some embodiments, the engineered IL2 polypeptide may comprise a substitution at position K35. In some embodiments, the substitution at position K35 comprises a non-basic residue. In some embodiments, the substitution at position K35 comprises an uncharged residue or an acidic residue. In some embodiments, the substitution at position K35 is selected from: K35G, K35L, K35S, K35V, K35D, K35E, and K35C.

In some embodiments, the engineered IL2 polypeptide comprises a substitution at position R38. In some embodiments, the substitution at position R38 comprises a non-basic residue. In some embodiments, the substitution at position R38 comprises an uncharged residue or an acidic residue. In some embodiments, the substitution at position R38 is selected from: R38V, R38D, R38E, R38S, R38I, R38A, R38Y, R38G, R38C, and R38N.

In some embodiments, the engineered IL2 polypeptide may comprise a substitution at position F42. In some embodiments, the substitution at position F42 comprises an uncharged residue. In some embodiments, the substitution at position F42 comprises a basic residue. In some embodiments, the substitution at position F42 is selected from: F42A, F42R, F42G, F42I, F42L, F42P and F42H.

In some embodiments, the engineered IL2 polypeptide may comprise a substitution at position Y45. In some embodiments, the substitution at position Y45 comprises an uncharged residue. In some embodiments, the substitution at position Y45 comprises an uncharged polar residue or an uncharged non-polar residue. In some embodiments, the Y45 substitution is Y45S, Y45P, Y45A, Y45V, Y45C, Y45T, and Y45F.

In some embodiments, the engineered IL2 polypeptide may comprise a substitution at position K35 and a substitution at position R38. In some embodiments, the engineered IL2 polypeptide comprises a K35G substitution and R38E substitution.

In some embodiments, the engineered IL2 polypeptide may comprise a substitution at position K35 and a substitution at position F42. In some embodiments, the engineered IL2 polypeptide comprises a K35S substitution and an F42G substitution.

In some embodiments, the engineered IL2 polypeptide may comprise a substitution at position K35, a substitution at position R38, and a substitution at position F42. In some embodiments, the engineered IL2 polypeptide comprises a K35L substitution, an R38D substitution, and an F42R substitution.

In some embodiments, the engineered IL2 polypeptide may comprise a substitution at position R38 and a substitution at position Y45S. In some embodiments, the engineered IL2 polypeptide comprises an R38D substitution and an Y45S substitution. In some embodiments, the engineered IL2 polypeptide comprises an R38V substitution and an Y45S substitution.

In some embodiments, the engineered IL2 polypeptide binds to IL2Rα with at least 10-fold reduced binding kinetics as compared to wild-type IL2.

In some embodiments, the engineered IL2 is further modified to include a T3A substitution. The T3A substitution may be made to the wild-type sequence. In addition, the T3A substitution may be made to an engineered IL2 sequence disclosed herein. In some embodiments, the engineered IL2 is further modified to include a C125S substitution. The C125S substitution may be made to the wild-type sequence. In addition, the C125S substitution may be made to an engineered IL2 sequence disclosed herein. In some instances, the engineered IL2 sequence further includes both a T3A and a C125S modification.

In some embodiments, the engineered IL2 polypeptide shares at least 80%, at least 85%, at least 88%, at least 90%, at least 92%, at least 95%, at least 96%, at least 97%, at least 98%, at least 99% or at least 100% sequence identity with the residues outside of the IL2Rβ binding region 2 (i.e., residues 1-80 and 97-133) of SEQ ID NO:1 and binds to IL2Rβ. In some embodiments, the engineered IL2 polypeptide shares at least 80%, at least 85%, at least 88%, at least 90%, at least 92%, at least 95%, at least 96%, at least 97%, at least 98%, at least 99% or at least 100% sequence identity with the residues outside of the IL2Rβ binding region 2 (i.e., residues 1-80 and 97-133) of SEQ ID NO:171 and binds to IL2Rβ.

In some embodiments, the engineered IL2 polypeptide has a sequence corresponding to any one of SEQ ID NOS:46-102, 147-169, and/or and 203-211. In certain embodiments, the engineered IL2 polypeptide shares at least 80%, at least 85%, at least 88%, at least 90%, at least 92%, at least 95%, at least 96%, at least 97%, at least 98%, at least 99% or at least 100% sequence identity with the residues outside of the IL2Rβ binding region 2 (i.e., residues 1-80 and 97-133) of any one of SEQ ID NOS:46-102, 147-169, and/or and 203-211 and binds to IL2Rβ.

In some embodiments, the engineered IL2 polypeptide is selected from any one of SEQ ID NOS:46-102, 147-169, and/or and 203-211, with a C-terminal histidine tag optionally included (or excluded). In some embodiments, the C-terminal histidine tag is replaced with a linker, such as a Gly-Ser linker.

### Engineered IL2 Fusion Polypeptides

Some embodiments of the present disclosure provide engineered IL2 fusion polypeptides. The engineered IL2 fusion polypeptide may include an engineered IL2 polypeptide as described herein (see, e.g., SEQ ID NOS:3-102, 147-169, 203-211 and Table 2, 5, and 7) and at least one additional molecule covalently attached to the engineered IL2 polypeptide via a peptide bond or other chemical linkage. In certain embodiments, the engineered IL2 fusion polypeptide include an engineered IL2 polypeptide that shares at least 80%, at least 85%, at least 88%, at least 90%, at least 92%, at least 95%, at least 96%, at least 97%, at least 98%, at least 99% or at least 100% sequence identity with the residues outside of the IL2Rβ binding region 2 (i.e., residues 1-80 and 97-133) of any one of SEQ ID NOS:46-102, 147-169, and/or 203-211 and binds to IL2Rβ. In some embodiments, the engineered IL2 fusion polypeptide comprises a first polypeptide sequence and a second polypeptide sequence, wherein the first polypeptide sequence comprises an engineered IL2 polypeptide as described herein. In some embodiments, the second polypeptide sequence of the engineered IL2 fusion polypeptide comprises a polypeptide having a length of 5-500 amino acids, 5-400 amino acids, 5-300 amino acids, 5-250 amino acids, 5-200 amino acids, 50-500 amino acids, 50-400 amino acids, 50-300 amino acids, 50-250 amino acids, or 50-200 amino acids. In some embodiments, the second polypeptide sequence of the engineered IL2 fusion polypeptide comprises a polypeptide having a length of at least 5, at least 20, at least 40, at least 60, at least 80, 100, at least 150, at least 200, at least 250, or at least 300 amino acids. Non-limiting examples of polypeptides that may comprise the second polypeptide sequence of the fusion protein include a Fc domain, antibody, antigen binding moiety, cytokine, half-life extending molecule, tag or marker polypeptide, targeting domain, transport molecule, immunotoxin (e.g., diphtheria toxin), NKG2D, linker sequence, chemically linked small molecule, nucleic acid, PEGylation, or any combination thereof. In some embodiments, the second polypeptide sequence comprises a ligand or a scaffold protein.

In certain embodiments, the engineered IL2 fusion polypeptide comprises an engineered IL2 polypeptide as described herein and an IgG heavy chain constant region (e.g., Fc domain). The IL2 fusion polypeptide comprising an IgG heavy chain constant region may be referred to herein as an IL2-Fc fusion protein or IL2-Fc fusion polypeptide. In some embodiments, the IgG heavy chain constant region is derived from an IgG antibody. Human IgG antibodies have several subclasses, including, but not limited to IgG1, IgG2, IgG3, and IgG4. In particular embodiments, the IgG heavy chain constant region is derived from a human IgG1 antibody or an IgG4 antibody. In some embodiments, the IgG heavy chain constant region has one or more substitutions that reduce effector function of the IgG heavy chain constant region. In certain embodiments, the IgG heavy chain constant region comprises, relative to a human IgG, one or more mutations selected from L234A, L235A, P329G, Y349C, S354C, T366S, T366W, L368A, F405K, K409A and Y407V, numbered according to the EU numbering system. In some embodiments, the IgG heavy chain constant region comprises the sequence of SEQ ID NO:137, or a sequence having at least 90%, at least 91%, at least 92%, at least 93%, at least 94%, at least 95%, at least 96%, at least 97%, at least 98%, or at least 99% sequence identity to SEQ ID NO:137. In some embodiments, the Fc domain comprises LALAPG mutations. LALAPG mutations refer to L234A, L235A, and P329G changes in the CH2-CH3 region of an IgG heavy chain constant region, e.g., IgG1 (see, e.g., Schlothauer et al., Protein Engineering, Design & Selection. 29(10):457-466, 2016; Lo et al., J. Biol. Chem. 292(9):3900-3908, 2017, hereby incorporated by reference). In some embodiments, the antibody IgG heavy chain constant region comprises knob mutations or hole mutations. Knob-into-hole mutations are modifications to the IgG heavy chain constant region that allow heterodimerization of IgG heavy chain constant region that comprise the knob and hole mutations, respectively. Knob or hole mutations allow for preferential heterodimer formation in vitro with low levels of homodimer contaminants. Knob-into-hole mutations are disclosed in Merchant et al. Nat. Biotechnol. 16:677-681, 1998; and Wei et al., Oncotarget. 8(31):51037-51049, 2017, which are hereby incorporated by reference. Examples of knob mutations include S354C, T366W and K409A mutations in an IgG heavy chain constant region. Examples of hole mutations include Y349C, T366S, L368A, F405K, and Y407V mutations in an IgG heavy chain constant region. In some embodiments, the IgG heavy chain constant region comprises LALAPG mutations and hole mutations or knob mutations. For example, in some embodiments, the IgG heavy chain constant region can comprise L234A, L235A, P329G, S354C, T366W and K409A mutations. In some embodiments, the IgG heavy chain constant region can comprise L234A, L235A, P329G, Y349C, T366S, L368A, F405K, and Y407V mutations. In certain embodiments, the IgG heavy chain constant region comprises the amino acid sequence of SEQ ID NO:123 or 138. In certain embodiments, a protein complex may be formed with a first polypeptide comprising a first antibody IgG heavy chain constant region comprising, relative to SEQ ID NO:137, one or more mutations selected from S354C, T366W and K409A and a second polypeptide that comprising a second IgG heavy chain constant region comprising, relative to SEQ ID NO:137, one or more mutations selected from Y349C, T366S, L368A, F405K and Y407V, numbered according to the EU numbering system. In certain embodiments, the protein complex comprises a first antibody IgG heavy chain constant region comprising, relative to SEQ ID NO:137, one or more mutations selected from L234A, L235A, P329G, S354C, T366W and K409A and a second antibody IgG heavy chain constant region comprising, relative to SEQ ID NO:137, one or more mutations selected from L234A, L235A, P329G, Y349C, T366S, L368A, F405K and Y407V, numbered according to the EU numbering system. In some embodiments, the IgG heavy chain constant region comprises at least one amino acid residue modification to increase serum half-life. Representative modifications to the IgG heavy chain constant region are described in U.S. Pat. Nos. 7,317,091; 8,735,545; 7,371,826; 7,670,600; and 9,803,023.

In some embodiments, the engineered IL2 fusion polypeptide includes an engineered IL2 polypeptide linked to a fusion partner, such as an IgG heavy chain constant region. A variety of linkers are known in the art and may be used to covalently link an engineered IL2 described herein to a fusion partner, such as an Fc region. By “linker”, “linker sequence”, herein is meant a molecule or group of molecules (such as a monomer or polymer) that connects two molecules and often serves to place the two molecules in a preferred configuration. The linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. Useful linkers include glycine-serine polymers (including, for example, (GS)n, (GSGGS)n, (GGGGS)n, and (GGGS)n, where n is an integer of at least one), glycine-alanine polymers, alanine-serine polymers, and other flexible linkers. In some embodiments, the fusion polypeptide is a bivalent fusion polypeptide. A bivalent fusion polypeptide may refer to a molecular complex that includes two copies of engineered IL2 polypeptides, which may be of the same sequence or different sequences. The molecular complex may be bound non-covalently. For example, a bivalent fusion polypeptide may include two Fc regions bound together by one or more disulfide bridges or by knobs-into-holes chemistry

In some embodiments, the fusion polypeptide is a monovalent fusion polypeptide, e.g., a monovalent engineered IL2-Fc fusion polypeptide. A monovalent fusion polypeptide refers to a fusion polypeptide that has one copy of an engineered IL2 polypeptide. In some embodiments, the fusion polypeptides disclosed herein comprise a sequence having at least 90%, at least 91%, at least 92%, at least 93%, at least 94%, at least 95%, at least 96%, at least 97%, at least 98%, at least 99%, or at least 100% sequence identity sequence identity to a sequence selected from SEQ ID NOS: 124-128 and/or 190-193.

In some embodiments, the monovalent engineered IL2-Fc fusion polypeptide forms a complex with at least one additional protein. In some embodiments, the monovalent engineered IL2-Fc fusion polypeptide complex includes a first protein comprising a fusion polypeptide as described herein, and a second protein that forms a dimer with the first protein. For example, the second protein can be an IgG heavy chain constant region (Fc domain). The second protein can be an IgG heavy chain constant region having the sequence of SEQ ID NO:123. In certain embodiments, the monovalent engineered IL2-Fc fusion polypeptide complex comprises first polypeptide sequence selected from SEQ ID NOS:124-128 and/or 190-193 and a second polypeptide having the sequence of SEQ ID NO:123. For example, the monovalent engineered IL2-Fc fusion polypeptide complex can comprise: SEQ ID NOS: 124 and 123; SEQ ID NOS:

125 and 123; SEQ ID NOS: 126 and 123; SEQ ID NOS: 127 and 123; SEQ ID NOS: 128 and 123; SEQ ID NOS: 190 and 123; SEQ ID NOS: 191 and 123; SEQ ID NOS: 192 and 123; and/or SEQ ID NOS: 193 and 123.

In some embodiments, the fusion polypeptide disclosed herein includes an antigen binding moiety. In some embodiments, the antigen binding moiety comprises an immunoglobulin, a Fab molecule, an scFv, a bi-specific T-cell engager, a diabody, a single domain antibody, or a VHH antibody (nanobody). An antigen binding moiety may bind, for example, PD-L1, PD-1, CTLA-4, TIM3, LAG3, B7-H2, B7-H3, carcinoembryonic antigen (CEA), GD2, CD20, CD4, CD8, or cellular marker. Examples of PD-L1 antigen binding moieties include atezolizumab, avelumab, and durvalumab. Examples of PD-1 antigen binding moieties include nivolumab and pembrolizumab. An example of a CEA antigen moiety is CH1A1A-2F. An example of a GD-2 antigen binding moiety is dinutuximab, and an example of a CD20 antigen binding moiety is rituximab.

In some embodiments, disclosed herein is a protein complex, comprising a first polypeptide that is a fusion polypeptide as disclosed herein and a second polypeptide or protein complex comprising an antigen binding moiety. In certain embodiments, the second polypeptide or protein complex comprises a polypeptide having the sequences of SEQ ID NOS:103 and 114 or SEQ ID NOS: 103 and 172. In some embodiments, the protein complex comprises polypeptides having the sequences of SEQ ID NOS: 103 and 114 or SEQ ID NOS: 103 and 172, and a polypeptide having a sequence selected from SEQ ID NOS:124-128 and/or 190-193. In some embodiments, the protein complex comprises polypeptides having the sequences of: SEQ ID NOS: 103, 114, and 124; SEQ ID NOS: 103, 114, and 125; SEQ ID NOS: 103, 114, and 126; SEQ ID NOS: 103, 114, and 127; SEQ ID NOS: 103, 114, and 128, SEQ ID NOS: 103, 172, and 190; SEQ ID NOS: 103, 172, and 191; SEQ ID NOS: 103, 172, and 192; or SEQ ID NOS: 103, 172, and 193.

In some embodiments, the at least one additional molecule of the fusion polypeptide is a cytokine. In some embodiments, the cytokine is selected from interleukin-2, interleukin-15, interleukin-7, interleukin-10, and C-C motif chemokine ligand 19 (CCL19). In some embodiments, the additional molecule of the fusion polypeptide is a second engineered IL2 polypeptide as described herein.

In some embodiments, the at least one additional molecule of the fusion polypeptide is a half-life extending molecule. In some embodiments, the half-life extending molecule comprises a half-life extending polypeptide. In some embodiments, the half-life extending polypeptide comprises an Fc domain, human serum albumin (HSA), an HSA binding molecule, or transferrin.

In some embodiments, the half-life extending molecule comprises poly-ethylene glycol (PEG) or polypropylene glycol (PPG).

### Bifunctional Proteins

In certain embodiments, disclosed herein are bifunctional fusion proteins. The bifunctional fusion protein can comprise an engineered IL2 polypeptide as disclose herein and an antigen-binding moiety. In some embodiments, the bifunctional fusion protein is a single protein sequence. In other embodiments, the bifunctional fusion protein is a heterodimer formed by at least two protein sequences. The bifunctional fusion protein can include an antigen-binding moiety that comprises an immunoglobulin, Fab molecule, an scFv, a diabody, a single domain antibody, or a VHH antibody. The antigen-binding moiety can bind, for example, antigens on the surface of a cell such as marker proteins and/or markers of disease. The antigen-binding moiety can bind, for example, PD-L1, PD-1, CTLA-4, TIM3, LAG3, B7-H2, B7-H3, CD4, CD8, or a cellular marker. Examples of PD-L1 antigen binding moieties include atezolizumab, avelumab, and durvalumab. Examples of PD-1 antigen binding moieties include nivolumab and pembrolizumab. In some embodiments, the bifunctional fusion protein can further comprise an IgG Fc domain. In some embodiments, the Fc domain is a human IgG heavy chain constant region. In some embodiments, the antibody heavy chain constant region is a human IgG1 heavy chain constant region. The IgG Fc domain can comprise a wild-type antibody constant region or a modified antibody constant region. Examples of modified antibody constant regions are provided herein and include, for example, knob or hole mutations, and LALAPG mutations. In certain embodiments, the antibody heavy chain constant region comprises an amino acid sequence at least 90%, 91%, 92%, 93%, 94%, 95%, 96%, 97%, 98%, 99%, or 100% identical to SEQ ID NO:137. In certain embodiments, the antibody heavy chain constant region comprises, relative to SEQ ID NO:137, one or more mutations selected from L234A, L235A, P329G, Y349C, S354C, T366S, T366W, L368A, F405K, K409A and Y407V, numbered according to the

EU numbering system. In some embodiments, the antibody heavy chain constant region comprises an amino acid sequence of SEQ ID NO:123 or SEQ ID NO:138.

The antigen-binding moiety that binds PD-L1 can be any of the anti-PD-L1 antigen-binding moiety, antibodies, and/or scFv sequences disclosed herein. In some embodiments, the antigen-binding moiety that binds PD-L1 can comprise a Fab formed by an antibody heavy chain and light chain. In some embodiments, the antigen-binding moiety that binds PD-L1 can comprise a scFv.

In certain embodiments, the bifunctional fusion protein comprises an antigen-binding moiety that binds PD-L1 comprising: (i) a heavy chain variable domain (VH) comprising a CDR1 comprising the sequence of SEQ ID NO: 141; a CDR2 comprising the sequence of SEQ ID NO: 142; and a CDR3 comprising the sequence of SEQ ID NO: 143; and (ii) a light chain variable domain (VL) comprising a CDR1 comprising the sequence of SEQ ID NO: 144; a CDR2 comprising the sequence of SEQ ID NO: 145; and CDR3 comprising the sequence of SEQ ID NO: 146. In certain embodiments, the antigen-binding moiety that binds PD-L1 comprises a light chain and a heavy chain, wherein the light chain comprises or consists of an amino acid sequence at least 90%, 91%, 92%, 93%, 94%, 95%, 96%, 97%, 98%, 99%, or 100% identical to SEQ ID NO:103 and the heavy chain comprises or consists of an amino acid sequence at least 90%, 91%, 92%, 93%, 94%, 95%, 96%, 97%, 98%, 99%, or 100% identical to SEQ ID NO:104. In some embodiments, the bifunctional fusion protein has a sequence least 90%, 91%, 92%, 93%, 94%, 95%, 96%, 97%, 98%, 99%, or 100% identical to and one of SEQ ID NOS:106-113 and/or SEQ ID NOS:173-177. In some embodiments, the bifunctional fusion protein forms a heterodimer with a light chain comprising a polypeptide having the sequence of SEQ ID NO:103.

In some embodiments, disclosed herein is a protein complex (e.g., immunoconjugate) comprising a bifunctional fusion protein disclosed herein and a second antigen binding moiety. In some embodiments, the bifunctional fusion protein and the second antigen binding moiety have the same antigen binding moiety sequences. In some embodiments, the second antigen-binding moiety binds PD-L1. In some embodiments, the second antigen-binding moiety comprises: (i) a heavy chain variable domain (VH) comprising a CDR1 comprising the sequence of SEQ ID NO: 141; a CDR2 comprising the sequence of SEQ ID NO: 142; and a CDR3 comprising the sequence of SEQ ID NO: 143; and (ii) a light chain variable domain (VL) comprising a CDR1 comprising the sequence of SEQ ID NO: 144; a CDR2 comprising the sequence of SEQ ID NO: 145; and CDR3 comprising the sequence of SEQ ID NO: 146. In certain embodiments, the second antigen-binding moiety that binds PD-L1 comprises a light chain and a heavy chain, wherein the light chain comprises or consists of an amino acid sequence at least 90%, 91%, 92%, 93%, 94%, 95%, 96%, 97%, 98%, 99%, or 100% identical to SEQ ID NO:103 and the heavy chain comprises or consists of an amino acid sequence at least 90%, 91%, 92%, 93%, 94%, 95%, 96%, 97%, 98%, 99%, or 100% identical to SEQ ID NO:104. In some embodiments, the protein complex comprises polypeptides having the sequences of: SEQ ID NOS:104, 106, and 103; SEQ ID NOS:104, 107, and 103; SEQ ID NOS:104, 108, and 103; SEQ ID NOS:104, 109, and 103; SEQ ID NOS:104, 110, and 103; SEQ ID NOS:104, 111, and 103; SEQ ID NOS:104, 112, and 103; SEQ ID NOS:104, 113, and 103; SEQ ID NOS:172, 173, and 103; SEQ ID NOS:172, 174, and 103; SEQ ID NOS:172, 175, and 103; SEQ ID NOS:172, 176, and 103; or SEQ ID NOS:172, 177, and 103.

In some embodiments, the bifunctional fusion protein comprises an antigen-binding moiety binds PD-1. Examples of PD-1 antigen binding moieties include nivolumab and pembrolizumab. In some embodiments the bifunctional fusion protein comprises a sequence selected from the group comprising or consisting of SEQ ID NOS:118-122 or 180-189. The bifunctional fusion protein may further comprise a light chain sequence comprising a polypeptide having the sequence of SEQ ID NO:117 or SEQ ID NO:178. The bifunctional fusion protein may be included in a protein complex (e.g., an immunoconjugate). In some embodiments, the protein complex may comprise polypeptides having the sequences of: SEQ ID NOS: 116, 117, and 118; SEQ ID NOS: 116, 117, and 119; SEQ ID NOS: 116, 117, and 120; SEQ ID NOS: 116, 117, and 121; or SEQ ID NOS: 116, 117, and 122. In some embodiments, the protein complex may comprise polypeptides having the sequences of: SEQ ID NOS: 178, 179, and 180; SEQ ID NOS: 178, 179, and 181; SEQ ID NOS: 178, 179, and 182; SEQ ID NOS: 178, 179, and 183; SEQ ID NOS: 178, 179, and 184; SEQ ID NOS: 178, 179, and 185; SEQ ID NOS: 178, 179, and 186; SEQ ID NOS: 178, 179, and 187; SEQ ID NOS: 178, 179, and 188; or SEQ ID NOS: 178, 179, and 189.

In some embodiments, disclosed herein is a protein complex comprising polypeptides having the sequences of: SEQ ID NOS: 133, 134, and 135; or SEQ ID NOS: 133, 134, and 136.

### Vectors and Methods of Making Engineered IL2 Polypeptides

In further embodiments, provided is a polynucleotide encoding an engineered IL2, fusion polypeptide, protein complex, or bifunctional fusion protein described above and herein. In some embodiments, the polynucleotide comprises a nucleic acid sequence encoding a polypeptide or a combination of polypeptides selected from the group consisting of SEQ ID NOS:3-169, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence encoding a polypeptide or a combination of polypeptides selected from the group consisting of SEQ ID NOS:3-211. In some embodiments, the polynucleotide or polynucleotides are selected from the group consisting of DNA, cDNA, RNA or mRNA. Further provided is an expression cassette or multiple expression cassettes comprising one or more regulatory sequences operably linked to the polynucleotide or polynucleotides described above and herein.

In some embodiments, provided is a vector comprising the polynucleotide or polynucleotides, or an expression cassette, as described herein. In some embodiments, the vector is a plasmid vector or a viral vector. In some embodiments, the viral vector comprises a DNA virus or an RNA virus. In some embodiments, the viral vector is from a viral family selected from the group consisting of: Adenoviridae (e.g., Adenovirus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Herpesvirus, e.g., HSV-1), Parvoviridae (e.g., Parvovirus H1), Reoviridae (e.g., Reovirus), Retroviridae (e.g., Lentivirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesicular stomatitis virus (VSV)), Togaviridae (e.g., Alphavirus, Sindbis virus) and Enteroviridae (e.g., Echovirus). Further provided is a lipid nanoparticle (LNP), comprising the polynucleotide or polynucleotides, an expression cassette, or a vector, described above and herein.

Engineered IL2 polypeptides and/or engineered IL2 fusion polypeptides can be prepared by genetic or chemical methods well known in the art and by the methods disclosed in the Examples below. Genetic methods may include, for example, site-specific mutagenesis of the DNA sequence encoding the polypeptide, PCR, and gene synthesis. The intended nucleotide changes can be verified by sequencing. The nucleotide sequence of native IL2 has been described by Taniguchi et al. (Nature 302, 305-10 (1983)) and a nucleic acid encoding native human IL2 is available from, for example, American Type Culture Collection (Rockville Md.).

Engineered IL2 polypeptides or engineered IL2 fusion polypeptides may be obtained, for example, by recombinant production or solid-state peptide synthesis. For recombinant production, a polynucleotide encoding an engineered IL2 polypeptide or engineered IL2 fusion polypeptide can be isolated and inserted into one or more vectors for cloning and/or expression in a host cell. Such polynucleotides may be readily isolated and sequenced by conventional procedures. In certain embodiments, a vector, such as an expression vector, comprising one or more of the polynucleotides of the instant disclosure is provided. Methods which are well known to those skilled in the art can be used to construct expression vectors containing the coding sequence engineered IL2 polypeptide or engineered IL2 fusion polypeptide along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL (FOURTH EDITION), Cold Spring Harbor Laboratory, N.Y. (2012); and Ausubel et al., CURRENT PROTOCOLS IN MOLECULAR BIOLOGY, Greene Publishing Associates and Wiley Interscience, N.Y (1993). The expression vector can be part of a plasmid, virus, or may be a nucleic acid fragment. The expression vector includes an expression cassette into which the polynucleotide encoding engineered IL2 polypeptide or engineered IL2 fusion polypeptide (i.e., the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the disclosed herein may encode one or more polyproteins, which are post- or co-translationally separated into the final proteins via proteolytic cleavage. In addition, a vector, polynucleotide, or nucleic acid of the instant disclosure may encode heterologous coding regions, either fused of unfused to a first or second polynucleotide encoding the polypeptides disclosed herein, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (e.g., Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g., promoters inducible by tetracycline). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present disclosure may be associated with additional coding regions that encode secretory or signal peptides, which direct the secretion of a polypeptide encoded by a polynucleotide of the present disclosure. For example, if secretion of the engineered IL2 polypeptide or engineered IL2 fusion polypeptide is desired, DNA encoding a signal sequence may be placed upstream of the nucleic acid encoding the mature amino acids of the engineered IL2 polypeptide or engineered IL2 fusion polypeptide. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. For example, native human IL2 is translated with a 20 amino acid signal sequence at the N-terminus of the polypeptide, which is subsequently cleaved off to produce mature, 133 amino acid human IL2. In some embodiments, the native signal peptide, e.g., the IL2 signal peptide or an immunoglobulin heavy chain or light chain signal peptide is used, or a functional derivative of that sequence that retains the ability to direct the secretion of the polypeptide that is operably associated with it. In some embodiments, the signal peptide comprises or consists of SEQ ID NO:212.

In some embodiments, a polynucleotide encoding the engineered IL2 polypeptide or engineered IL2 fusion polypeptide further includes a DNA sequence encoding a sequence to facilitate purification (e.g., a histidine tag) or for labeling the engineered IL2 polypeptide or engineered IL2 fusion polypeptide within or at the ends of the polynucleotide encoding the engineered IL2 polypeptide or engineered IL2 fusion polypeptide.

In certain embodiments, a host cell comprising one or more polynucleotides encoding an engineered IL2 polypeptide or engineered IL2 fusion polypeptide is provided. In certain embodiments, the host cell comprises one or more vectors encoding the engineered IL2 polypeptide or engineered IL2 fusion polypeptide. The host cell can be any kind of cellular system that can be used to generate the engineered IL2 polypeptide or engineered IL2 fusion polypeptide. Such cells may be transfected or transduced as appropriate with the particular expression vector encoding the engineered IL2 polypeptide or engineered IL2 fusion polypeptide, and large quantities of vector containing cells can be grown for seeding large scale fermenters to obtain sufficient quantities of encoding the engineered IL2 polypeptide or engineered IL2 fusion polypeptide for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a polypeptide with a partially or fully human glycosylation pattern. Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or lymphoid cell (e.g., Y0, NS0, Sp20 cell).

Standard technologies are known in the art to express foreign genes in these systems. Cells expressing an engineered IL2 polypeptide fused to either the heavy or the light chain of an antigen binding moiety, such as an antibody, may be engineered so as to also express the other of the antibody chains such that the expressed engineered IL2 fusion polypeptide comprises an antibody that has both a heavy and a light chain.

In some embodiments, a method of producing an engineered IL2 polypeptide, engineered IL2 fusion polypeptide, or protein complex described herein is provided. In some embodiments, the method comprises culturing a host cell comprising a polynucleotide encoding an engineered IL2 polypeptide, engineered IL2 fusion polypeptide, or protein complex, as provided herein, under conditions suitable for expression of the engineered IL2 polypeptide, engineered IL2 fusion polypeptide, or protein complex, and optionally recovering and/or purifying the engineered IL2 polypeptide, engineered IL2 fusion polypeptide, or protein complex from the host cell (or host cell culture medium, for example, if the host cell secretes the polypeptide).

In some embodiments, disclosed herein is a transgenic feeder cell comprising an engineered IL2 polypeptide, a fusion polypeptide, a protein complex, a bifunctional fusion protein, a polynucleotide, or a vector disclosed herein. Example of feeder cells include K562 cells, 3T3 cells, fibroblasts (e.g., MEF), or an antigen presenting cell.

In some embodiments, disclosed herein is a method of in vitro or ex vivo expansion of immune cells, comprising contacting a population of immune cells with an effective amount of an engineered IL2 polypeptide, a fusion polypeptide, a protein complex, or a bifunctional fusion protein disclosed herein, under conditions sufficient to promote expansion the population of immune cells, and cultivating the population of immune cells for a time sufficient to at least double the number of immune cells. For example, the conditions sufficient to promote expansion may include one or more agents or ligands that are capable of activating an intracellular signaling domain of a TCR complex, such as an anti-CD3 antibody or binding domain, an anti-CD28 antibody or binding domain, or a combination thereof. In some embodiments, the method of expanding immune cells further comprises cultivating the population of immune cells with an IL-7, IL-15, IL-21, or any combination thereof. In some embodiments, the population of immune cells is cultivated in the presence of a population of feeder cells, such as the transgenic feeder cells disclosed herein.

### Pharmaceutical Compositions

Provided herein are pharmaceutical compositions comprising an engineered IL2 polypeptide, engineered IL2 fusion polypeptide, bifunctional fusion protein, protein complex, polynucleotide, vector, or cell as described herein and a pharmaceutically acceptable diluent(s), excipient(s), or carrier(s). In some embodiments, the pharmaceutical compositions comprise an engineered IL2 polypeptide, engineered IL2 fusion polypeptide, bifunctional fusion protein, protein complex, polynucleotide, vector, or cell as disclosed herein and an additional therapeutic agent (e.g., combination therapy). Non-limiting examples of such therapeutic agents are described herein below. The pharmaceutical compositions may be formulated in a conventional manner using one or more pharmaceutically acceptable carriers comprising excipients and auxiliaries which facilitate processing of the engineered IL2 polypeptide, engineered IL2 fusion polypeptide, bifunctional fusion protein, protein complex, polynucleotide, vector, or cell into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). Examples of IL-2 compositions are described in U.S. Pat. Nos. 4,604,377 and 4,766,106, which are incorporated by reference herein.

As used herein, “pharmaceutically acceptable carrier” and “physiologically acceptable carriers” are used interchangeably and include any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art and are molecular entities and compositions that are generally non-toxic to recipients at the dosages and concentrations employed, i.e., do not produce an adverse, allergic or other untoward reaction when administered to an animal, such as, for example, a human, as appropriate (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical composition may comprise different types of carriers depending on whether it is to be administered in solid, liquid or aerosol form, and whether it need to be sterile for such routes of administration as injection. Engineered IL2 polypeptides or engineered IL2 fusion polypeptides as describe herein (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the engineered IL2 polypeptides or engineered IL2 fusion polypeptides describe herein.

Provided herein is transgenic immune cell that expresses an engineered IL2 polypeptide, a fusion polypeptide, a protein complex, a bifunctional fusion protein, a polynucleotide, or a vector disclosed herein. In some embodiments, the transgenic immune cell is a CD4+ T cell, a CD8+ T cell, a γδ T cell, a NK cell, a T regulatory cell, or any combination thereof. In some embodiments, the transgenic immune cell further comprises a chimeric antigen receptor (CAR).

In some embodiments, the CAR comprises an antigen binding domain that is capable of binding targets/antigens produced by a cancer cell. For example, CAR comprises an antigen binding domain that is capable of binding an antigen such CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGaip(1-4)bDGicp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGicp(1-1)Cer); transglutaminase 5 (TGS5); high molecular weight-melanoma-associated antigen (HMWMAA); O-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1)

In some embodiments, the transgenic immune cell produces the engineered IL2 polypeptide, fusion polypeptide, protein complex, or bifunctional fusion protein, which is secreted by the transgenic immune cell. In other embodiments, the engineered IL2 polypeptide, fusion polypeptide, protein complex, or bifunctional fusion protein comprises a transmembrane domain or cell-surface anchor molecule is expressed on and/or localized to the surface of the cell. In some embodiments, the transgenic immune cell is an armored CAR T cell.

### Methods of Treatment and Use

In some embodiments of the present disclosure provided herein are methods of modulating an immune response in a subject in need thereof, comprising administering to the subject a therapeutically effective amount of an engineered IL2 polypeptide, an engineered IL2 fusion polypeptide, bifunctional fusion protein, protein complex, polynucleotide, vector, cell, or a pharmaceutical composition thereof as described herein (see, e.g., SEQ ID NOS:3-102, 147-169, 203-211, and Table 2, 5, and 7). In certain embodiments, modulating the immune response includes at least one of: enhancing effector T cell activity, enhancing NK cell activity, and suppressing regulatory T cell activity. In some embodiments of the present disclosure provided herein engineered IL2 polypeptides as described herein, fusion polypeptides as described herein, a protein complexes as described, bifunctional fusion protein, polynucleotide, vector, cell, and/or pharmaceutical compositions as described herein, for use in a method of modulating an immune response in a subject in need thereof.

In some embodiments of the present disclosure is a method of treating a disease or condition in a subject in need thereof, comprising administering to the subject a therapeutically effective amount of an engineered IL2 polypeptide, an engineered IL2 fusion polypeptide, a protein complex, a bifunctional fusion protein, polynucleotide, vector, cell, or a pharmaceutical composition thereof as described herein. In some embodiments, the engineered IL2 polypeptides, fusion polypeptides, protein complex, a bifunctional fusion protein, a polynucleotide, a vector, a cell and/or a pharmaceutical composition as described herein, for use in a method of treating a subject for a disease. Non-limiting examples of diseases or condition contemplated in the method include proliferative disorders, such as cancer and immunosuppression.

In some embodiments, is a method of treating a proliferative disorder comprising administering to the subject a therapeutically effective amount of an engineered IL2 polypeptide, an engineered IL2 fusion polypeptide, a protein complex, or a pharmaceutical composition thereof as described herein. In some embodiments, the proliferative disorder is cancer. In some embodiments, the cancer comprises breast cancer, pancreatic cancer, lung cancer, glioblastoma, renal cell carcinoma, lymphoma, leukemia, head & neck cancer, liver cancer, gastric cancer, colon/colorectal cancer, renal cancer, bladder cancer, or melanoma. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, glioblastoma, prostate cancer, hematological malignancies, skin cancer, squamous cell carcinoma, skin cancer, melanoma, bone cancer, renal cell carcinoma, and kidney cancer. Examples of hematological malignancies include leukemia, lymphoma (e.g., non-Hodgkin lymphoma or Hodgkin lymphoma), multiple myeloma, myelodysplastic syndrome, myeloproliferative neoplasms (e.g., essential thrombocythemia, myelofibrosis, and polycythemia vera). Also included are pre-cancerous conditions or lesions and cancer metastases. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Similarly, other cell proliferation disorders can also be treated, such as hypergammaglobulinemia, lymphoproliferative disorders, paraproteinemias, purpura, sarcoidosis, Sezary Syndrome, Waldenstron's Macroglobulinemia, Gaucher's Disease, histiocytosis, amyloidosis, and aplastic anemia, and any other cell proliferation disease, besides neoplasia, located in an organ system listed above.

In some embodiments, the method of treatment or modulating the immune response further comprises administering to the subject a therapeutically effective amount of at least one additional therapeutic agent (e.g., a combination therapy). In certain embodiments, the additional therapeutic agent is an anti-cancer agent. Examples of anti-cancer agents include checkpoint inhibitors (e.g., anti-PD1 antibodies), chemotherapeutic agents, agents that inhibit a tumor microenvironment, cancer vaccines (e.g., Sipuleucel-T), oncolytic viruses (e.g., talimogene laherparepvec), immune cells expressing a chimeric antigen receptor (CAR), and tumor infiltrating lymphocytes (TIL). In certain embodiments, the additional therapeutic agent is a molecule including an antigen binding moiety. In certain specific embodiments, the antigen binding moiety is selected from a single domain antibody, a Fab molecule, an scFv, a diabody, a nanobody, a bi-specific T cell engager, or an immunoglobulin. In certain embodiments, the antigen binding moiety is specific to a tumor antigen (e.g., carcinoembryonic antigen, fibroblast activation protein-α, CD20) or a check point protein (e.g., CTLA-4, PD-1 or PD-L1). In some embodiments, the additional therapeutic agent is a vaccine, gene therapy, cellular therapy, or any combination thereof. In some embodiments, the additional therapeutic agent comprises an immune cell expressing a chimeric antigen receptor, an immune cell expressing an engineered T cell receptor, or a tumor infiltrating lymphocyte. In certain embodiments, the engineered IL2 polypeptide, engineered IL2 fusion polypeptide, or protein complex may be encoded by a polynucleotide transfected, transduced, or otherwise introduced into the immune cell that expresses the chimeric antigen receptor, the immune cell expressing an engineered T cell receptor, or the tumor infiltrating lymphocyte. In such embodiments, the immune cell may be an armored chimeric antigen receptor-expressing cell. In certain embodiments, the immune cell is an T cell (e.g., CD4 and/or CD8 T cell) and/or an NK cell. The polynucleotide may additionally encode a secretion signal (e.g., the native IL2 signal sequence or a signal sequence derived from another protein) directly upstream of the engineered IL2 polypeptide coding sequence, to allow the cell to secrete the engineered IL2 polypeptide or engineered IL2 fusion polypeptide.

Suitable routes of administration include, but are not limited to, intravenous, parenteral, transdermal, oral, rectal, aerosol, ophthalmic, pulmonary, transmucosal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, an engineered IL2 polypeptide, IL2 fusion polypeptide, or protein complex is administered systemically. In certain embodiments, an engineered IL2 polypeptide, engineered IL2 fusion polypeptide, or protein complex as described herein is administered in a local rather than systemic manner, for example, via injection of the engineered IL2 polypeptide, engineered IL2 fusion polypeptide, or protein complex directly into an organ, tissue, or tumor. In some embodiments, long acting formulations are administered by implantation (for example subcutaneously or intramuscularly) or by intramuscular injection. Furthermore, in some embodiments, the drug is delivered in a targeted drug delivery system, for example, in a liposome coated with organ-specific or cell-specific antibody. In such embodiments, the liposomes are targeted to and taken up selectively by the organ. In some embodiments, the engineered IL2 polypeptide, engineered IL2 fusion polypeptide, or protein complex as described herein is provided in the form of a rapid release formulation, in the form of an extended or sustained release formulation, in the form of an intermediate release formulation, or in the form of a depot preparation. In some embodiments, the engineered IL2 polypeptide, engineered IL2 fusion polypeptide, or protein complex described herein is administered topically.

The appropriate dosage of an engineered IL2 polypeptide engineered IL2 fusion polypeptide, or protein complex (used alone or in combination with one or more other additional therapeutic agents) will depend on the type of disease or condition, the route of administration, body weight of the subject, severity and progression of the disease, whether the polypeptide is administered for preventive or therapeutic purposes, previous or concurrent therapeutic interventions, the subject's clinical history and response to the engineered IL2 polypeptide, engineered IL2 fusion polypeptide, or protein complex, and the discretion of the attending physician. The practitioner responsible for administration will be able to determine the concentration of active ingredient(s) in a composition and appropriate dosing for the subject to be treated. Various dosing schedules including but not limited to single or multiple administrations over various time-points, bolus administration, and pulse infusion are contemplated herein.

A single administration of an engineered IL2 polypeptide may range from about 50,000 IU/kg to about 1,000,000 IU/kg or more of the engineered IL2 polypeptide. This may be repeated several times a day (e.g., 2-4 times per day), for several days (e.g., 3-5 consecutive days) and then may be repeated one or more times following a period of rest (e.g., 7-14 days). Thus, a therapeutically effective amount may comprise only a single administration or many administrations over a period of time (e.g., about 10-30 individual administrations of about 600,000 IU/kg of IL2 each given over about a 5-20 day period). When administered in the form of a fusion polypeptide or protein complex, a therapeutically effective of the engineered IL2 fusion polypeptide or protein complex may be lower than a non-fusion engineered IL2 polypeptide (e.g., 10,000 IU/kg to about 600,000 IU/kg). Similarly, the engineered IL2 fusion polypeptide may be administered to the patient at one time or over a series of treatments as described above.

In certain embodiments, the daily dosage of the engineered IL2 polypeptide, engineered IL2 fusion polypeptide, or protein complex ranges from about 1 μg/kg to about 100 mg/kg or more. For repeated administrations over several days or longer, depending on the condition, the treatment may be sustained until a desired suppression of disease symptoms occurs (e.g., tumor shrinkage). In some embodiments, a single dose of an engineered IL2 polypeptide, engineered IL2 fusion polypeptide, or protein complex is in the range from about 0.005 mg/kg to about 10 mg/kg. In some embodiments, a dose may be about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Such doses may be administered intermittently, e.g., 2-3 times per day, every week, or every three weeks. An initial higher loading dose, followed by one or more lower doses may be administered. However, other dosage regimens may be useful.

The engineered IL2 polypeptides and engineered IL2 fusion polypeptides described herein may be used in an amount effective to achieve the intended purpose. For use to treat or prevent a disease condition, the engineered IL2 polypeptides or engineered IL2 fusion polypeptides, or pharmaceutical compositions thereof, are administered in a therapeutically effective amount. Determination of a therapeutically effective amount is within the capabilities of those of skill in the art, especially in light of the details provided herein.

For systemic administration, a therapeutically effective amount can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. Administration to humans could readily be optimized by a person of ordinary skill in the art based on animal data. Dosage amount and interval may each be adjusted to provide plasma levels of engineered IL2 polypeptides and engineered IL2 fusion polypeptides which are sufficient to maintain therapeutic effect. Levels in plasma may be measured, for example, by HPLC.

### EXAMPLES

In the EXAMPLES provided below, the IL2 labeled as “WT-IL2” refers to an IL2 having the sequence of SEQ ID NO:171, which includes wild-type IL2Rα and IL2Rβ binding domains and T3A and C125S substitutions.

### Example 1

### Screening and Identification of IL2RB Reduced-Binding Agonists

The in vitro mRNA display technology was used to develop IL2Rβ reduced-binding agonists from three rationally designed IL2 mutagenic libraries. In brief, the IL2 mutant DNA libraries were transcribed into mRNA libraries and then translated into mRNA-IL2 mutant fusion libraries through covalent coupling with a puromycin linker. The libraries were then purified and coupled with the corresponding single strand cDNA to stabilize the cytokine fused mRNA libraries in the format of mRNA/cDNA hybrid, similar to the procedure reported in expired U.S. Pat. No. 6,258,558. The fusion libraries were first counter selected for multiple rounds with human IgGs (negative proteins) to remove nonspecific binders, then counter selected against recombinant human IL2Rβ-Fc protein captured on Protein G magnetic beads. A total of 2-4 rounds of selections with gradient concentration were performed. The IL2Rβ reduced binders were recovered and enriched by PCR amplification at each round of selection.

After 2-4 rounds of selection, the enriched IL2Rβ reduced-binding libraries were cloned into bacterial periplasmic expression vector pET22b and transformed into TOP10 competent E. coli cells. Each engineered IL2 molecule was engineered to have a C-terminus flag and 6×HIS tag for purification and assay detection. Clones from TOP10 cells were pooled and the miniprep DNA were prepared and subsequently transformed into E. coli Rosetta II strain for expression. Single clones were picked, grown and induced with 0.25 mM IPTG in 96-well plates for expression. The supernatant was collected after 16 to 24 hours induction at 30° C. for assays to identify binders.

Supernatants containing engineered IL2 mutants were assessed with sandwich ELISA assay to screen for IL2Rα binding. Briefly, 96-well plate was immobilized with human Fc and human IL2Rα, respectively, at a final concentration of 2 μg/mL in 1× PBS in total volume of 50 μL per well. The plate was incubated overnight at 4° C. followed by blocking with 200 μL of superblock per well for 1 hour. 100 μl of supernatant was added to both Fc and IL2Rα immobilized wells and incubated for 1 hour with shaking. The engineered IL2 mutant binding was detected by adding 50 μL of anti-Flag HRP diluted at 1:5000 in 1× PBST. In between each step, the plate was washed three times with 1× PBST in a plate washer. The plate was then developed with 50 μL of TMB substrate for five minutes and stopped by adding 50 μL of 2N sulfuric acid. The plate was read at OD450 nm using a Biotek plate reader and the binding and selectivity was analyzed.

Single clones were next screened for IL2Rβ binding. IL2Rβ binding screening ELISA was developed for the identification of individual engineered IL2 mutant. Briefly, 96-well plate was immobilized with human Fc and human IL2Rβ, respectively, at a final concentration of 2 μg/mL in 1× PBS in total volume of 50 μL per well. The plate was incubated overnight at 4° C. followed by blocking with 200 μL of superblock per well for 1 hour. 100 μl of supernatant was added to both Fc and IL2Rβ immobilized wells and incubated for 1 hour with shaking. The engineered IL2 mutant binding was detected by adding 50 μL of anti-Flag HRP diluted at 1:5000 in 1× PBST. In between each step, the plate was washed three times with 1× PBST in a plate washer. The plate was then developed with 50 μL of TMB substrate for five minutes and stopped by adding 50 μL of 2N sulfuric acid. The plate was read at OD450 nm using a Biotek plate reader and the binding and selectivity was analyzed.

Clones showing positive IL2Rα binding but with reduced or negligible IL2Rβ-binding were identified from library 5 for further characterization. Multiple sequence alignment of the IL2Rβ binding loop 2 (IL2Rβ-BL2) revealed both highly conserved and highly varied amino acids as compared to IL2 WT, as well as clone sequences that were identified multiple times independently. No specific IL2Rβ-binding clones were identified from other libraries.

To further triage, the engineered IL2 clones with unique sequences were produced in E. coli. In short, the glycerol stock of each engineered agonist clone was inoculated into TB medium for overnight growth. The next day, cells from the overnight culture were inoculated into TB medium and grown to a cell density of OD₆₀₀ between 0.6-0.8. IPTG was added to a final concentration of 1 mM to induce the expression during culture at 30° C. overnight. The supernatant was collected by centrifugation. The agonists were purified by Ni-Sepharose (GE Healthcare) affinity column according to the manufacturer's protocol. The proteins were each stored in 1× PBS buffer for binding and functional analysis. Due to low expression of the most clones in E. coli, the activities were assessed by IL2Rα and IL2Rβ binding ELISA as described 10 above. Table 1 below listed the clones showing the corresponding OD450 values of IL2Rα and IL2Rβ receptor binding measured by anti-flag ELISA, respectively. Table 2 listed the sequences of engineered IL2 molecules with mutations in the region of amino acid from 81 to 96 (numbered based on matured WT-IL2 sequence).

TABLE 1
Receptor binding activities of engineered
IL2 clones produced in E. coli.

OD450 nm of single concentration receptor
binding ELISA

| Clones | IL2Rα | ILR2β |
|---|---|---|
| WT IL2 | | |
| 2021EP22-E11 | 0.086 | 0.148 |
| 2021EP22-F10 | 0.105 | 0.168 |
| 2021EP22-H09 | 0.251 | 0.389 |
| 2021EP21-C04 | 0.05 | 0.068 |
| 2021EP16-H06 | 1.255 | 0.105 |
| 2021EP22-E03 | 0.433 | 0.871 |
| 2021EP21-E03 | 0.528 | 0.583 |
| 2021EP22-H02 | 0.423 | 0.653 |
| 2021EP21-C03 | 0.079 | 0.143 |
| 2021EP16-E02 | 1.581 | 0.07 |
| 2021EP22-B04 | 0.171 | 0.524 |
| 2021EP16-D09 | 1.247 | 0.057 |
| 2021EP21-C10 | 0.361 | 1.119 |
| 2021EP16-E05 | 0.771 | 0.149 |
| 2021EP16-H02 | 0.857 | 0.532 |
| 2021EP16-E11 | 0.957 | 0.611 |
| 2021EP16-H09 | 0.715 | 0.56 |
| 2021EP16-C10 | 0.88 | 0.127 |
| 2021EP16-G10 | 1.084 | 0.337 |
| 2021EP16-H10 | 0.749 | 0.323 |
| 2021EP16-F05 | 0.635 | 0.299 |
| 2021EP16-C03 | 0.614 | 0.583 |
| 2021EP16-E07 | 0.624 | 0.505 |

TABLE 2
list of IL2 mutation sequences in region from 81 to 96 in comparison with the WT-IL2.

| | Original | | | | | | | | | | | | | | | | |
|---|---|---|---|---|---|---|---|---|---|---|---|---|---|---|---|---|---|
| Construct | Clones | 81 | 82 | 83 | 84 | 85 | 86 | 87 | 88 | 89 | 90 | 91 | 92 | 93 | 94 | 95 | 96 |
| WT-IL2 | | R | P | R | D | L | I | S | N | I | N | V | I | V | L | E | L |
| EP564 | 2021EP22-E11 | R | F | K | A | L | I | I | E | I | N | F | I | V | Q | L | L |
| EP565 | 2021EP22-F10 | R | S | R | Q | L | I | S | N | M | N | G | I | I | L | K | L |
| EP566 | 2021EP22-H09 | R | L | T | H | L | R | N | V | I | G | V | I | L | V | Q | L |
| EP567 | 2021EP21-C04 | S | L | R | E | P | I | G | N | I | V | T | S | V | R | E | L |
| EP568 | 2021EP16-H06 | L | A | K | R | T | V | S | N | M | Y | V | R | V | E | E | L |
| EP569 | 2021EP22-E03 | R | R | K | E | S | I | N | H | M | I | V | V | F | P | V | L |
| EP570 | 2021EP21-E03 | S | S | S | G | L | L | G | D | V | W | V | I | V | L | D | L |
| EP571 | 2021EP22-H02 | R | P | S | E | L | M | A | T | M | R | V | P | V | Q | A | L |
| EP572 | 2021EP21-C03 | R | A | R | D | I | V | R | N | A | K | V | G | V | Q | E | L |
| EP573 | 2021EP16-E02 | N | R | T | D | L | V | G | D | V | N | A | T | I | K | A | L |
| EP574 | 2021EP22-B04 | F | T | K | D | L | T | S | T | M | Q | I | L | V | L | Q | L |
| EP575 | 2021EP16-D09 | R | N | K | G | I | L | G | D | I | S | N | I | V | L | A | L |
| EP576 | 2021EP21-C10 | L | P | R | N | K | I | S | K | T | K | V | M | V | H | E | L |
| EP577 | 2021EP16-E05 | K | A | I | A | F | I | S | D | L | K | V | R | V | L | D | L |
| EP578 | 2021EP16-H02 | T | T | R | D | V | V | M | T | M | A | R | I | D | L | E | L |
| EP579 | 2021EP16-E11 | R | P | S | Y | I | V | S | Y | T | N | V | F | P | L | V | L |
| EP580 | 2021EP16-H09 | R | I | R | D | L | L | N | K | I | N | E | L | H | R | Y | I |
| EP581 | 2021EP16-C10 | R | S | R | E | V | V | S | R | I | D | A | I | I | L | E | L |
| EP582 | 2021EP16-G10 | T | H | R | D | V | I | S | D | V | N | V | L | A | L | E | L |
| EP583 | 2021EP16-H10 | S | A | I | D | L | M | S | S | V | K | V | V | V | W | K | L |
| EP584 | 2021EP16-F05 | R | P | R | D | L | I | S | N | I | N | V | I | V | L | E | L |
| EP585 | 2021EP16-C03 | S | P | P | D | L | I | A | N | M | A | V | Y | F | V | E | L |
| EP586 | 2021EP16-E07 | R | P | R | D | S | R | R | L | I | S | V | I | V | F | R | L |

### Example 2

### Binding Activities of IL2RB Reduced-Binding Agonists Using Surface Plasmon Resonance

The binding kinetics of E. coli produced WT IL2 and engineered clones were screened by surface plasmon resonance technology with Biacore T200. The assay was run with Biacore T200 control software version 2.0. For each cycle, 1 μg/mL of human IL2Rβ or IL2Rα was captured for 60 seconds at flow rate of 10 μL/min on flow cell 2 in 1× HBSP buffer on Protein A sensor chip. 100 nM and 20 nM of HIS tag purified engineered IL2 mutant was injected onto both reference flow cell 1 and IL2Rβ or IL2Rα captured flow cell 2 for 150 seconds at flow rate of 30 μL/min followed by washing for 300 seconds. The flow cells were then regenerated with Glycine pH 2 for 60 seconds at a flow rate of 30 μL/mins. The kinetics data was analyzed with Biacore T200 evaluation software 3000. The specific binding response unit was derived from subtraction of binding to reference flow cell 1 from target flow cell 2.

WT IL2 and an IL2Rβ enhanced binding agonist EP596 (SEQ ID NO:170) were used to validate the binding protocols and was included in each run as a control. Binding activities for E. coli produced IL2 and mammalian produced IL2 are summarized in the Table 3.

TABLE 3
Summary of IL2R binding activities of
E. coli-produced IL2Rβ agonist clones.

Binding KD (nM) by SPR
analysis

| Construct | IL2 Clones | IL2Rα | ILR2β |
|---|---|---|---|
| WT-IL2 | WT-IL2 | 11.93 | 282.7 |
| EP596 | EP596 | ND | 1.03 |
| EP564 | 2021EP22-E11 | 17.99 | ND |
| EP565 | 2021EP22-F10 | 142.1 | ND |
| EP566 | 2021EP22-H09 | 522.9 | ND |
| EP567 | 2021EP21-C04 | 187.9 | ND |
| EP568 | 2021EP16-H06 | weak binding | ND |
| EP569 | 2021EP22-E03 | 47.82 | ND |
| EP570 | 2021EP21-E03 | 21.33 | ND |
| EP571 | 2021EP22-H02 | 116.8 | weak binding |
| EP572 | 2021EP21-C03 | 75.49 | weak binding |
| EP573 | 2021EP16-E02 | 55.15 | weak binding |
| EP574 | 2021EP22-B04 | 207.1 | ND |
| EP575 | 2021EP16-D09 | 98.25 | weak binding |
| EP576 | 2021EP21-C10 | 143.4 | weak binding |
| EP577 | 2021EP16-E05 | 955.8 | weak binding |
| EP578 | 2021EP16-H02 | 9339 | ND |
| EP579 | 2021EP16-E11 | ND | weak binding |
| EP580 | 2021EP16-H09 | ND | ND |
| EP581 | 2021EP16-C10 | 106.2 | weak binding |
| EP582 | 2021EP16-G10 | 151.1 | weak binding |
| EP583 | 2021EP16-H10 | weak binding | weak binding |
| EP584 | 2021EP16-F05 | weak binding | weak binding |
| EP585 | 2021EP16-C03 | 4264 | weak binding |
| EP586 | 2021EP16-E07 | ND | weak binding |

Due to low expression of agonists in E. coli, clones with positive IL2Rα binding are indicative of reasonable expression of the clones in the system.
Weak binding: observable weak sensorgram signal (Rmax ≥ 5 RU) in SPR.
ND: not detectible binding (Rmax < 5RU).

### Example 3

### Characterization of Mammalian Cell Produced IL2RB Reduced-Binding Agonists

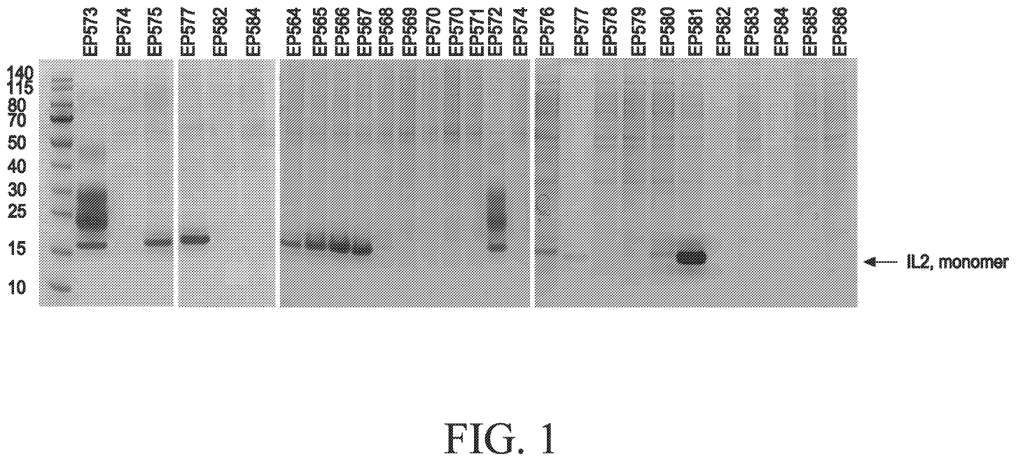

For production of the IL2Rβ reduced-binding agonists in mammalian cells, the DNA sequence corresponding to the amino-acid sequence was codon optimized, synthesized and subcloned into pCDNA3.4 (Invitrogen). Points mutations of F35L, R38D and F42R were introduced to each of the engineered IL2 sequence in order to reduce IL2Rα binding activities. Each engineered IL2 polypeptide was expressed transiently in ExpiHEK293-F cells in free style system (Invitrogen) according to standard protocol. The cells were grown in the above conditions for seven days before harvesting. The supernatant was collected by centrifugation and filtered through a 0.2 μm PES membrane. The agonists were first purified by Ni Sepharose Excel resin column (GE Healthcare) and buffer exchanged to PBS pH 7.4 with 300 mM NaCl by 7 k Da Zeba columns. Each polypeptide was then concentrated to 1 mL and purified by a Superdex 200 Increase 10/300 GL column (GE Healthcare) to homogeneity. The monomeric peak fractions were pooled and concentrated. The proteins were stored in 1× PBS/300 nM NaCl buffer for binding, functional and mechanism analysis. The example of SDS-PAGE (sodium dodecyl sulfate-polyacrylamide gel electrophoresis) results of individual IL2 after Ni column purification was illustrated in FIG. 1.

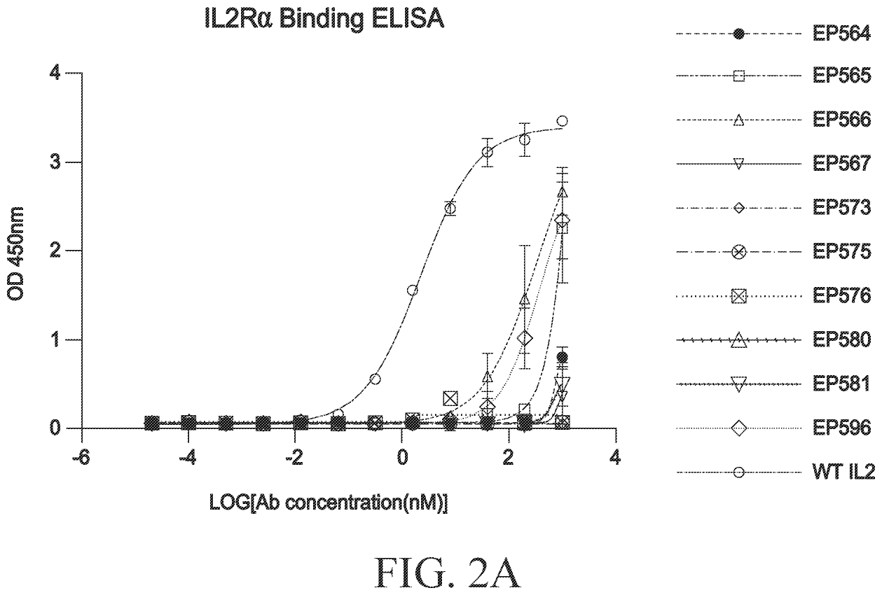

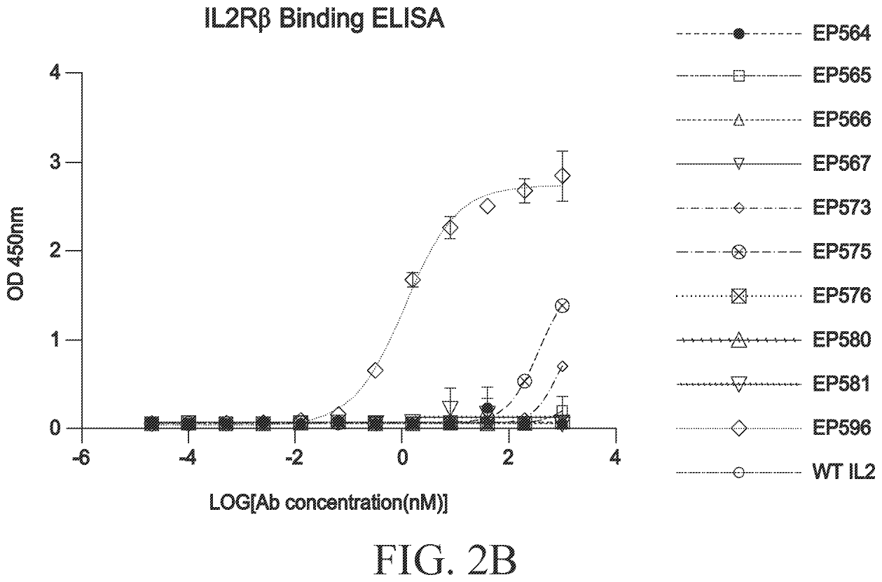

Mammalian cell-produced agonists were characterized for their binding activities to respective IL2Rβ and IL2Rα receptors by ELISA. Briefly, 384 well plate was immobilized with human IL2Rα and IL2Rβ Fc fusion proteins at a final concentration of 2 μg/mL in 1× PBS in total volume of 25 μL per well. The plate was incubated overnight at 4° C. and blocked with 80 μL of superblock per well for 1 hour. The purified clones were added to IL2Rα or IL-2Rβ wells in parallel in duplicates as a titration, with the highest concentration of 1 μM, followed by a 10 point 5× serial dilution of the clones. The binding activity was detected by adding 25 μL of anti-His HRP diluted at 1:5000 in 1× PBST. In between each step, the plate was washed three times with 1× PBST using a plate washer. The plate was then developed with 25 μL of TMB substrate for five minutes and stopped by adding 25 μL of 2N sulfuric acid. The plate was read at OD450 nm Biotek plate reader and the bar graph at 1 μM of IL2 was generated with Prism 8.1 software (FIG. 2).

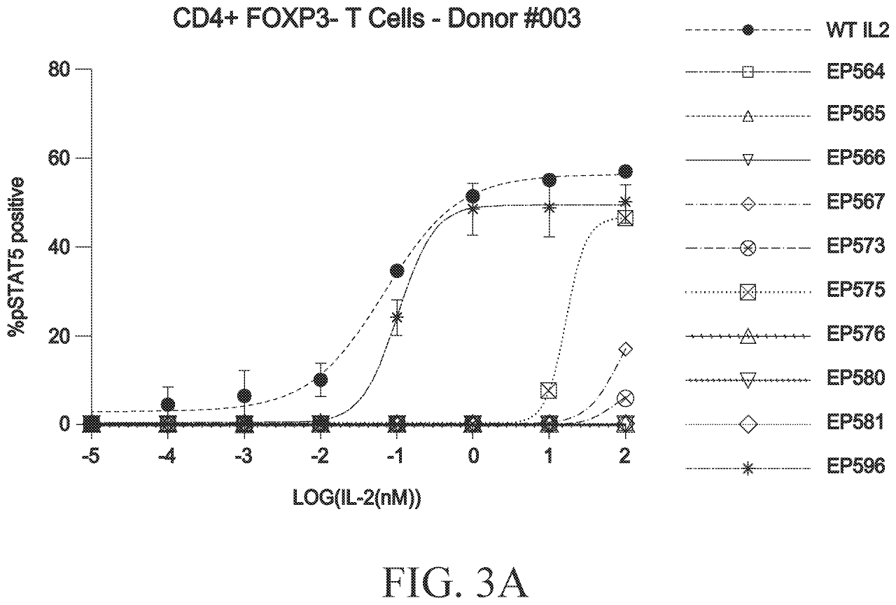

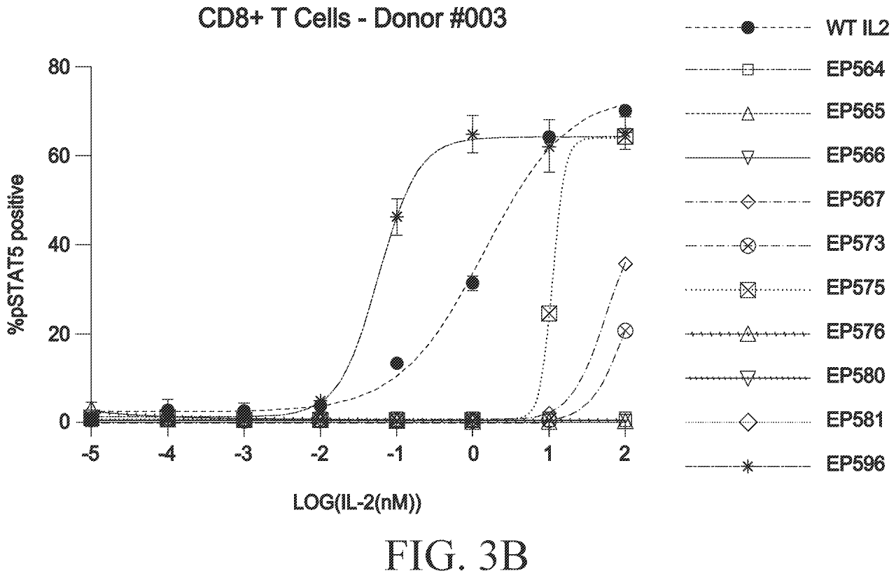

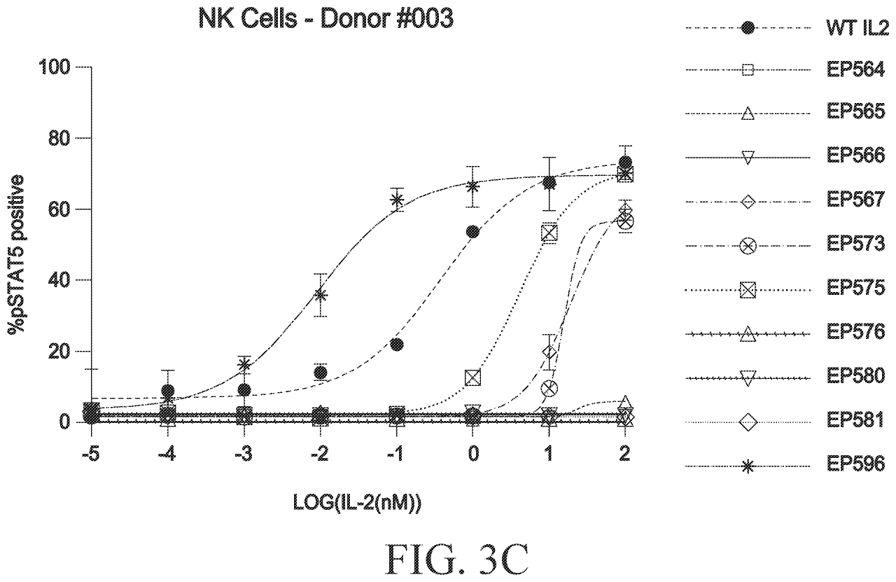

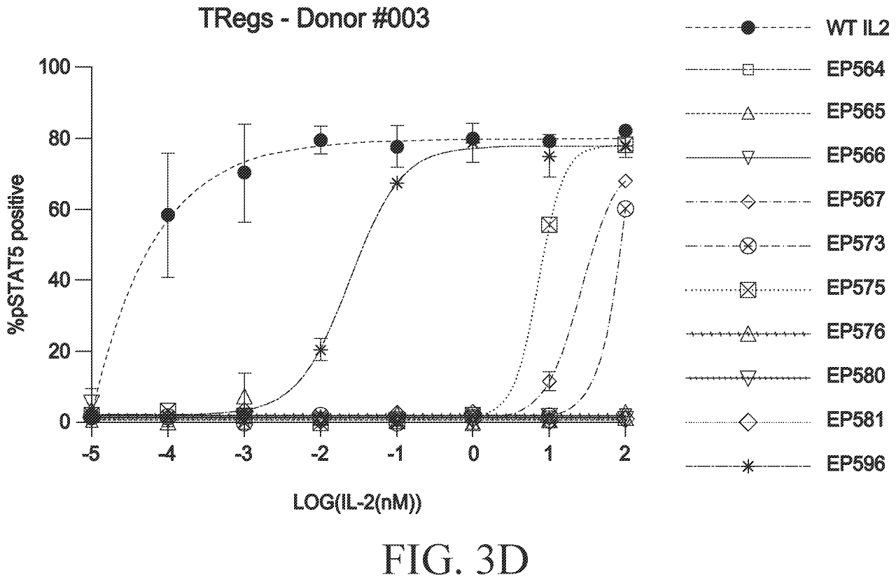

To further assess the immune cell modulation activities of the purified IL2 clones, Human PBMCs were isolated from peripheral blood of three separate donors, stained with viability dye, and plated at 250,000 cells/well in a 96-well plate in 90 μL of media. Cells were rested 1 hr at 37° C. Cells were stimulated with human IL2 WT, IL2Rβ binding enhanced EP596, and engineered His-Flag tagged IL2 at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIG. 3). P-STAT5 activation were determined using Prism software and summarized in Table 4.

TABLE 4
Summary of IL2R binding activities of mammalian-produced IL2Rβ agonist clones.

| | | | | p-STAT5 (EC50 nM) | | | |
|---|---|---|---|---|---|---|---|
| | | Receptor ELISA | | CD4+ | | | |
| | | Binding | | FOXP3-T | CD8+ T | | |
| Construct | Clones | IL2Rα | IL2Rβ | Cells | Cells | NK Cells | TRegs |
| WT IL2 | | Strong | Medium | 0.06801 | 1.371 | 0.3834 | <0.001 |
| EP596 | | Weak | Strong | 0.1036 | 0.05729 | 0.008875 | 0.02417 |
| EP564 | 2021EP22- | No | Very Weak | ND | ND | ND | ND |
| | E11 | | | | | | |
| EP565 | 2021EP22- | Medium | Medium | ND | ND | ND | ND |
| | F10 | | | | | | |
| EP566 | 2021EP22- | No | Very weak | ND | ND | ND | ND |
| | H09 | | | | | | |
| EP567 | 2021EP21- | Weak | Weak | Very Weak | Very Weak | Very Weak | Very |
| | C04 | | | | | | Weak |
| EP568 | 2021EP16- | NA | NA | N/A | N/A | N/A | N/A |
| | H06 | | | | | | |
| EP569 | 2021EP22- | NA | NA | N/A | N/A | N/A | N/A |
| | E03 | | | | | | |
| EP570 | 2021EP21- | NA | NA | N/A | N/A | N/A | N/A |
| | E03 | | | | | | |
| EP571 | 2021EP22- | NA | NA | N/A | N/A | N/A | N/A |
| | H02 | | | | | | |
| EP572 | 2021EP21- | NA | NA | N/A | N/A | N/A | N/A |
| | C03 | | | | | | |
| EP573 | 2021EP16- | No | Weak | Very Weak | Very Weak | Very Weak | Very |
| | E02 | | | | | | Weak |
| EP574 | 2021EP22- | NA | NA | N/A | N/A | N/A | N/A |
| | B04 | | | | | | |
| EP575 | 2021EP16- | No | Weak | Weak | Weak | Weak | Weak |
| | D09 | | | | | | |
| EP576 | 2021EP21- | No | No | ND | ND | ND | ND |
| | C10 | | | | | | |
| EP577 | 2021EP16- | NA | NA | N/A | N/A | N/A | N/A |
| | E05 | | | | | | |
| EP578 | 2021EP16- | NA | NA | N/A | N/A | N/A | N/A |
| | H02 | | | | | | |
| EP579 | 2021EP16- | NA | NA | N/A | N/A | N/A | N/A |
| | E11 | | | | | | |
| EP580 | 2021EP16- | No | NA | ND | ND | ND | ND |
| | H09 | | | | | | |
| EP581 | 2021EP16- | Weak | Weak | ND | ND | ND | ND |
| | C10 | | | | | | |
| EP582 | 2021EP16- | NA | NA | N/A | N/A | N/A | N/A |
| | G10 | | | | | | |
| EP583 | 2021EP16- | NA | NA | N/A | N/A | N/A | N/A |
| | H10 | | | | | | |
| EP584 | 2021EP16- | NA | NA | N/A | N/A | N/A | N/A |
| | F05 | | | | | | |
| EP585 | 2021EP16- | NA | NA | N/A | N/A | N/A | N/A |
| | C03 | | | | | | |
| EP586 | 2021EP16- | NA | NA | N/A | N/A | N/A | N/A |
| | E07 | | | | | | |

WT IL2 and EP596 used as references in ELISA binding analysis. Strong: OD450 > 1.5; medium: 0.7 < OD450<1.5; weak: 0.3 < OD450 < 0.7; very weak: 0.1 < OD450 < 0.3; No binding: OD450 < 0.1. For p-STAT5 analysis, very weak activities were defined as above base line reading at highest concentration in the assay.
ND: not detectable; NA: not available.

### Example 4

### Rational Generation of IL2RB EP575 Agonist Back-Mutation Clones

Rationally designed IL2Rβ agonist back-mutation strategy was carried out to create a range of IL2Rβ agonist candidate mutations. EP575 contains P82N, R83K, D84G, 185I, 186L, S87G, N88D, N90S, V91N and E95A mutations. The 185 and L86 were back-mutated to 85L and 86I of WT IL2 while 87G, 91N and 95A mutations were kept for all mutations. A systemic back-mutation was then applied to the other 6 residues so that each back-mutated IL2 will eventually contain a total of 5 mutations including 87G, 91N and 95A sites. A total of 20 back-mutation combinations were designed and the mutations were created by site directed mutagenesis using EP575 as template (Table 5). IL2Rβ agonist back-mutation clones were sequence verified after mutagenesis.

TABLE 5
List of EP575 back mutations. Residues highlighted
in bold are mutations from the WT-IL2.

| Constructs | 82 | 83 | 84 | 85 | 86 | 87 | 88 | 89 | 90 | 91 | 92 | 93 | 94 | 95 |
|---|---|---|---|---|---|---|---|---|---|---|---|---|---|---|
| WT-IL2 | P | R | D | L | I | S | N | I | N | V | I | V | L | E |
| EP575 | N | K | G | I | L | G | D | I | S | N | I | V | L | A |
| EP658 | P | R | G | L | I | S | D | I | S | N | I | V | L | A |
| EP659 | P | R | G | L | I | G | N | I | S | N | I | V | L | A |
| EP660 | P | R | G | L | I | G | D | I | N | N | I | V | L | A |
| EP661 | P | K | G | L | I | S | N | I | S | N | I | V | L | A |
| EP662 | P | K | G | L | I | S | D | I | N | N | I | V | L | A |
| EP663 | P | K | G | L | I | G | N | I | N | N | I | V | L | A |
| EP664 | N | R | G | L | I | S | N | I | S | N | I | V | L | A |
| EP665 | N | R | G | L | I | S | D | I | N | N | I | V | L | A |
| EP666 | N | R | G | L | I | G | N | I | N | N | I | V | L | A |
| EP667 | N | K | G | L | I | S | N | I | N | N | I | V | L | A |
| EP668 | P | R | G | L | I | G | D | I | S | N | I | V | L | A |
| EP669 | P | K | G | L | I | S | D | I | S | N | I | V | L | A |
| EP670 | P | K | G | L | I | G | N | I | S | N | I | V | L | A |
| EP671 | P | K | G | L | I | G | D | I | N | N | I | V | L | A |
| EP672 | N | R | G | L | I | S | D | I | S | N | I | V | L | A |
| EP673 | N | R | G | L | I | G | N | I | S | N | I | V | L | A |
| EP674 | N | R | G | L | I | G | D | I | N | N | I | V | L | A |
| EP675 | N | K | G | L | I | S | N | I | S | N | I | V | L | A |
| EP676 | N | K | G | L | I | S | D | I | N | N | I | V | L | A |
| EP677 | N | K | G | L | I | G | N | I | N | N | I | V | L | A |

### Example 5

### Characterization of IL2RB Agonist-Back-Mutation Clones

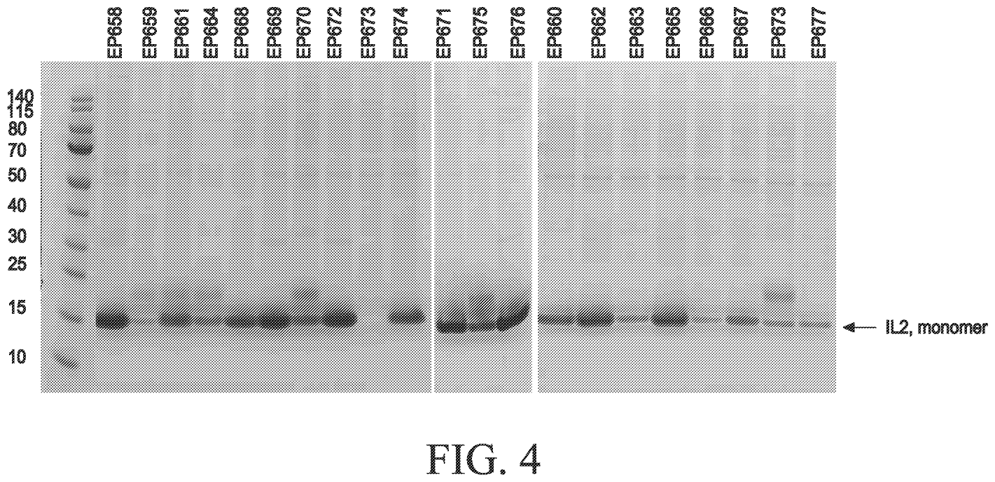

EP575 back-mutation clones were expressed in mammalian cells as described. Clones were purified by Ni Sepharose column and Superdex 200 Increase 10/300 GL column (GE Healthcare) to homogeneity. Clones were concentrated to appropriate concentration and stored in 1×PBS/300 nM NaCl buffer for binding, functional and mechanism analysis. The example of SDS-PAGE (sodium dodecyl sulfate-polyacrylamide gel electrophoresis) results of individual IL2 after Ni column purification was illustrated in FIG. 4.

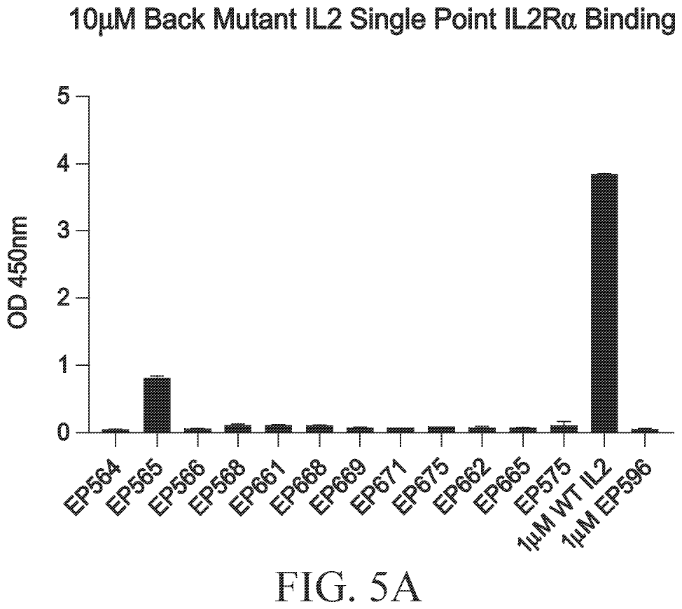

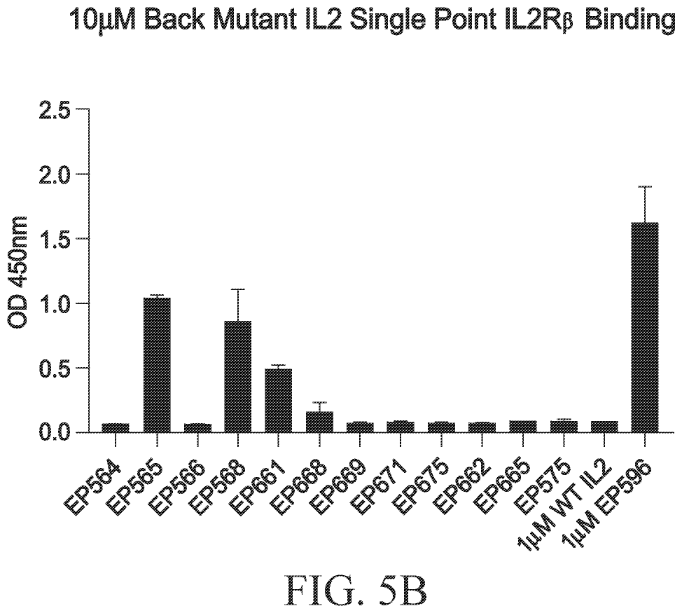

Back-mutations were characterized for their binding activities to IL2Rβ and IL2Rα receptors by ELISA. Briefly, 384 well plate was immobilized with human IL2Rα and IL2Rβ Fc fusion proteins at a final concentration of 2 μg/mL in 1× PBS in total volume of 25 μL per well. The plate was incubated overnight at 4° C. and blocked with 80 μL of superblock per well for 1 hour. Each of the purified EP575 back-mutation clones was added to IL2Rα or IL-2Rβ wells in parallel in duplicates to a final concentration of 10 μM. The IL2 mutant binding was detected by adding 25 μL of anti-His HRP diluted at 1:5000 in 1× PBST. In between each step, the plate was washed three times with 1× PBST using a plate washer. 1 μM WT IL2 and 1 μM EP596 were used as reference IL2s. The plate was then developed with 25 μL of TMB substrate for five minutes and stopped by adding 25 μl of 2N sulfuric acid. The plate was read at OD450 nm Biotek plate reader (Table 6) and the data was analyzed with Prism 8.1 software to generate bar graph (FIG. 5).

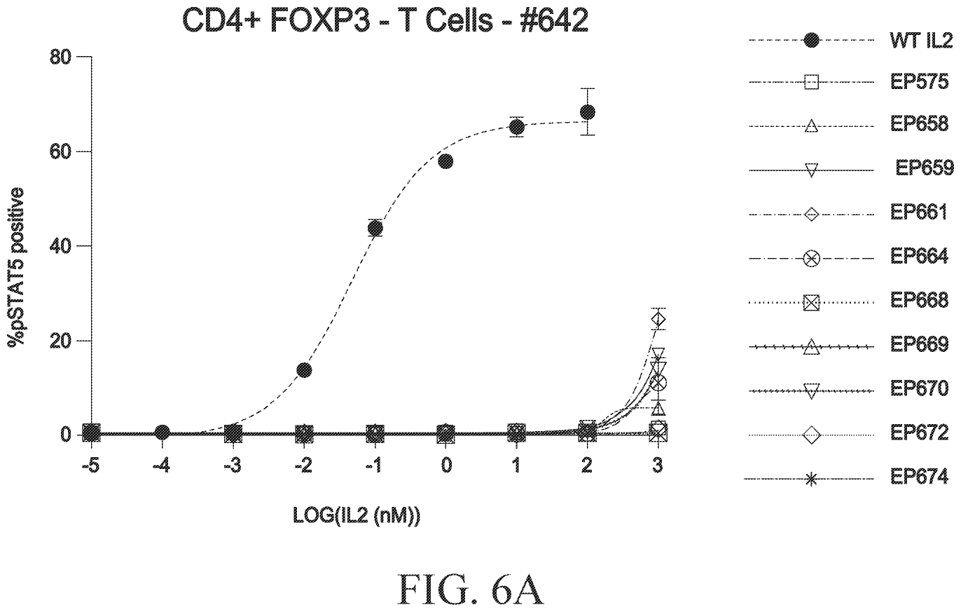

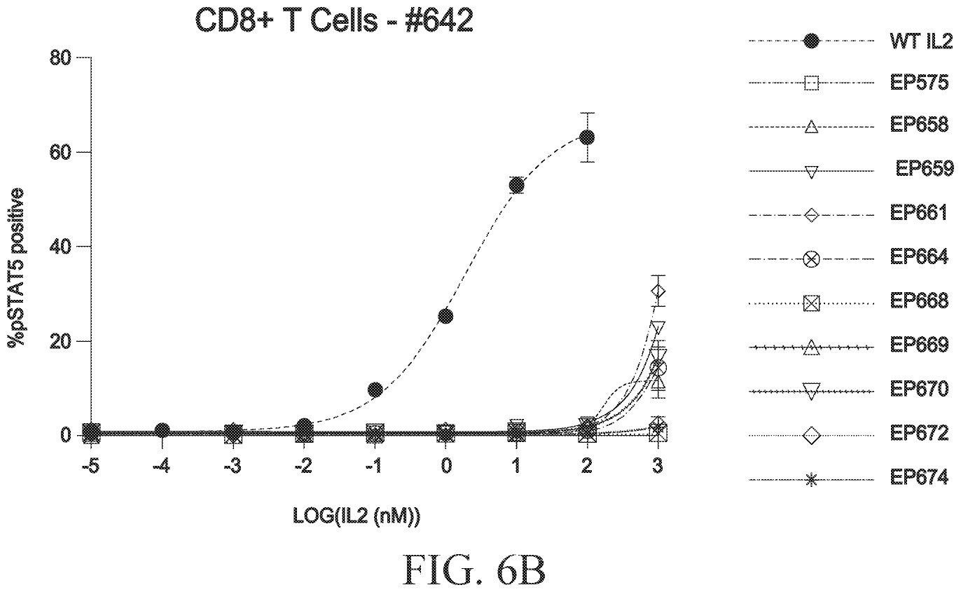

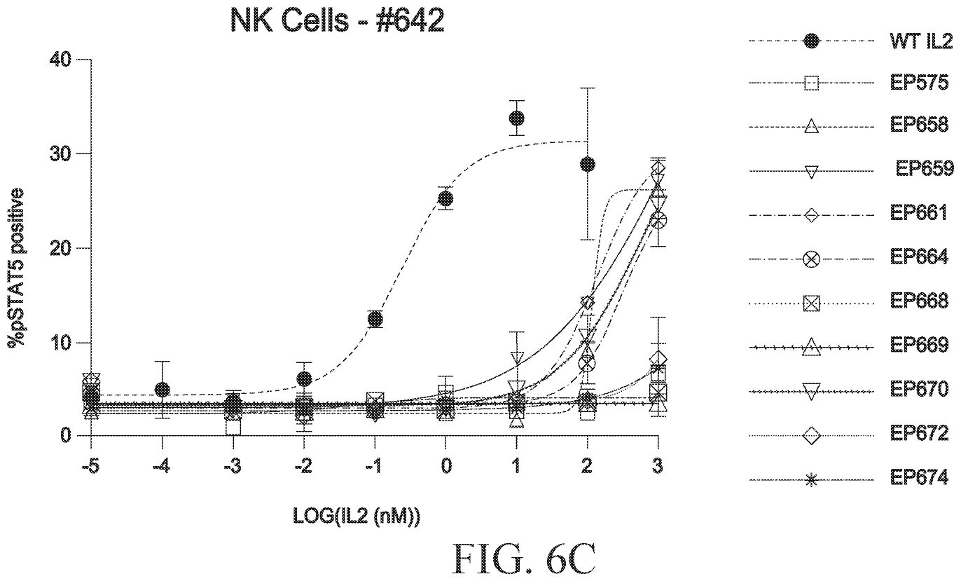

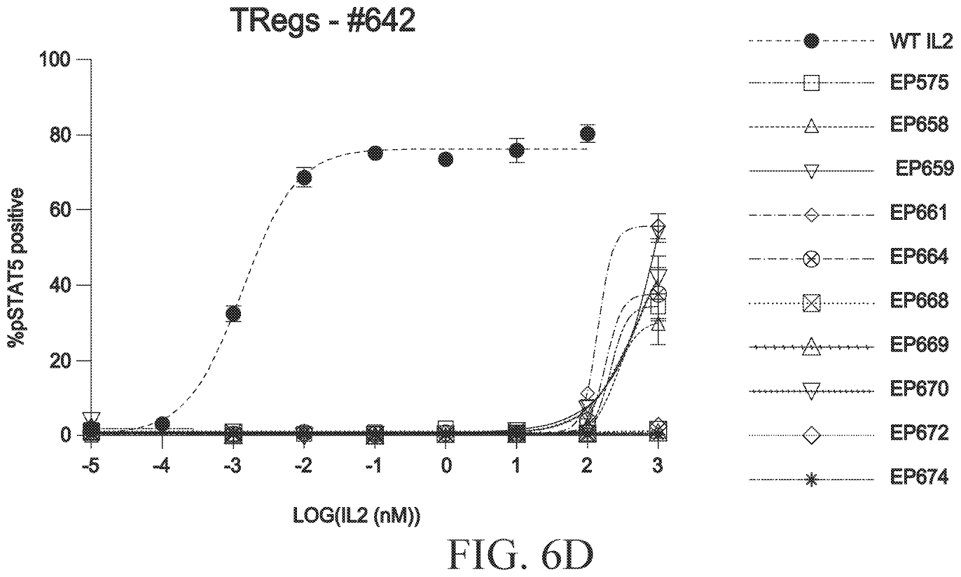

To further assess the immune cell modulation activities of the purified IL2 back-mutation clones, Human PBMCs were isolated from peripheral blood of three separate donors, stained with viability dye, and plated at 250,000 cells/well in a 96-well plate in 90 μL of media. Cells were rested 1 hr at 37° C. Cells were stimulated with human IL2 wt and engineered His-tagged IL2 at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIG. 6). P-STAT5 activation were determined using Prism software and summarized in Table 6. Those data supported that the engineered IL2 showed reduced binding to IL2Rα and IL2Rβ receptors. Correspondingly, the engineered IL2s showed weak p-STAT5 phosphorylation activities with incomplete curve or low activities at high concentrations compared to that of WT IL2 (FIG. 6).

TABLE 6
Summary of IL2Rα and IL2Rβ binding and
p-STAT5 activities of EP575 back mutations.

| | ELISA Binding | | | | | |
|---|---|---|---|---|---|---|
| | Average OD (450 nm) | | | | | |
| | at SinglePoint | | p-STAT5 activities | | | |
| | concentration | | EC50 (nM) | | | |
| Constructs | IL2Rα | IL2Rβ | CD8+ | CD4+ | NK | Treg |
| WT IL2 | 3.85 | 0.093 | 1.89 | 0.049 | 0.25 | 0.0013 |
| EP596 | 0.052 | 1.63 | NA | NA | NA | NA |
| EP575 | 0.113 | 0.093 | ND | ND | ND | ND |
| EP658 | 0.12 | 0.87 | ND | ND | ND | ND |
| EP659 | NA | NA | NA | NA | NA | NA |
| EP660 | NA | NA | NA | NA | NA | NA |
| EP661 | 0.11 | 0.501 | ND | ND | ND | ND |
| EP662 | 0.083 | 0.083 | ND | ND | ND | ND |
| EP663 | NA | NA | NA | NA | NA | NA |
| EP664 | NA | NA | NA | NA | NA | NA |
| EP665 | 0.077 | 0.096 | ND | ND | ND | ND |
| EP666 | NA | NA | NA | NA | NA | NA |
| EP667 | NA | NA | NA | NA | NA | NA |
| EP668 | 0.113 | 0.17 | ND | ND | ND | ND |
| EP669 | 0.082 | 0.082 | ND | ND | ND | ND |
| EP670 | NA | NA | NA | NA | NA | NA |
| EP671 | 0.082 | 0.09 | ND | ND | ND | ND |
| EP672 | NA | NA | NA | NA | NA | NA |
| EP673 | NA | NA | NA | NA | NA | NA |
| EP674 | NA | NA | NA | NA | NA | NA |
| EP675 | 0.084 | 0.082 | ND | ND | ND | ND |
| EP676 | NA | NA | NA | NA | NA | NA |
| EP677 | NA | NA | NA | NA | NA | NA |

ND: not detectable due to incomplete curve or very low activities.
NA: not available.

### Example 6

### Generation of IR2RB EP658 And EP661 Agonists Back-Mutations

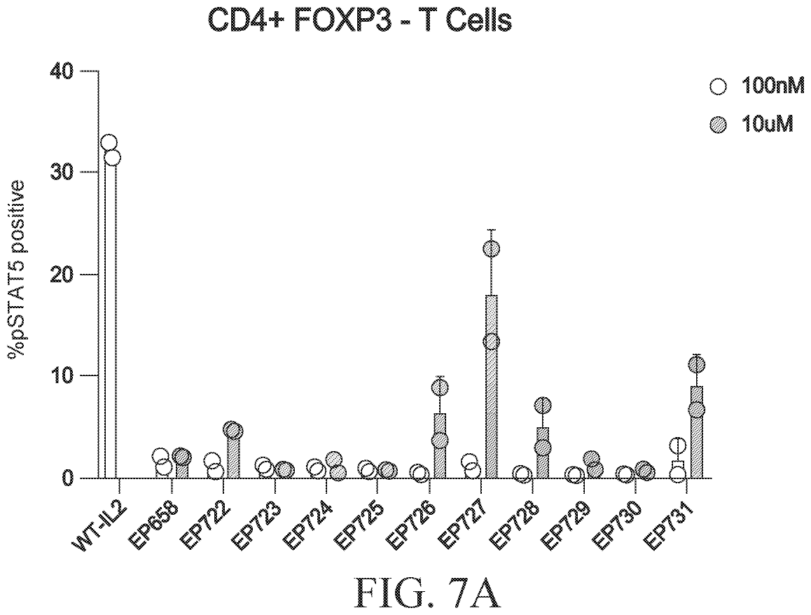

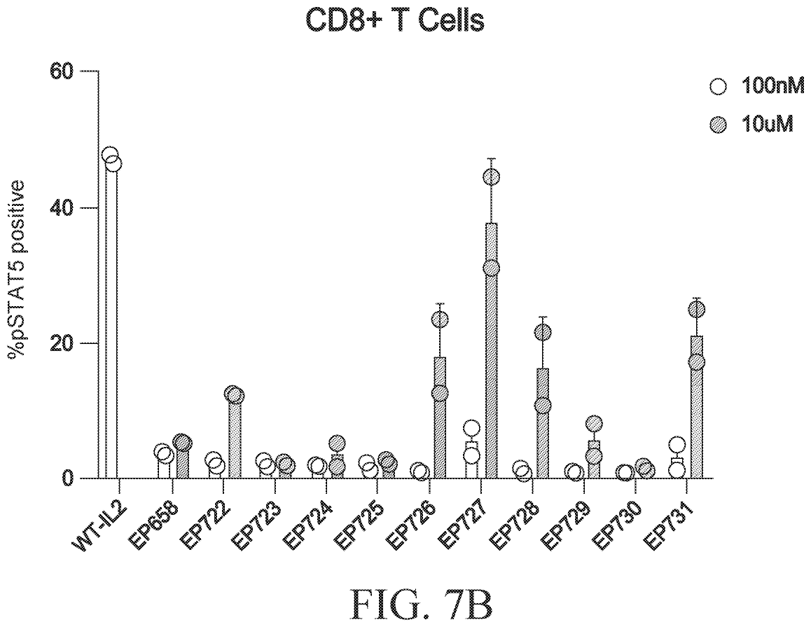

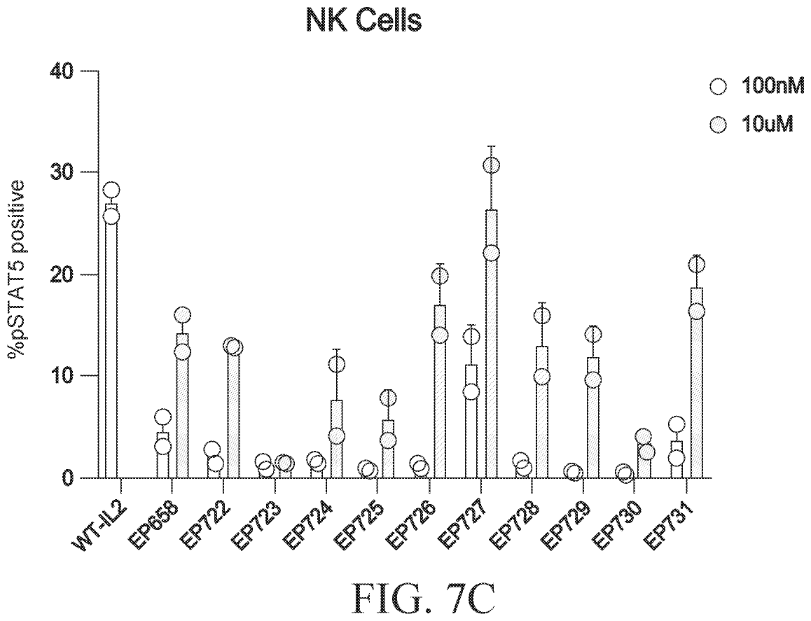

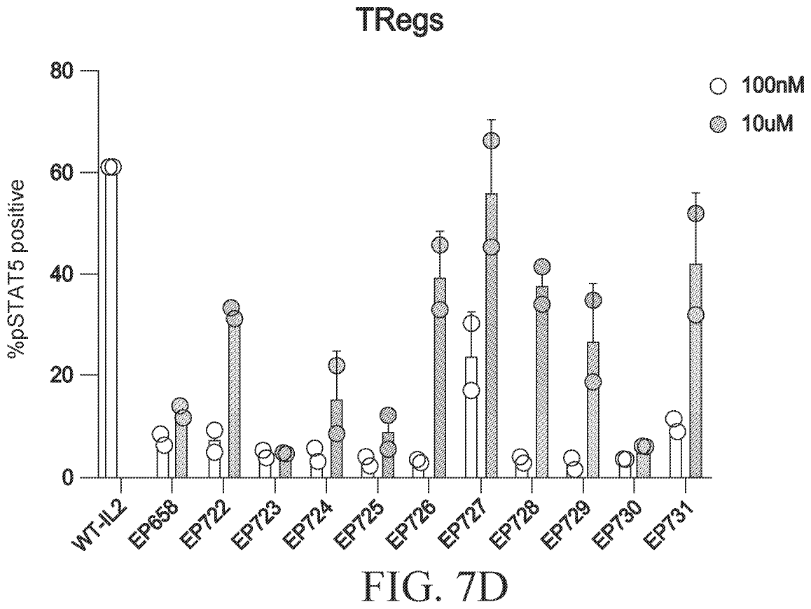

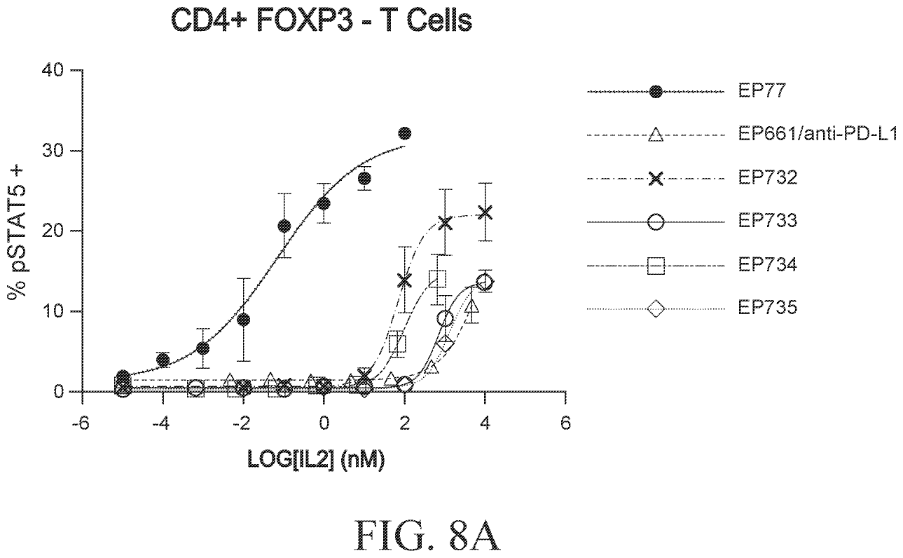

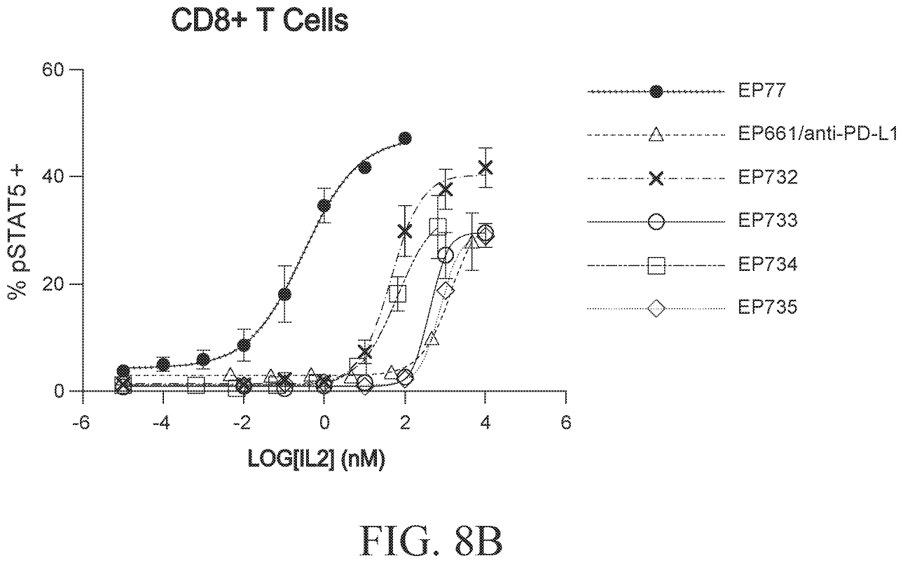

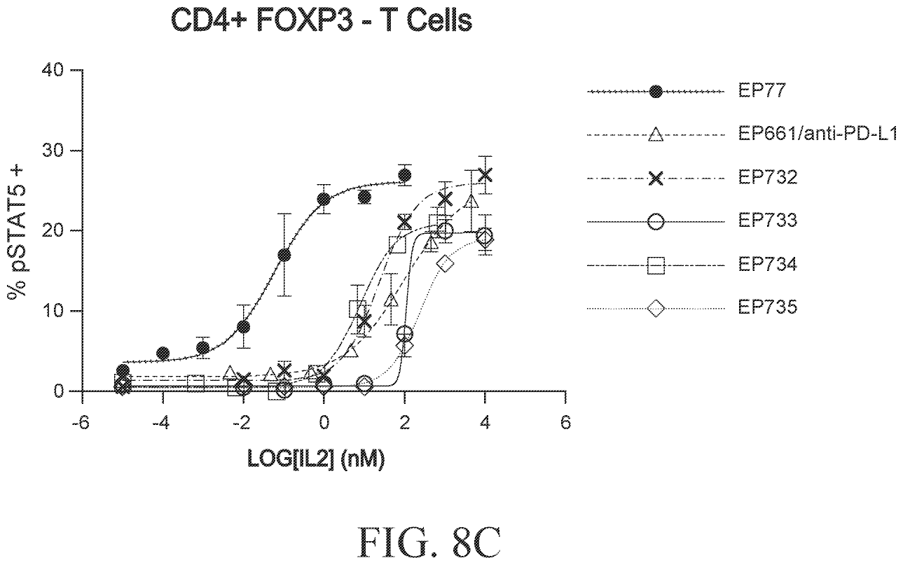

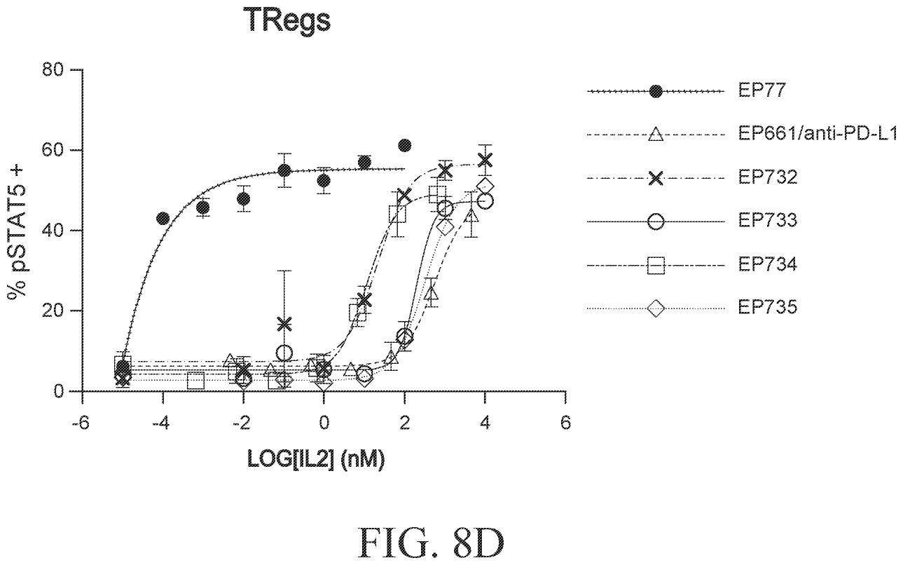

EP658 and EP661 were taken as examples for another round of engineering to further reduce the total numbers of mutation sites in engineered IL2s. EP658 contains D84G, N88D, N90S, V91N and E95A mutations, while EP661 contains R83K, D84G, N90S, V91N and E95A mutations. EP658 was applied for single point and two sites back mutation to generate 4 and 3 mutation sites containing IL2 mutants. Whereas single point mutagenesis was applied to EP661 to generate 4-point mutation containing IL2 mutants. A total of 14 back-mutation combinations were designed and the mutations were created by site directed mutagenesis using EP658 and EP661 as template, respectively (Table 7). Those back-mutation clones were sequence verified after mutagenesis and produced in mammalian cells as described above for further characterization. FIG. 7 and FIG. 8 show the immune cell p-STAT5 activities of EP658 and EP661 back mutations after treating healthy human PBMC donors, respectively.

TABLE 7
List of EP658 and EP661 back mutations. Residues highlighted
in bold are mutations from the WT-IL2.

| Constructs | 82 | 83 | 84 | 85 | 86 | 87 | 88 | 89 | 90 | 91 | 92 | 93 | 94 | 95 |
|---|---|---|---|---|---|---|---|---|---|---|---|---|---|---|
| WT-IL2 | P | R | D | L | I | S | N | I | N | V | I | V | L | E |
| EP575 | N | K | G | I | L | G | D | I | S | N | I | V | L | A |
| EP658 | P | R | G | L | I | S | D | I | S | N | I | V | L | A |
| EP661 | P | K | G | L | I | S | N | I | S | N | I | V | L | A |
| EP722 | P | R | D | L | I | S | D | I | S | N | I | V | L | A |
| EP723 | P | R | G | L | I | S | D | I | N | N | I | V | L | A |
| EP724 | P | R | G | L | I | S | D | I | S | V | I | V | L | A |
| EP725 | P | R | G | L | I | S | D | I | S | N | I | V | L | E |
| EP726 | P | R | D | L | I | S | D | I | N | N | I | V | L | A |
| EP727 | P | R | D | L | I | S | D | I | S | V | I | V | L | A |
| EP728 | P | R | D | L | I | S | D | I | S | N | I | V | L | E |
| EP729 | P | R | G | L | I | S | D | I | N | V | I | V | L | A |
| EP730 | P | R | G | L | I | S | D | I | N | N | I | V | L | E |
| EP731 | P | R | G | L | I | S | D | I | S | V | I | V | L | E |
| EP732 | P | K | D | L | I | S | N | I | S | N | I | V | L | A |
| EP733 | P | K | G | L | I | S | N | I | N | N | I | V | L | A |
| EP734 | P | K | G | L | I | S | N | I | S | V | I | V | L | A |
| EP735 | P | K | G | L | I | S | N | I | S | N | I | V | L | E |

### Example 7

### Binding of IR2RB Reduced-Binding Agonists to Cell Surface Receptors

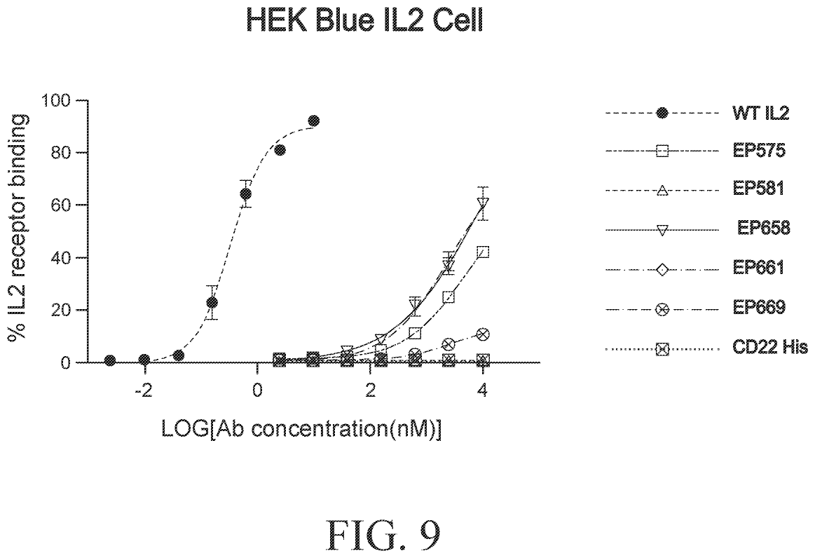

HEK Blue IL2 cells, expressing IL2 receptors alpha, beta, and gamma, were seeded at a density of 100,000 cells per well in a 96 well round bottomed plated in a total volume of 100 μL of media. Cells were rested in an incubator for 1 hour at 37° C. After 1 hour, the media was replaced with 100 μL of media containing a 7-point 4× titration of the IL2 muteins starting at 10 μM, and the plate was incubated, shaking, at 4° C. for 30 minutes. After washing off unbound IL2, the cells were resuspended in 100 μL of media containing 1:100 anti His Dylight 650 secondary antibody, and incubated shaking, at 4° C. for 30 minutes protected from light. Unbound secondary antibody was washed off, and the cells were resuspended in PBS containing 1:800 Zombie Aqua viability dye and incubated at room temperature for 5 minutes. Cells were washed twice, before being analyzed using an Attune NXT flow cytometer. The percentage of HEK Blue IL2 cells which were positive for IL2 mutein binding was determined (FIG. 9). The recombinant human CD22 was used as a negative control and the WT IL2 as the reference. Consistent with the binding and p-STAT5 data, the agonists showed partial binding to the cell surface IL2 receptors compared to a complete binding of WT-IL2.

### Example 8

### Design of IR2RB Agonist Fc-Fusion Proteins and Anti-PD-(L)1 Bispecific Antibodies

The engineered IL2Rβ reduced-binding agonist can be further developed into Fc fusion proteins in order to further improve its certain properties. To generate monovalent IL2-Fc fusion protein, the protein sequences encoding engineered IL2 polypeptides of EP567 (SEQ ID NO:49), EP575 (SEQ ID NO:57), EP581 (SEQ ID NO:63), EP658 (SEQ ID NO:69), EP660 (SEQ ID NO:71), EP661 (SEQ ID NO:72), EP669 (SEQ ID NO:80), and EP676 (SEQ ID NO: 87) were fused to the N-terminal site of the constant frame sequences of human IgG1 isoform, to produce engineered agonist-Fc fusion proteins (SEQ ID NO:137). The knob mutations of S354C, T366W and K409A were introduced to the constructs. The hole mutations of Y349C, T366S, L368A, F405K, Y407V were introduced to CH2 and CH3 fragments of IgG1. The L234A, L235A and P329G mutations in the human IgG1 were introduced to eliminate complement binding and Fc-γ dependent antibody-dependent cell-mediated cytotoxicity (ADCC) effects (Lo et al., JBC 2017). The DNA encoding the entire Fc fusion agonist protein was then synthesized with codon optimized for mammalian cell expression, and subcloned to pCDNA3.4 (Invitrogen).

On the other side, the engineered IL2Rβ reduced-binding agonist can be developed into bifunctional or bispecific antibodies in order to synergize its immune modulating activities with other therapeutic mechanisms. To generate anti-PD-L1-IL2 fusion bispecific antibody, the S354C, T366W and K409A mutations (Wei et al., Oncotarget, 2017; Xu et al., mAbs, 2015) were introduced to one heavy chain of the anti-PD-L1 antibody as a knob molecule (EP362; SEQ ID NO:104). The C-terminus of another heavy chain of the anti-PD-L1 was fused to human IL2 protein with a (G4S)4 linker. The S354C, Y349C, T366S, L368A, F405K, Y407V mutations were introduced to the make the chain as a hole molecule (e.g., EP643, in which the EP575 of engineered IL2 was fused). The L234A, L235A and P329G mutations in the human IgG1 were introduced to eliminate complement binding and Fc-γ dependent antibody-dependent cell-mediated cytotoxicity (ADCC) effects (Lo et al., JBC 2017).

In another example, the S354C, Y349C, T366S, L368A, F405K, Y407V mutations were introduced to the heavy chain of anti-PD-L1 sequence to make the chain as a hole molecule (EP325; SEQ ID NO:114). The knob molecule with N-terminal IL2 fusion (e.g., EP826) can form a monovalent (in Fab format) anti-PD-L1-IL2 bispecific antibody with that of EP325 and EP205, the light chain of the anti-PD-L1 antibody.

In another example, the variable heavy chain region of an anti-mouse PD-L1 or anti-mouse PD-1 antibody was fused to the constant region of human IgG1 antibody containing to generate chimeric anti-mouse PD1 or anti-mouse PD-L1 antibody, respectively. Knob and hole mutations along with ADCC silent mutations were introduced to the heavy chain. An engineered IL2 molecule was fused to the terminus of one of the heavy chain arms, so that the anti-mouse PD-1 or PD-L1 antibody binding sites may form a bispecific protein. Such bispecific proteins can serve as surrogate anti-PD-(L)1/IL2 bispecifics (e.g., bifunctional fusion protein complex) for investigating the mechanism of action and efficacy in mouse models.

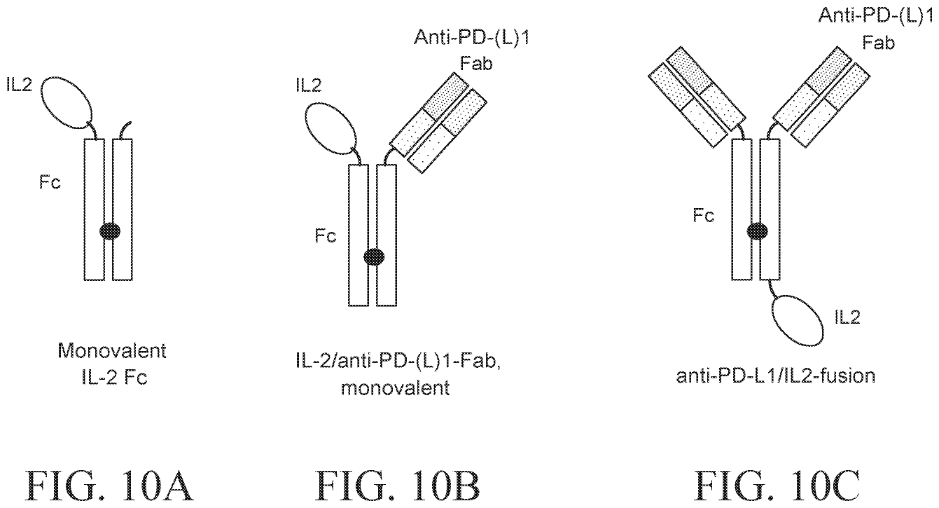

The DNA encoding the entire above designed sequences were then synthesized with codon optimized for mammalian cell expression, and subcloned to pCDNA3.4 (Invitrogen). FIG. 10 showed examples of schematic diagrams of the formats for selected anti-PD-L1 or anti-PD-1 antibodies.

### Example 9

### IL2-Fc Protein and Bispecific Antibodies Production

For monovalent IL2-Fc fusion protein production, the “knob” and “hole” constructs in respective IgG1 backbone format were transfected to ExpiHEK293-F cells with the ratio of 1:1. The cells were grown for five days and the supernatant was collected by centrifugation and filtered through a 0.2 μm PES membrane. The Fc fusion agonist first was purified by MabSelect PrismA protein A resin (GE Health). The protein was eluted with 100 mM Gly pH 2.5 and 150 mM NaCl and quickly neutralized with 20 mM citrate pH 5.0 and 300 mM NaCl. The agonist protein was then concentrated to 1 mL and further purified by a Superdex 200 16/600 gel filtration column. The monomeric peak fractions were pooled and concentrated. The final purified protein has endotoxin of lower than 10 EU/mg and kept in 1×PBS. The purified monovalent IL2-Fc fusion agonists were run on an SDS gel (4-12% Bis-Tris Bolt gel, with MES running buffer).

For the monovalent (in Fab format) anti-PD-L1-IL2 bispecific antibody production (e.g., bifunctional fusion protein), the “knob” and “hole” constructs in respective IgG1 backbone formats were transfected to ExpiHEK293-F cells in free style system (Invitrogen) according to standard protocol with ratio of knob:hole:light chain of 1:4:4. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5 and 150 mM NaCl and quickly neutralized with mM histidine pH 5.0+150 mM NaCl. The antibody was then further purified by a Superdex 200 16/600 column. The monomeric peak fractions were pooled and concentrated. The final purified protein has endotoxin of lower than 10 EU/mg and kept in 20 mN histidine, 150 mM NaCl buffer.

For the bivalent anti-PD-L1-IL2 and/or anti-PD-1-IL2 bispecific antibody production (e.g., bifunctional fusion protein complex), the “knob” and “hole” constructs in respective IgG1 backbone formats were transfected to ExpiHEK293-F cells in free style system (Invitrogen) according to standard protocol with ratio of knob:hole:light chain of 1:2:2. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5+150 mM NaCl and quickly neutralized with 20 mM histidine pH 5.0+150 mM NaCl. The antibody was then further purified by a Superdex 200 16/600 column. The monomeric peak fractions were pooled and concentrated. The final purified protein has endotoxin of lower than 10 EU/mg and kept in 20 mN histidine, 150 mM NaCl buffer.

The anti-PD-L1 monoclonal antibody was expressed transiently in ExpiHEK293-F cells in free style system (Invitrogen) according to standard protocol with a ratio of the plasmid DNA of heavy chain and light chain of 1:2. The cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5 and 150 mM NaCl and quickly neutralized with 20 mM citrate pH 5.0 and 300 mM NaCl. The antibody was then further purified by a Superdex 200 16/600 column. The monomeric peak fractions were pooled and concentrated. The final purified protein has endotoxin of lower than 10 EU/mg and kept in 20 mM Histidine pH 6.0 and 150 mM NaCl.

### Example 10

### Characterization of IL2RB Agonist Fc-Fusion and Bispecific Antibodies

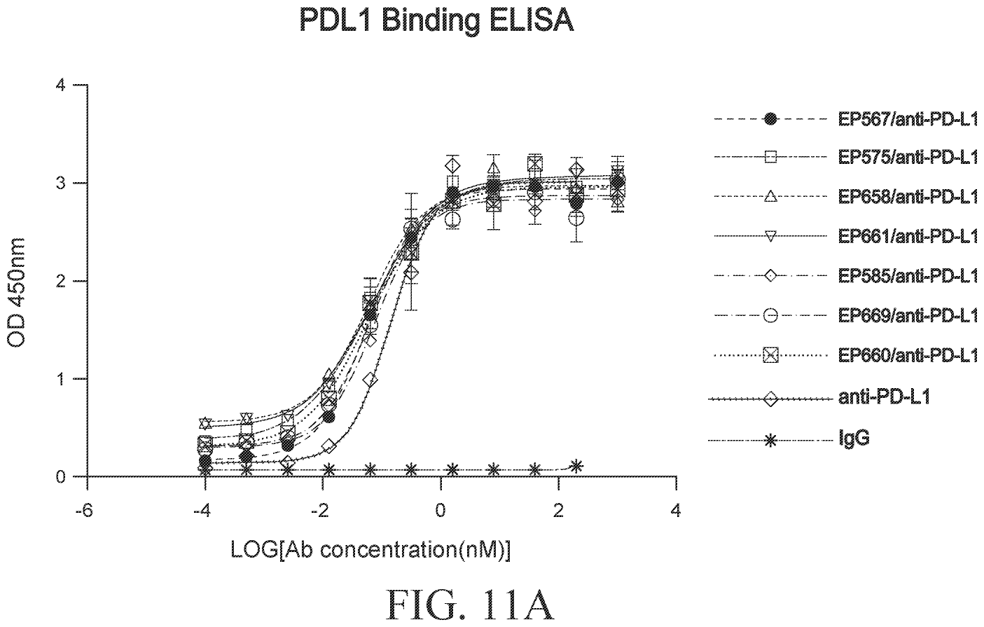

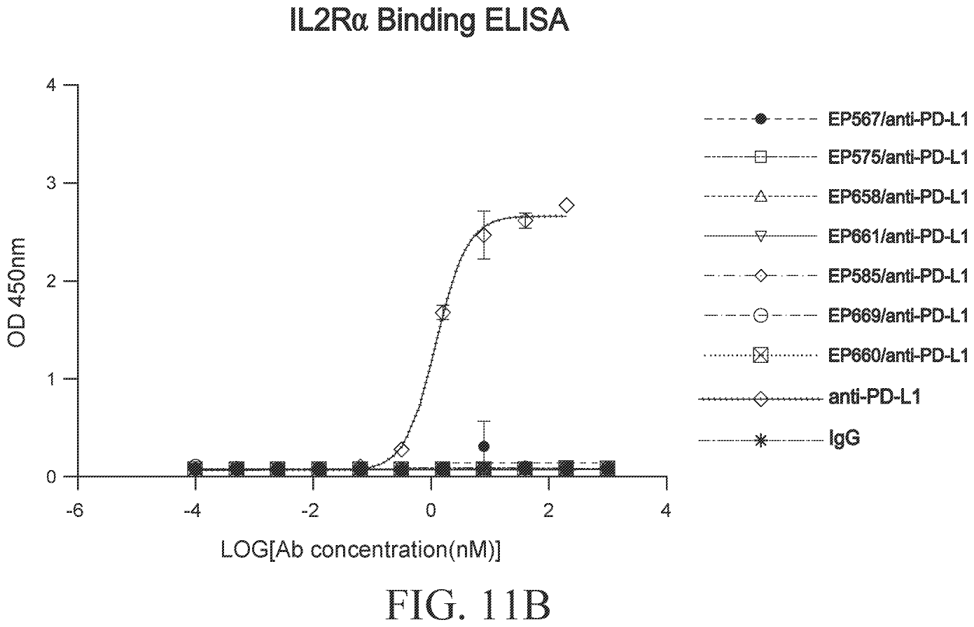

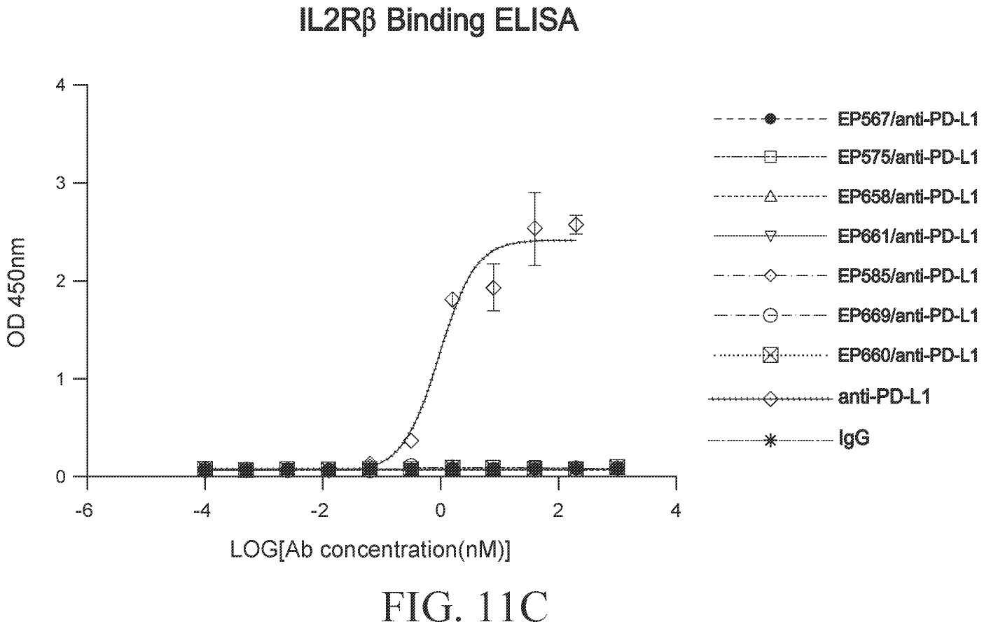

For anti-PD-L1/IL2 and/or anti-PD-1-IL2 bispecific antibodies, goat anti-human Fc was added in 25 μL of 1× PBS to wells of 384-well plate and incubated overnight at 4° C. to coat the plates. Plates were washed three times with 0.05% Tween20/1× PBS. Plates were blocked with 100 μL of SuperBlock for 1 hr at RT and then washed 3 times with 0.05% Tween20/1× PBS. Anti-PD-L1/IL2 mutant bispecific (e.g., bifunctional fusion protein complex) were diluted in 0.05% Tween 20/1× PBS from 1000 nM to 0 nM and added to plates for 2 hrs at room temperature. Plates were then washed 6 times with 0.05% Tween20/1× PBS. Captured protein was detected with biotinylated recombinant PD-(L)1, IL2Rα, or IL2Rβ, washed, and visualized with Streptavidin-HRP diluted 1:5000 in 0.05% Tween20/1× PBS. Plates were then washed 6 times with 0.05% Tween20/1× PBS, and TMB was added to develop blue color. Reactions were stopped with 2N hydrogen sulfide and light absorbance at 450 nm was read on a BioTek plate reader. Absorbance versus IL2 concentration is graphed for human PD-(L)1, IL2Rα, and IL2Rβ binding (FIG. 11). A summary of the PD-(L)1, IL2Rα and IL2Rβ ELISA binding activities is shown (Table 8). All the constructs showed similar PD-L1 binding activities. No observable IL2Rα and IL2Rβ binding activities at the highest concentration of 1 μM for EP567, EP575, EP581, EP658, EP660, EP661, and EP669 fused anti-PD-L1/IL2 bispecifics (e.g., bifunctional fusion protein complex), whereas the IL2Rβ agonist of EP415 fused bispecific showed strong IL2Rβ binding activity. The reference WT-IL2 fused bispecific showed strong IL2Rα binding which is consistent with reported data.

TABLE 8
Summary of PD-(L)1, IL2Rα and IL2Rβ binding
analysis of IL2 Fc fusion proteins

| | | ELISA EC50 (nM) | | |
|---|---|---|---|---|
| Constructs | Note | IL2Rα | Il2Rβ | PD-L1 |
| EP205/EP206 | Anti-PD-L1 | NA | NA | 0.14 |
| EP415/EP325/EP205 | anti-PD-L1-EP337-IL2 | ND | 0.95 | NA |
| EP362/EP363/EP205 | anti-PD-L1-WT-IL2 | 1.24 | ND | NA |
| EP362/EP642/EP205 | anti-PD-L1-EP567-IL2 | ND | ND | 0.059 |
| EP362/EP643/EP205 | anti-PD-L1-EP575-IL2 | ND | ND | 0.062 |
| EP362/EP736/EP205 | anti-PD-L1-EP658-IL2 | ND | ND | 0.062 |
| EP362/EP737/EP205 | anti-PD-L1-EP661-IL2 | ND | ND | 0.073 |
| EP362/EP741/EP205 | anti-PD-L1-EP581-IL2 | ND | ND | 0.074 |
| EP362/EP742/EP205 | anti-PD-L1-EP669-IL2 | ND | ND | 0.063 |
| EP362/EP743/EP205 | anti-PD-L1-EP660-IL2 | ND | ND | 0.062 |

### Example 11

### P-STAT5 Activation of Human PBMCS by IL2RB Agonist Fc-Fusion Bispecific Antibodies

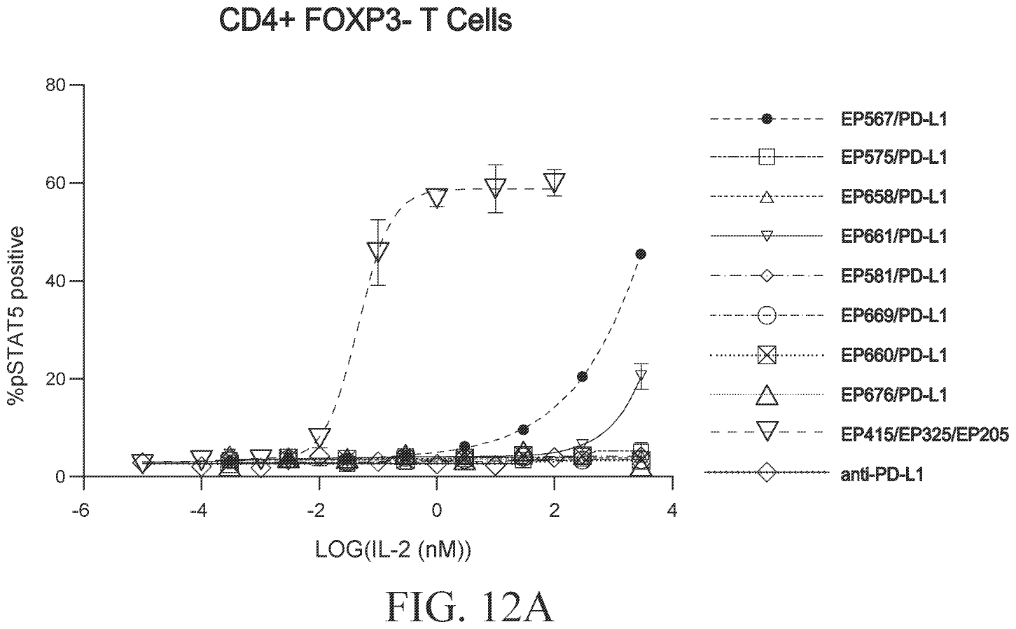

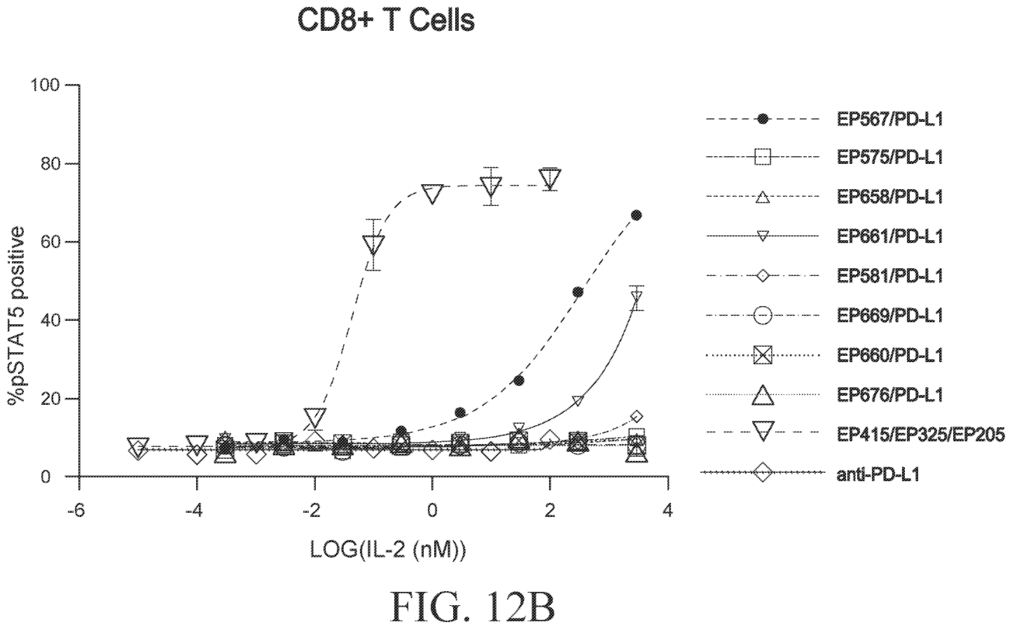

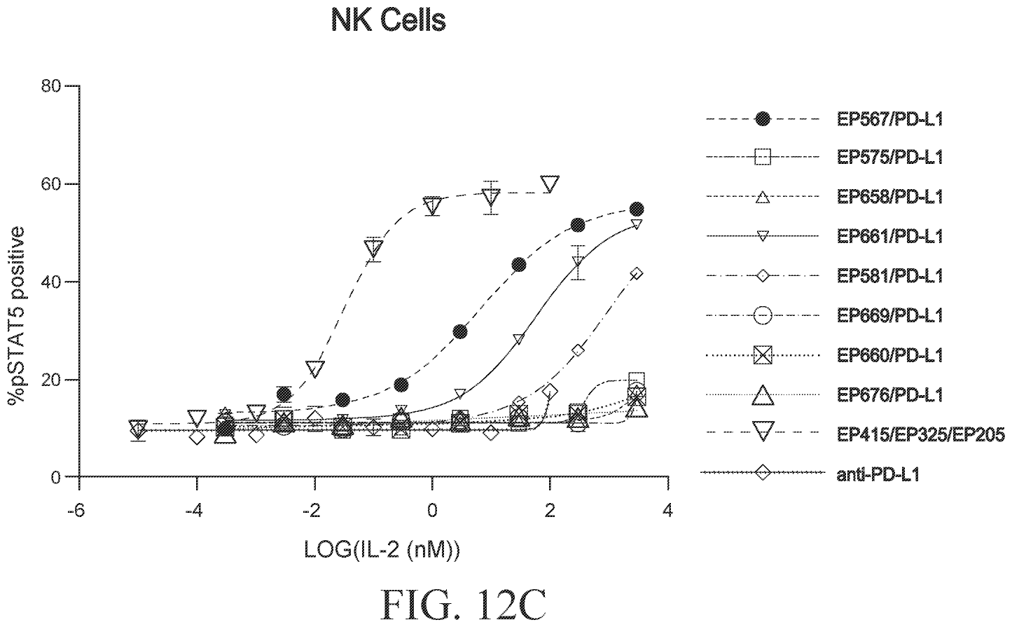

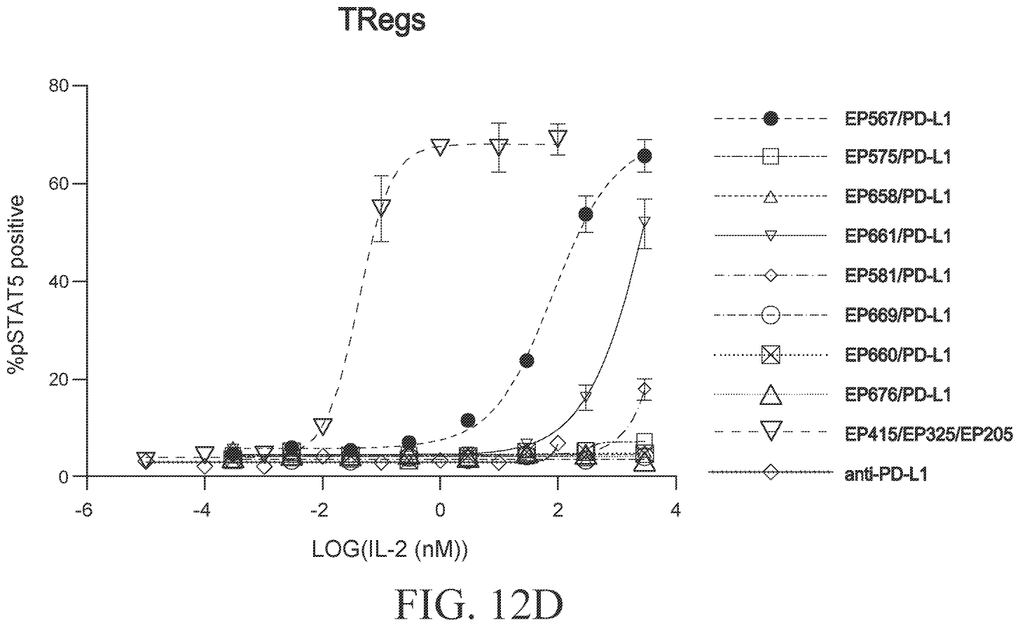

Human PBMCs were isolated from peripheral blood, stained with viability dye, and plated at 250,000 cells/well in a 96-well plate in 90 μl of media. Cells were rested 1 hr at 37° C. Cells were stimulated with human IL2 wt and IL2Rβ agonist Fc-fusion proteins at 10× concentration in 10 μl for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIG. 12).

### Example 12

### Safety and Immune Cell Activation of IL2RB Agonist EP567/Anti-PD-L1 in Mice

6-8-week-old female C57BL/6 mice were dosed on day 1 and day 4 with either vehicle, or EP567/PD-L1 at concentrations of 2.5 mg/kg, 5 mg/kg, or 10 mg/kg. Body weight was monitored daily and expressed as relative body weight, normalized to the pre-dose body weight. On Day 8, mice were sacrificed, and lung were harvested and measured.

During the day 8 sacrifice, blood was collected by cardiac puncture. After lysis of the red blood cells, the blood was stained for immune phenotype markers (CD3, CD4, CD8, NKp46, NK1.1, and FOXP3), as well as CD69, Ki67, and a viability marker. Cells were analyzed using an Attune NxT flow cytometer. The number of CD4+FOXP3− T cells, CD8+ T cells, NK cells, and Tregs was calculated as the percentage of live cells. The percentage of CD4+FOXP3− T cells, CD8+ T cells, and Tregs which were Ki67+ was calculated. The percentage of CD4+ FOXP3− T cells, CD8+ T cells, and Tregs which were CD69+ was calculated.

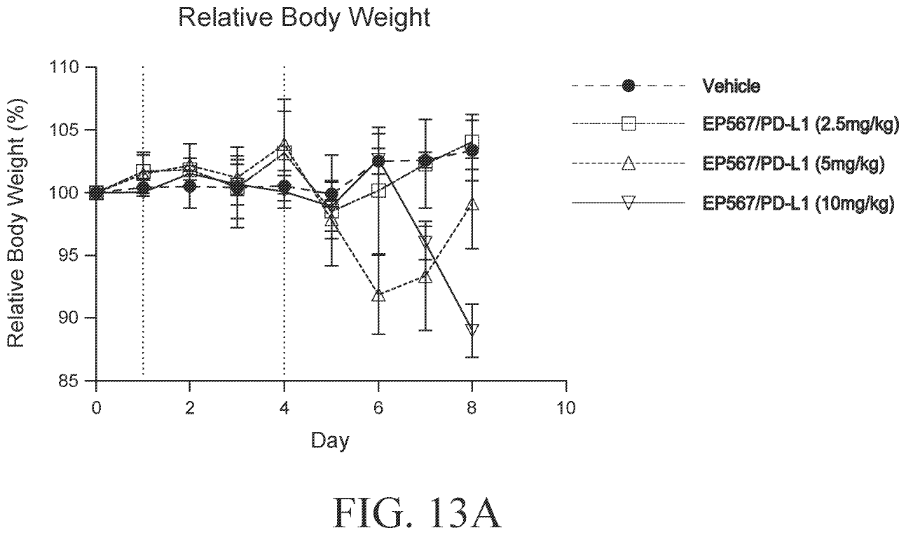

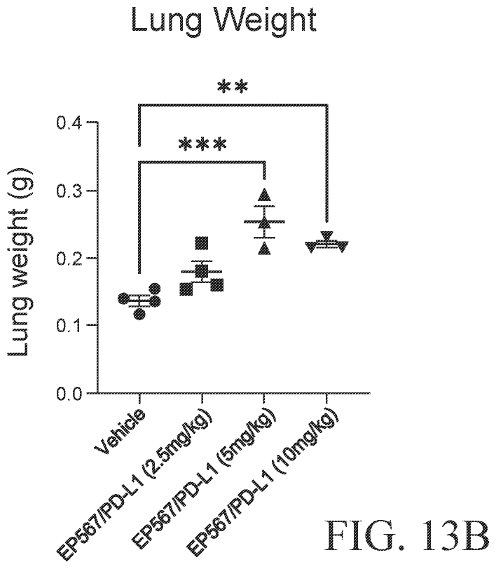

FIG. 13A shows the body weight change of the mice. EP567/anti-PD-L1 bispecific started to induce body weight loss at 5 mg/kg, and severe body weight loss and death at 10 mg/kg. accordingly, lung weight (FIG. 13B) increased proportionally with increased dose of EP567.

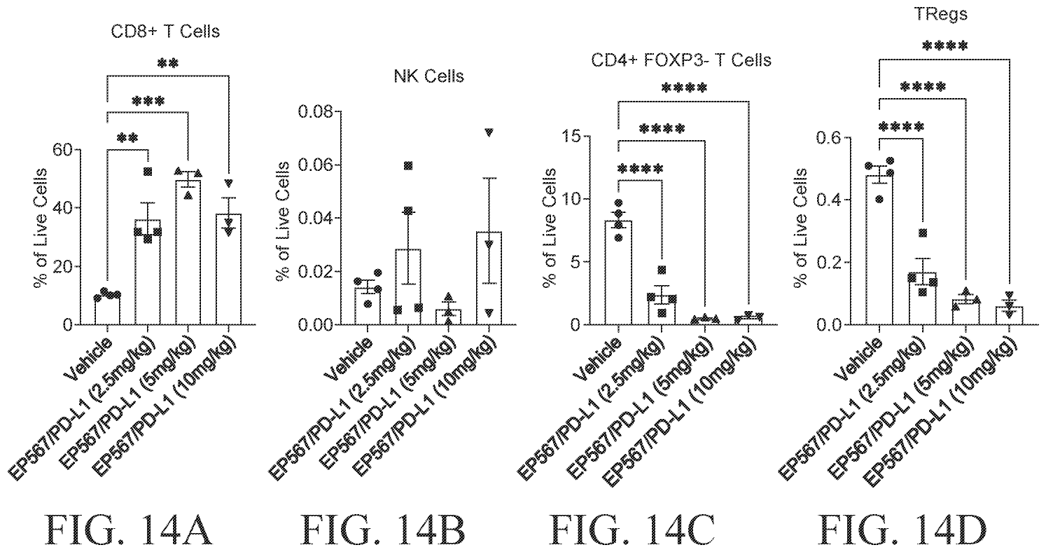

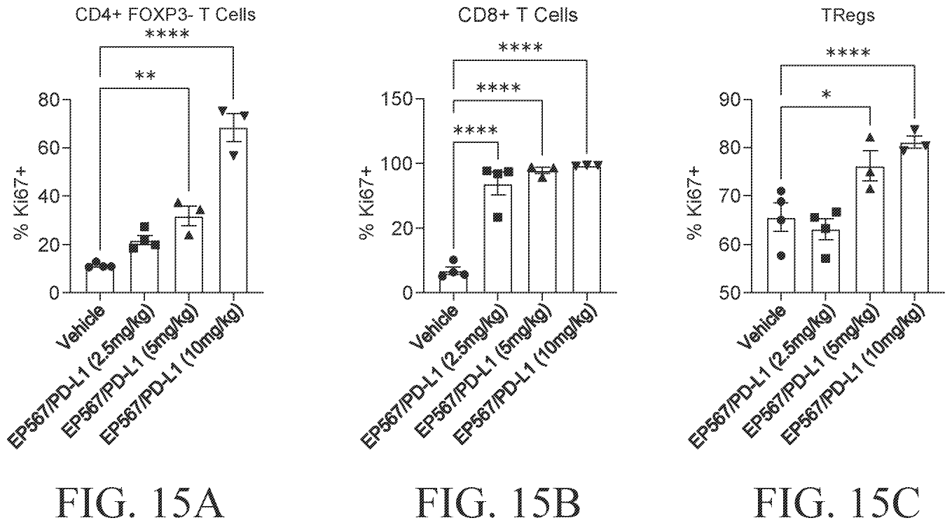

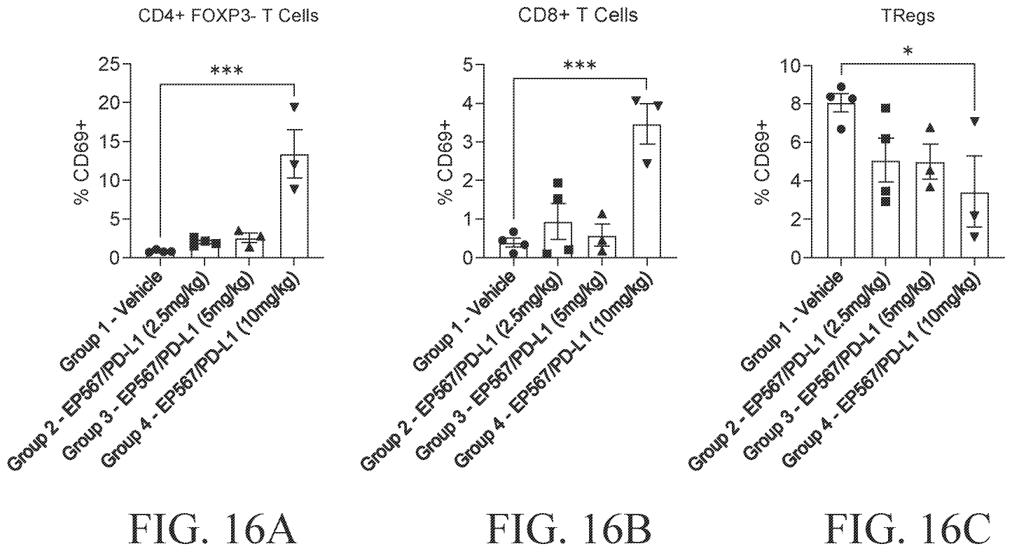

The peripheral immune cell profiling data supported that EP567/anti-PD-L1 increased the CD8 T cells levels and reduce the Treg cell levels in a dose dependent manner (FIG. 14). Correspondingly, EP567 increased the proliferation of CD8 T cells and downregulate the proliferation of Treg cells at all doses (FIG. 15). EP567 also induced early activation of CD8 and CD4 T cells at high dose of 10 mg/kg (FIG. 16). Together, the data supported EP567/anti-PD-L1 can stimulate immune cell activation, however, mice were not tolerating the high concentration of the bispecific, indicating more engineered IL2 should be evaluated.

### Example 13

### Safety and Immune Cell Activation of Additional IL2/ANTI-PD-L1 Bispecific in Mice

6-8 week old female C57BL/6 mice were dosed on day 0 and day 4 with 10 mg/kg of IL2/anti-PD-L1 bispecifics. Body weight was monitored 5 times per week and expressed as relative body weight, normalized to the pre-dose body weight. On Day 8, mice were sacrificed, and lung were harvested and measured.

During the day 8 sacrifice, blood was collected by cardiac puncture. After lysis of the red blood cells, the blood was stained for immune phenotype markers (CD3, CD4, CD8, NKp46, NK1.1, FOXP3, CD44, and CD62L), as well as Ki67, and a viability marker. Cells were analyzed using an Attune NXT flow cytometer. The concentration of CD4+ FOXP3− T cells, CD8+ T cells, NK cells, and Tregs within each sample was calculated. The percentage of CD4+ FOXP3− T cells, CD8+ T cells, NK cells, and Tregs which were Ki67+ was calculated. The percentage of CD4+ FOXP3− T cells and CD8+ T cells which were Naïve, Central Memory of Effectors was calculated.

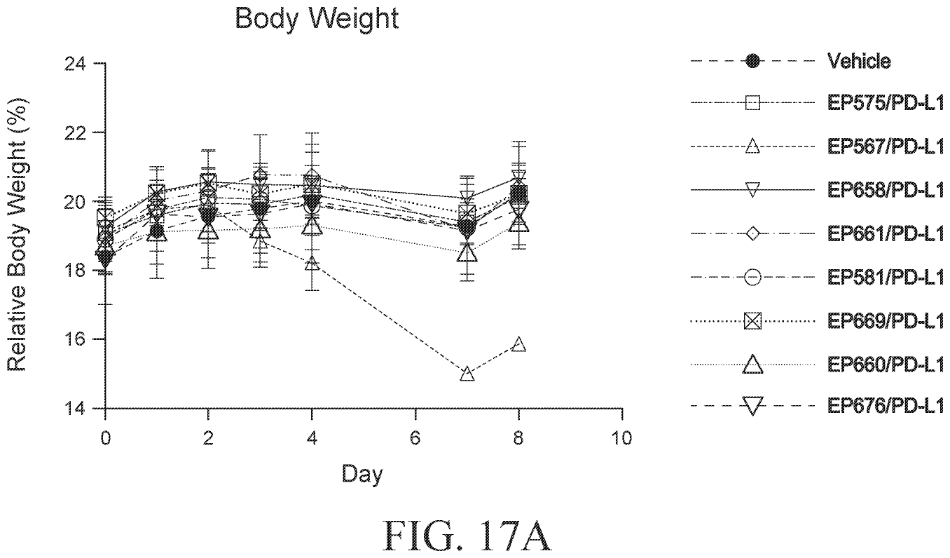

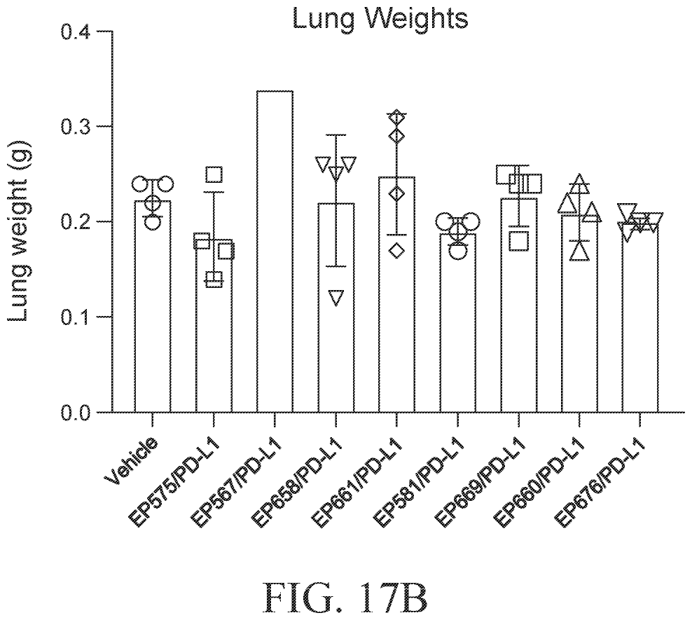

FIG. 17A a showed the body weight change of the mice. Consistent with the previous results, EP567/anti-PD-L1 bispecific induced significant body weight loss at 10 mg/kg, and death at 10 mg/kg with increased lung weight (FIG. 17B). In contrast, all other tested IL2/PD-L1 bispecific antibodies, including EP575/anti-PD-L1, EP581/anti-PD-L1, EP658/anti-PD-L1, EP660/anti-PD-L1, EP661/anti-PD-L1, and EP669/anti-PD-L1 showed no body weight loss (FIG. 17A). Accordingly, no apparent change of the lung weight (FIG. 17B).

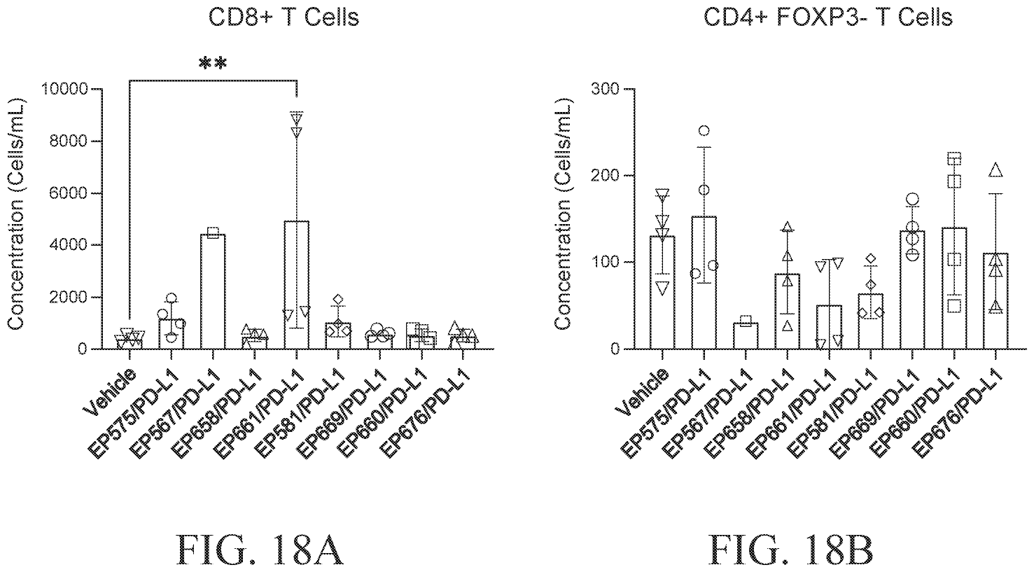

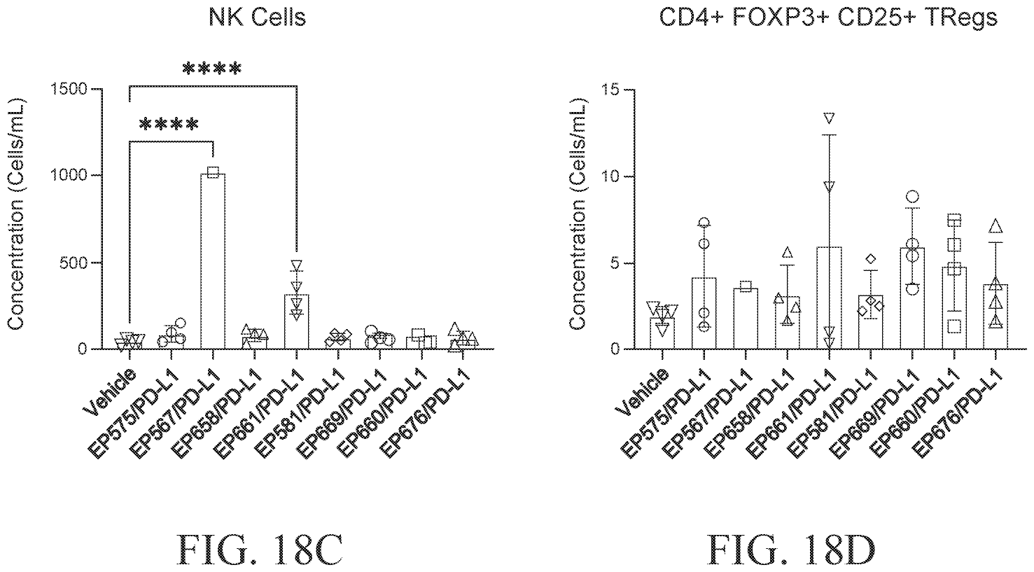

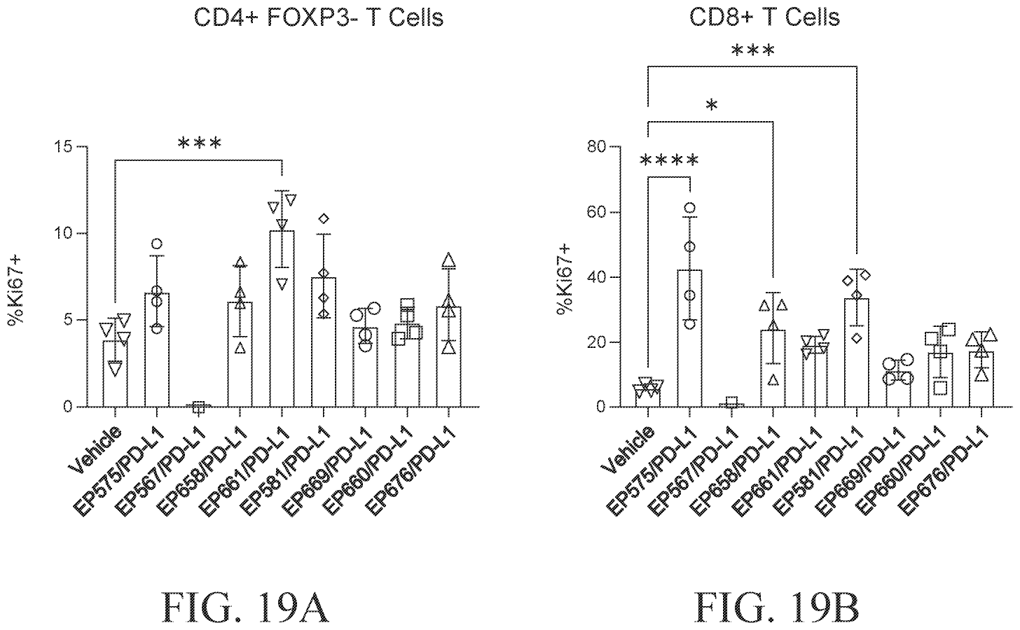

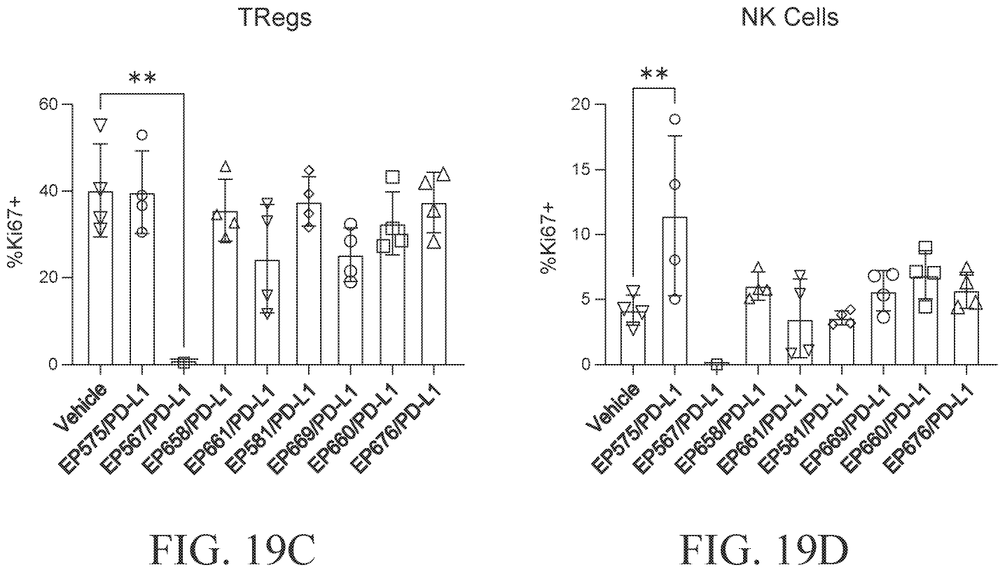

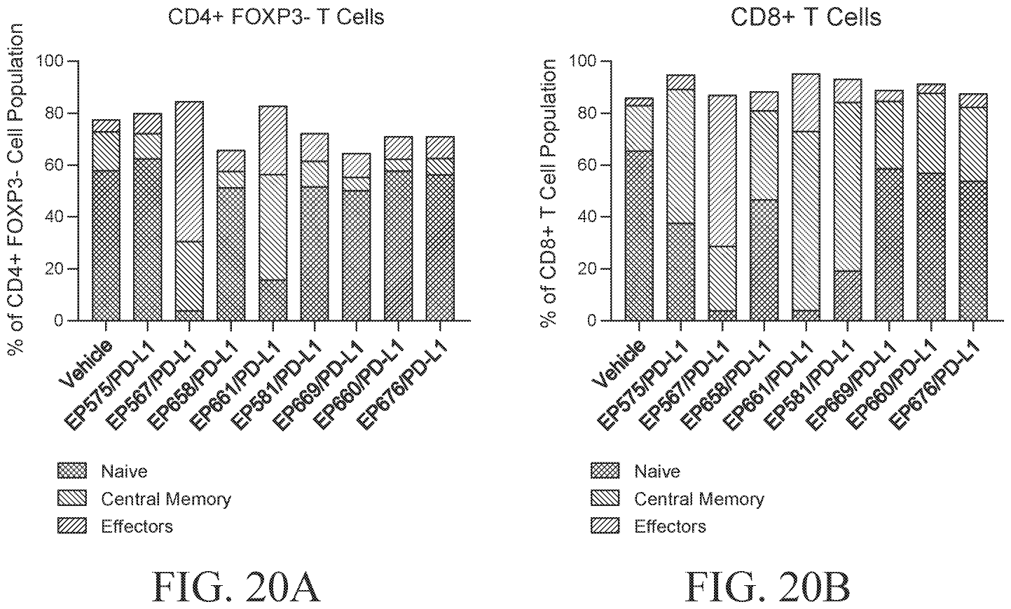

Peripheral immunophenotyping profiling was conducted to assess whether those IL2/anti-PD-L1 antibodies (e.g., bifunctional fusion protein complex) could modulate immune cell activations. Only one mouse in EP567/anti-PD-L1 was survived. The profiling results for this group were consistent with the previous data. Significantly increased CD8 T cells, NK cells and reduced Treg cells were observed in this group (FIG. 18). Among the rest of IL2/anti-PD-L1 bispecifics, EP661/anti-PD-L1 showed significant increase of CD8 and NK cell activation with a modest increase of Treg. EP575/anti-PD-L1 and EP581/anti-PD-L1 showed modest activation of CD8 T cells, NK cell and Treg cells (FIG. 18). Whereas EP658/anti-PD-L1, EP660/anti-PD-L1, EP669/anti-PD-L1, EP676/anti-PD-L1 showed weak or minimal activation of CD8 T cells and NK cells. Treg cells in those three groups were increased modestly. Interestingly, there was reduced proliferation of immune cells in EP567/anti-PD-L1 group (FIG. 19). EP575/anti-PD-L1, EP581/anti-PD-L1 and EP658/anti-PD-L1 showed significant increased proliferation of CD8 T cells (FIG. 19B). EP660/anti-PD-L1, EP661/anti-PD-L1, EP669/anti-PD-L1 and EP676/anti-PD-L1 showed modest CD8 T cell proliferation (FIG. 19B). Only EP575/anti-PD-L1 showed significantly increased NK cell proliferation (FIG. 19D). Subtype immune cell profiling results suggested that EP567/anti-PD-L1 group contains more effective CD4 and CD8 T cells and small percentage of Naïve T cells. EP661/anti-PD-L1 showed similar pattern as EP567/anti-PD-L1. EP575/anti-PD-L1, EP581/anti-PD-L1 and EP661/anti-PD-L1 contain high level of central memory CD8 T cells. EP660/anti-PD-L1, EP669/anti-PD-L1 and EP676/anti-PD-L1 contains high level of naïve CD8 T cells similar to that of vehicle, further suggesting their weak or modest systemic immune modulating activities (FIG. 20). Table 9 listed the average concentration fold change of immune cells in peripheral blood of IL2/anti-PD-L1 treated mice over the vehicle group mice.

TABLE 9
Concentration fold change of immune cells in peripheral
blood compared to the vehicle group in mice.

| | Average Fold Change of Concentration vs Vehicle | | | | | |
|---|---|---|---|---|---|---|
| | CD45 | CD3 | CD8 | NK | CD4 | T reg |
| Vehicle | 1 | 1 | 1 | 1 | 1 | 1 |
| EP575/PDL1 | 1.37 | 2.16 | 2.76 | 1.89 | 1.17 | 2.16 |
| EP567/PDL1 | 3.32 | 6.35 | 10.35 | 21.65 | 0.24 | 1.86 |
| EP658/PDL1 | 0.84 | 1.07 | 1.27 | 1.18 | 0.68 | 1.63 |
| EP661/PDL1 | 2.54 | 7.42 | 11.49 | 6.90 | 0.39 | 3.06 |
| EP581/PDL1 | 0.96 | 1.70 | 2.49 | 1.46 | 0.49 | 1.64 |
| EP669/PDL1 | 1.16 | 1.31 | 1.40 | 1.41 | 1.04 | 3.04 |
| EP660/PDL1 | 1.02 | 1.27 | 1.27 | 1.09 | 1.075 | 2.49 |
| EP676/PDL1 | 1.059 | 1.14 | 1.25 | 1.39 | 0.85 | 1.96 |

### Example 14

### Pharmacokinetics Analysis of EP661/Anti-PD-L1 Agonist in Mouse

Female C57BL/6 mice aged 6-8 weeks were given a single 200 μg intravenous (iv) bolus dose of EP661/anti-PD-L1. Mice were sacrificed by terminal cardiac puncture at the following timepoints: pre-dose, 1 hour, 2 hours, 6 hours, 1 day, 3 days, 7 days, and 10 days after initial dosing. Blood was collected in Lithium Heparin tubes, spun down, and plasma was collected and stored at −80° C. until analysis.

Once all time-points had been collected, the plasma was thawed for analysis. Anti-human Fc was diluted in 25 μL of 1× PBS and added to wells of a 384-well immunosorbent plate and incubated overnight at 4° C. to coat the plate. The plate was blocked with 100 μL of SuperBlock for 1 hour at room temperature. Plasma samples were diluted 1:1000 or 1:2000 in PBS and added to the wells in duplicates. A known concentration of EP661/anti-PD-L1 was serially diluted in PBS containing 0.1% mouse plasma, creating a standard, and added to wells in duplicate. Samples and standards were incubated for 1 hour at room temperature. Bound EP661/anti-PD-L1 was sandwiched with biotinylated recombinant PD-L1 and detected by adding 25 μL of streptavidin HRP diluted at 1:5000 in 1× PBST. In between each step, the plate was washed three times with 1×PBST using a plate washer. The plate was then developed with 25 μL of TMB substrate for five minutes and stopped by adding 25 μl of 2N sulfuric acid. The plate was read at OD450 nm Biotek plate reader.

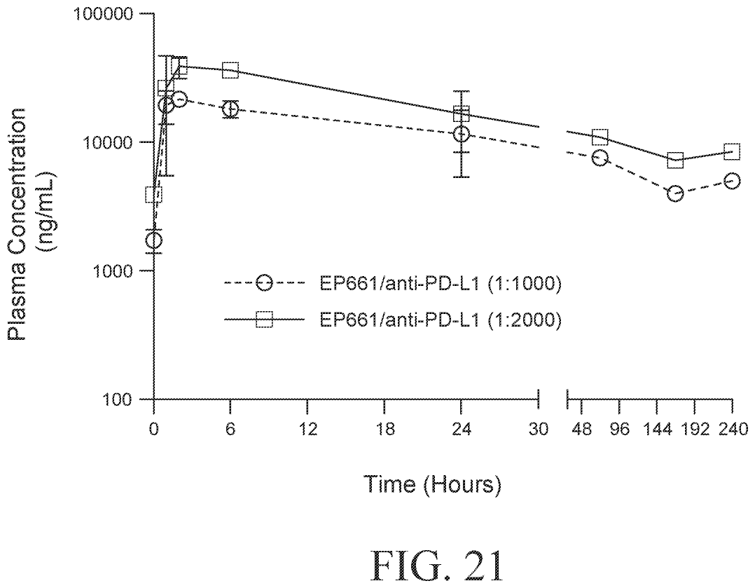

The OD values from the standards were plotted in Microsoft Excel against the known concentrations, and line of best fit was drawn by performing a simple linear regression. The EP661/anti-PD-L1 concentrations within the samples were calculated from the OD values based on the equation from the standard's line of fit and multiplied by the initial 1:1000 dilution factor. Protein concentrations were plotted against time. PK parameters were calculated using PKSolver software and Microsoft Excel, and displayed in FIG. 21. Table 10 showed the calculated PK parameters.

TABLE 10
PK parameters of EP661/anti-PD-L1 in mouse.

| Parameter | 10 mg/kg |
|---|---|
| T1/2 (h) | 82.56 |
| Cmax (ng/ml) | 19903.87 |
| Cl_obs (mg/kg/h) | 5.49107E−06 |
| Vss_obs (mg/kg) | 0.000732028 |

### Example 15

### IL2RB Agonist Anti-Tumor Activities in Mice Tumor Model

6-8-week old, female C57BL/6 mice were injected with 500,000 B16-F10-hPD-L1 cells subcutaneously on their back flank. Tumors were measured with calipers. Upon reaching an average volume of ˜50 mm³, mice were treated with 10 mg/kg IL2/anti-PD-L1 bispecific (e.g., bifunctional fusion protein complex) or Atezolizumab Q4D. Tumors were measured 3 times per week, and body weight was measured 5 times per week.

On Day 8, peripheral blood was collected. After lysis of the red blood cells, the blood was stained for immune phenotype markers (CD3, CD4, CD8, NKp46, NK1.1, and FOXP3), as well as a viability marker. Cells were analyzed using an Attune NxT flow cytometer. The number of CD4+ FOXP3− T cells, CD8+ T cells, NK cells, and Tregs within each sample was calculated. The ratio of CD8+ T cells and NK Cells relative to the number of Tregs was calculated.

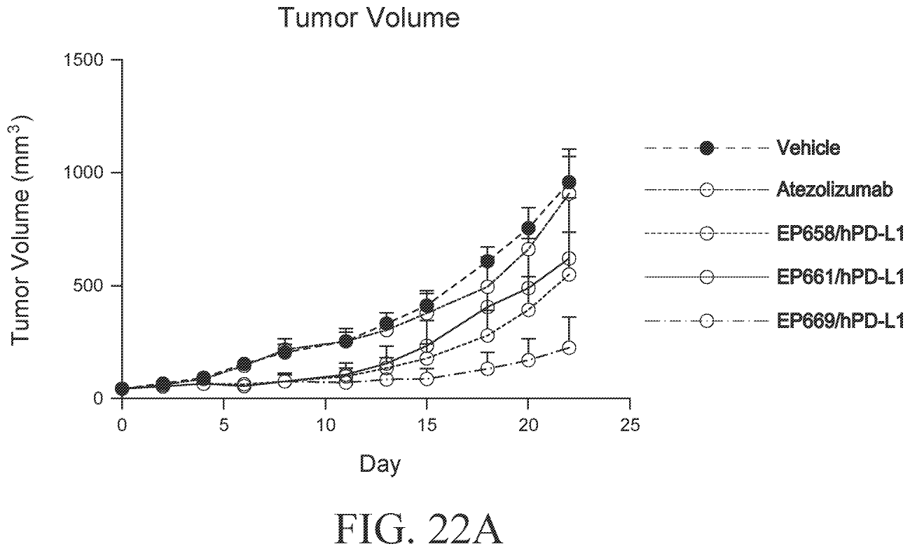

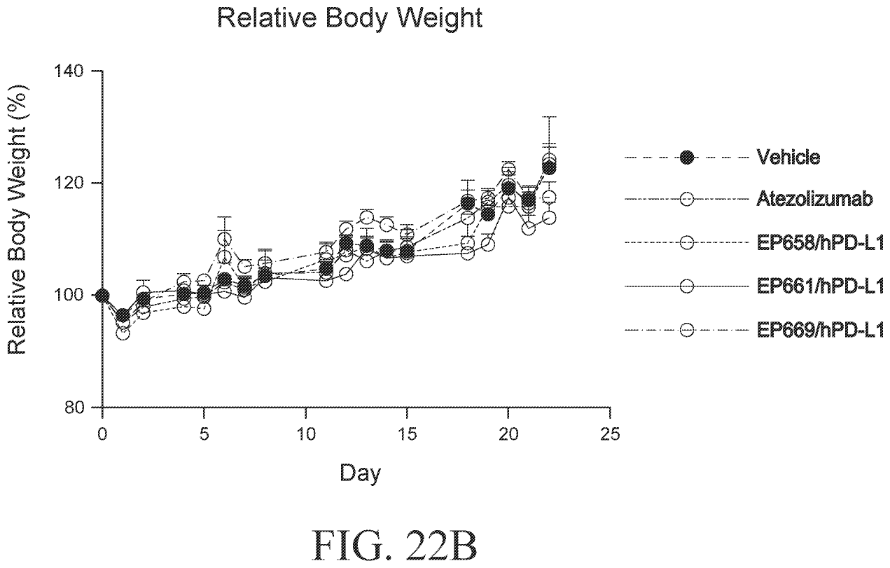

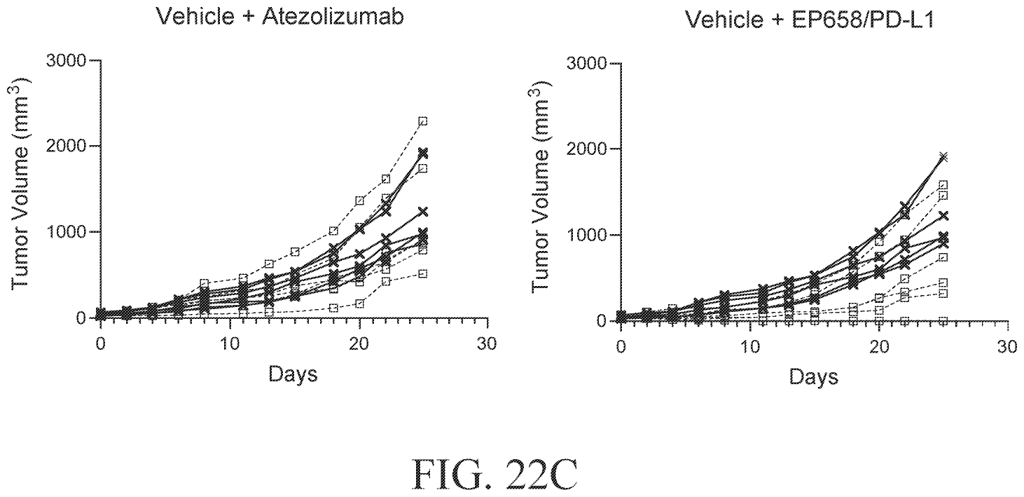

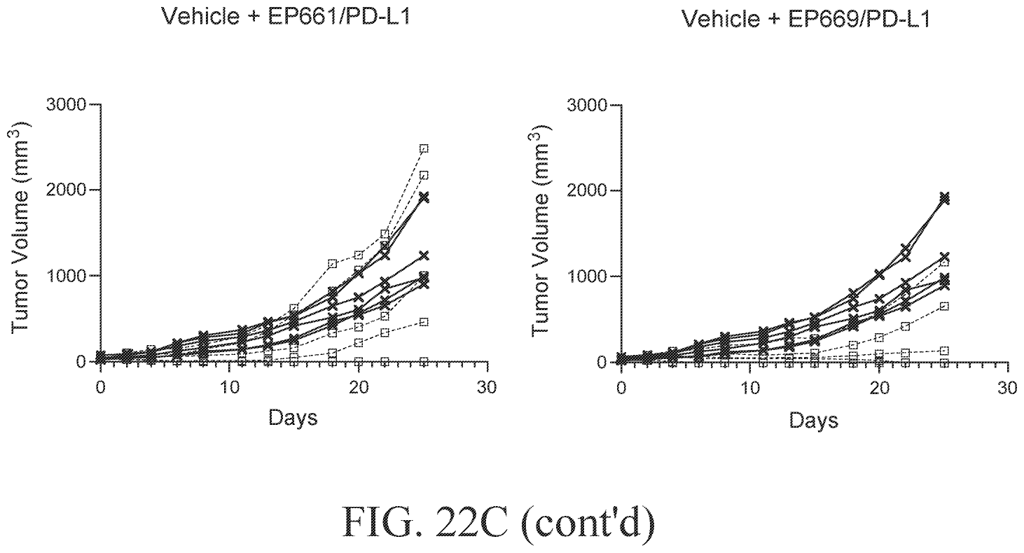

FIG. 22A shows the tumor growth curves of EP658/anti-PD-L1, EP661/anti-PD-L1 and EP669/anti-PD-L1 (Q4D, 200 μg) in comparison with atezolizumab (Q4D, 200 μg) and vehicle (1×PBS, Q4D). FIG. 22B shows the body weight change of the mice during the treatment. Consistent with previous studies, mice were well tolerating to the repeated dose of all study groups. All IL2/anti-PD-L1 treated groups showed stronger tumor growth inhibition activities than that of atezolizumab. Some of the mice experienced complete tumor regression in IL2/anti-PD-L1 groups. Among them, one mouse was tumor free in EP658/anti-PD-L1, two mice in EP661/anti-PD-L1 and 3 mice in EP669/anti-PD-L1 group were tumor free. EP669/anti-PD-L1 showed strongest anti-tumor activities with tumor growth inhibition of 69%. FIG. 22C shows the individual mouse tumor growth curve. In addition to the consistently improved safety, this early tumor model study supported that the IL2Rβ reduced-binding IL2 in bispecific with anti-PD-L1 may synergize to improve the anti-tumor activities.

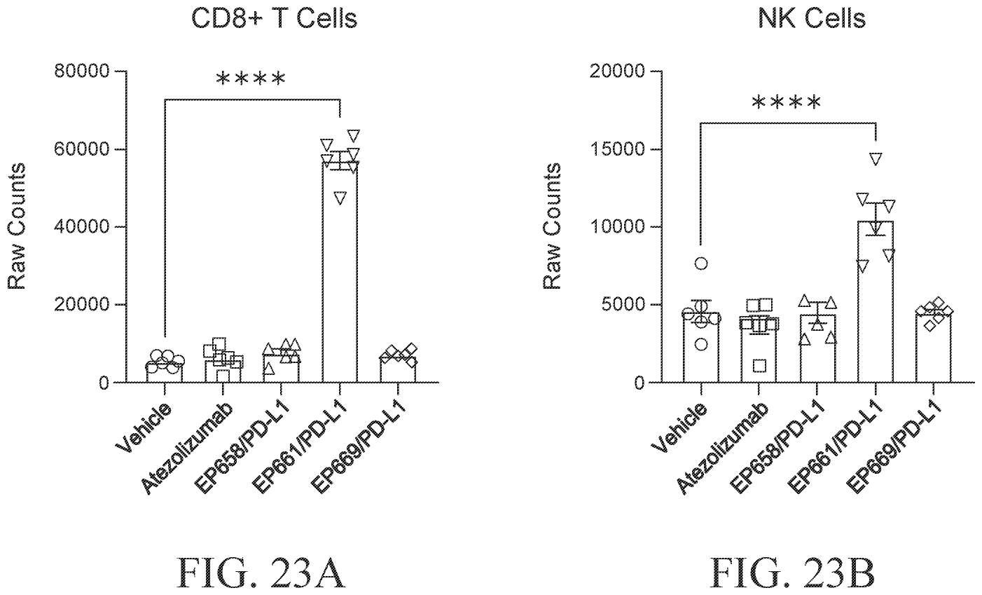

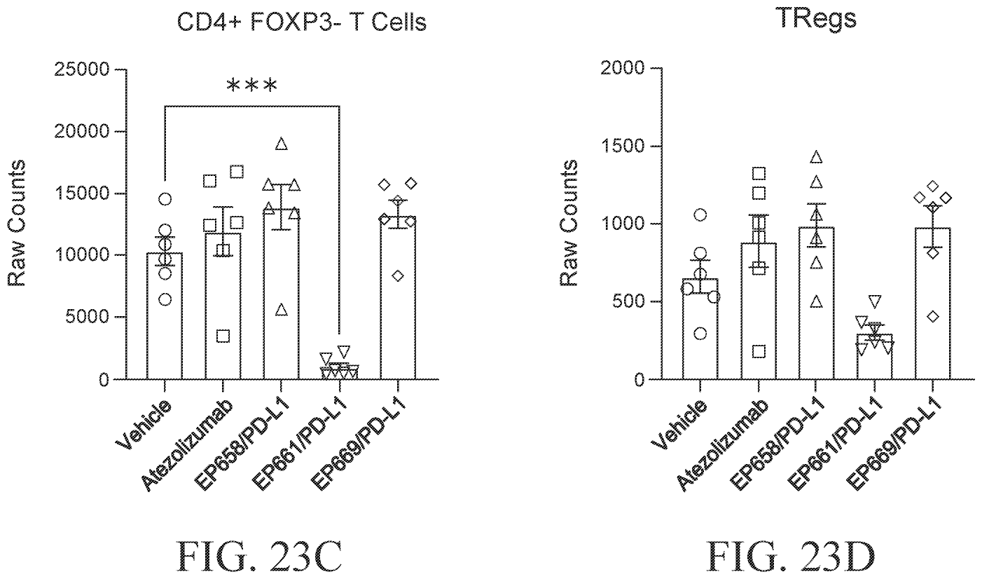

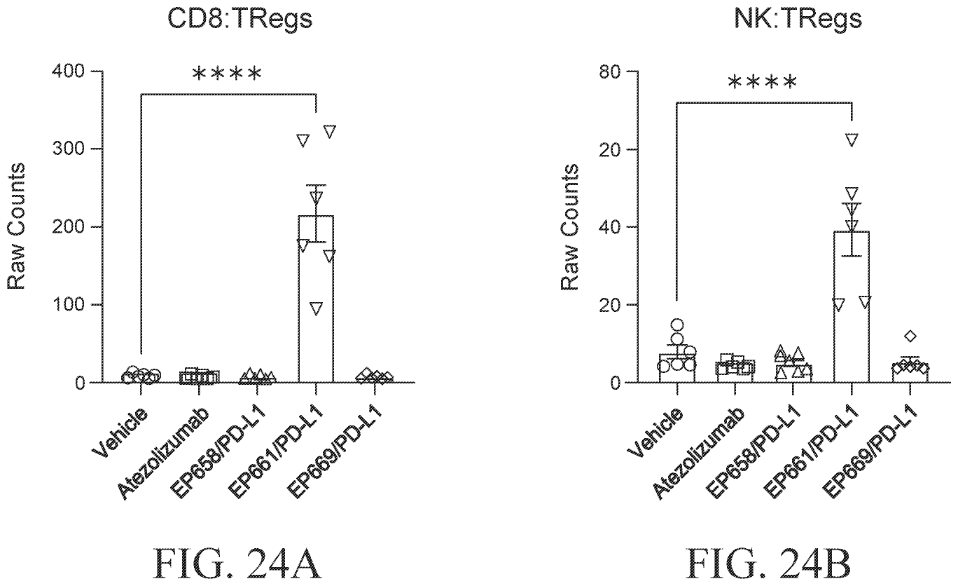

The immunophenotyping analysis of peripheral blood collected on day 8 (prior to third does) suggested consistent results from previous studies. Significant CD8 T cells and NK cell activation were observed for EP661/anti-PD-L1, whereas EP658/anti-PD-L1 and EP669/anti-PD-L1 induced modest CD8 and NK cells activation. Treg cells also were modestly activated in those two groups. In agreement with previous studies, EP661/anti-PD-L1 reduced the Treg and CD4 cells (FIG. 23). EP661/anti-PD-L1 showed significant increase of ratio of CD8:Treg and NK:Treg in peripheral blood (FIG. 24), whereas the ratio increment in EP658/anti-PD-L1 and EP669/anti-PD-L1 group was minimal. Table 11 showed average raw count fold change of immune cells in peripheral blood of IL2/anti-PD-L1 treated groups over the vehicle group. The immunophenotyping analysis further supported that EP661 is a strong CD8 and NK cell activation modulator, whereas EP669 and EP658 are mild to modest systemic CD8 and NK cell activation modulators.

TABLE 11
Raw count fold change of immune cells in peripheral blood
of IL2/anti-PD-L1 treated groups over the vehicle group.

Fold Change vs Vehicle, raw count

| | CD8 | CD4 | NK | Treg |
|---|---|---|---|---|
| Vehicle | 1 | 1 | 1 | 1 |
| Atezolizumab | 1.23 | 1.44 | 0.86 | 1.23 |
| EP658/PD-L1 | 1.69 | 1.78 | 1.19 | 1.58 |
| EP661/PD-L1 | 14.93 | 0.65 | 3.2 | 0.14 |
| EP669/PD-L1 | 1.52 | 1.69 | 1.15 | 1.47 |

### Example 16

### Anti-Tumor Activities of IL2/PD1 and IL2/PD-L1 Bispecifics in Tumor Models

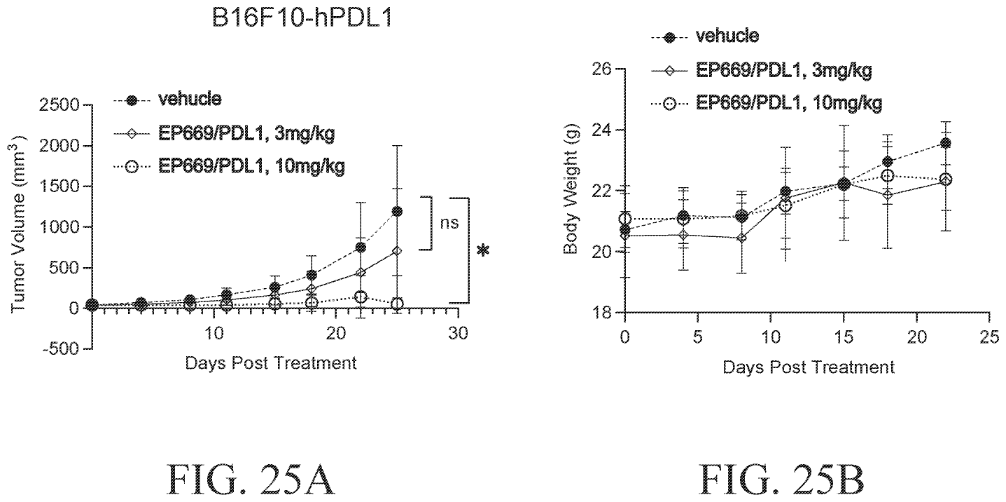

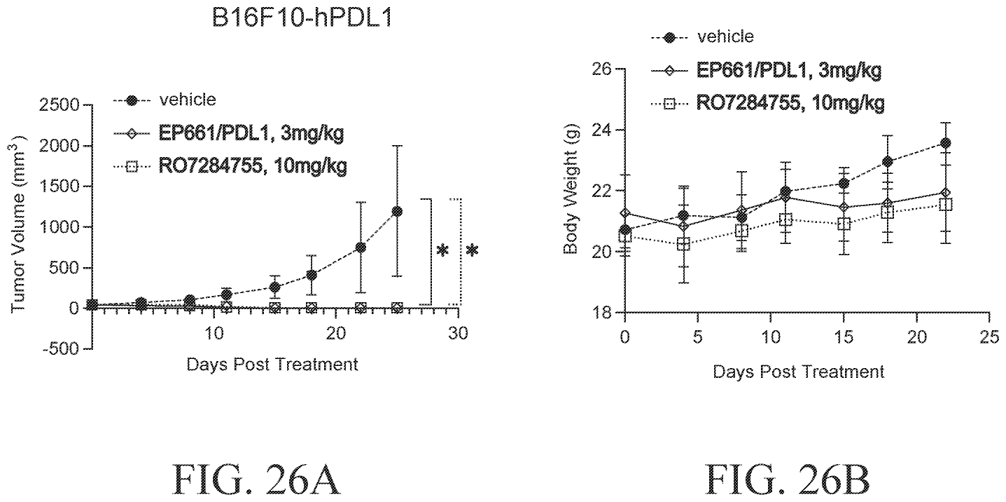

To evaluate the anti-tumor activity of IL-2/PD1 and PD-L1 bispecifics (e.g., bifunctional fusion protein complex), 6-8 week old, female C57BL/6 mice were injected with 500,000 B16-F10-hPD-L1 cells subcutaneously on their hind flank. Tumors were measured with calipers. Upon reaching an average volume of ˜50 mm³, mice were treated with EP669/PDL1 at either 3 or 10 mg/kg (both Q4D), EP661/PD-1 3 mg/kg QW, or RO7284755 1 mg/kg QW. Mouse body weight was measured 2 times per week and plotted against time for each group. Mean tumor volume and individual tumor volume was plotted for each group against time (FIGS. 25-26).

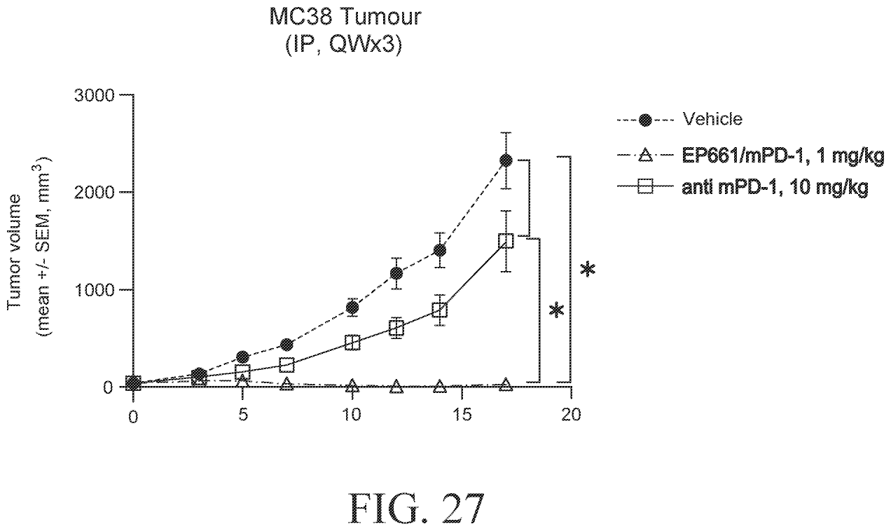

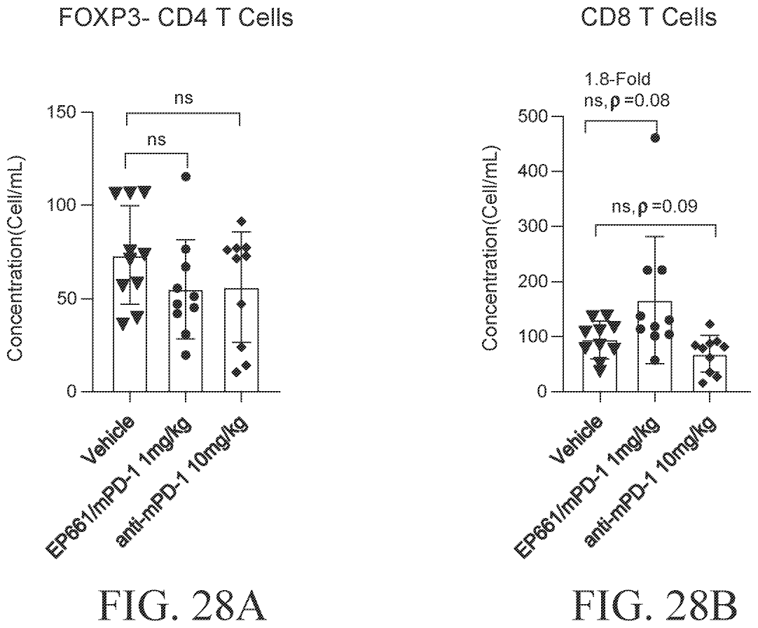

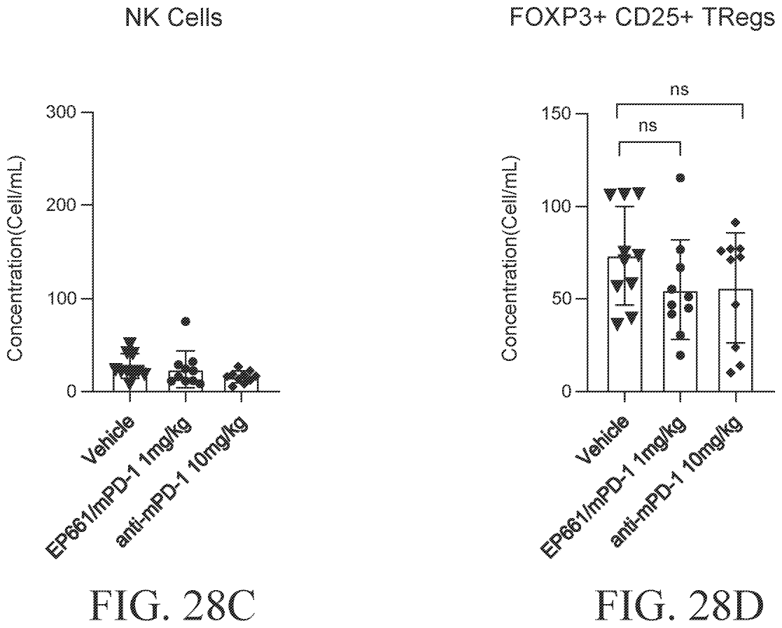

Female, C57B1/6 mice (6-8 weeks old) were inoculated subcutaneously in the right hind flank with 2E5 MC38 tumor cells suspended in 200 μl PBS. Tumor volume was measured using calipers and mice were randomized to three treatment groups when tumor volume reached ˜50 mm³. Mice were treated with vehicle (PBS), EP661/mPD-1 at 1 mg/kg or anti-mPD-1 at 10 mg/kg. Treatments were administered by intra-peritoneal injection once weekly for 2 weeks. Tumor volume was measured twice weekly for the duration of the study. Percent tumor growth inhibition (TGI) relative to vehicle was calculated on Day 17, and animals that demonstrated a complete regression (CR) were determined (FIG. 27, Table 12). On study day 11, peripheral blood was collected. After lysis of the red blood cells, the remaining cells were stained for immune phenotype markers (CD3, CD4, CD8, NKp46, NK1.1, FOXP3,), as well as a viability marker. Cells were analyzed using an Attune NXT flow cytometer. The concentration of CD4+ FOXP3− T cells, CD8+ T cells, NK Cells, and CD4+ FOXP3+ TRegs within each sample was calculated and plotted (FIGS. 28A-28D).

TABLE 12
Tumor Growth Inhibition

| | TGI (%) | CR |
|---|---|---|
| Vehicle | — | 0/10 |
| Anti-mPD1 | 43 | 0/10 |
| EP661/mPD1 | 99 | 8/10 |

### Example 17

### IL2RB Agonist Immune Cell Modulation Activity

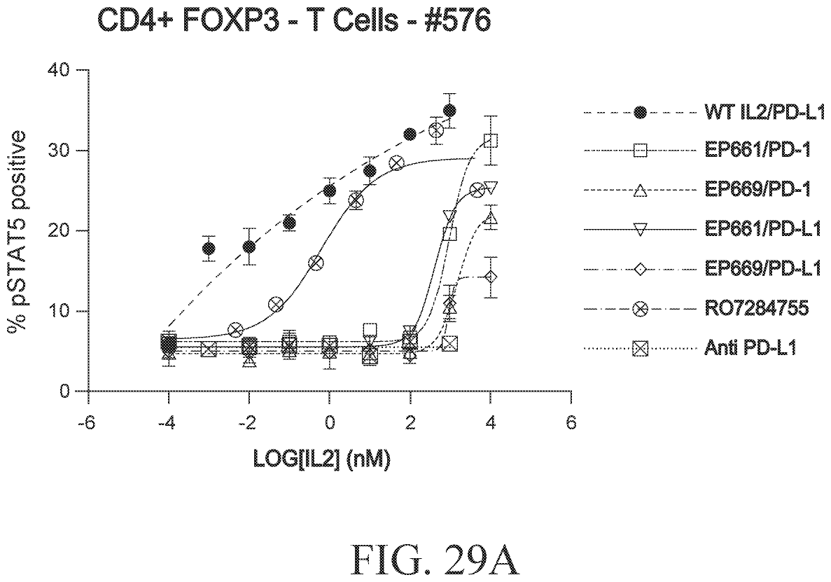

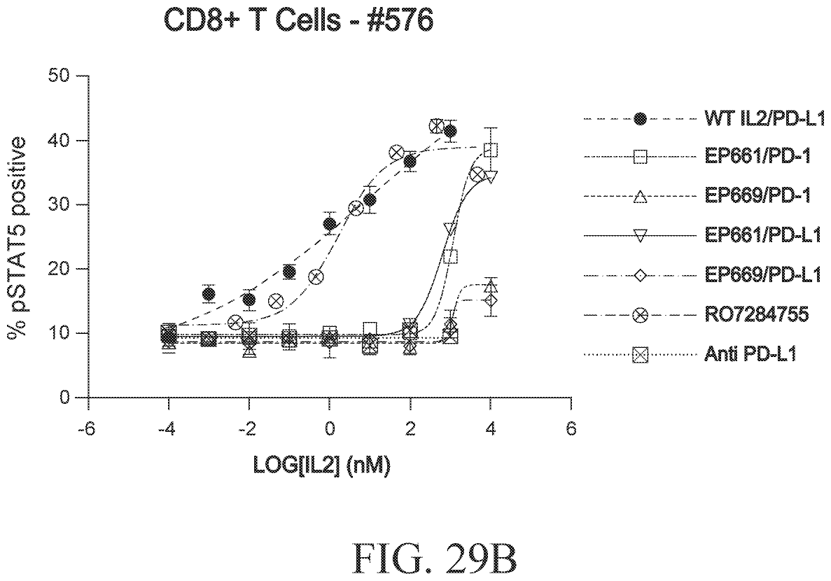

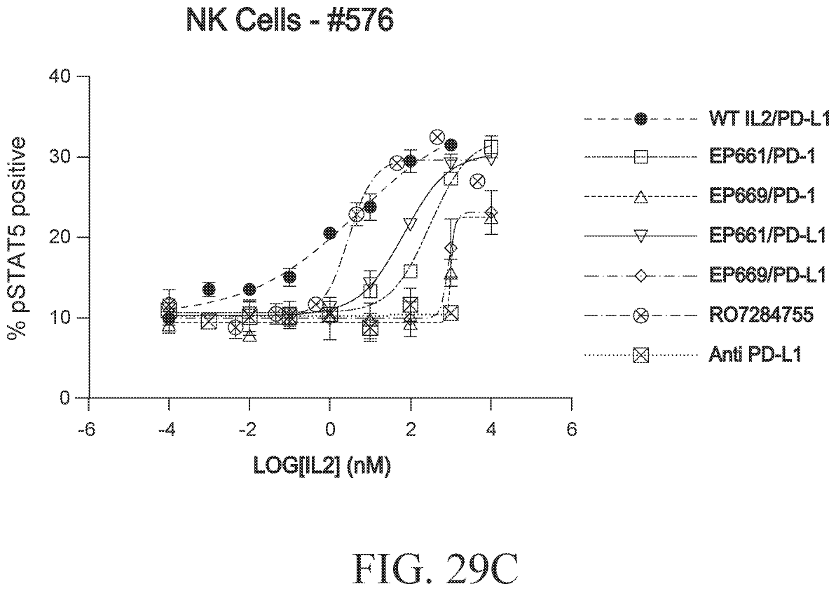

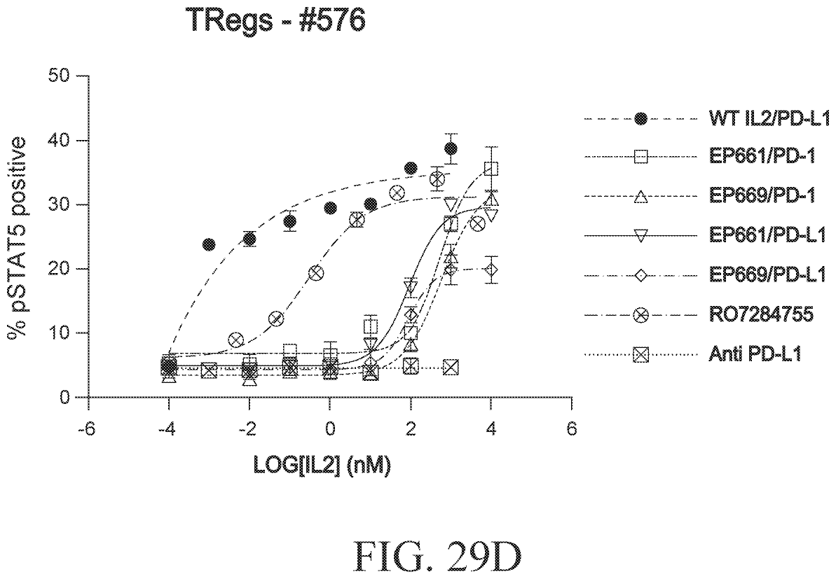

To characterize the immune cell modulation activities of the reduced activity IL2 mutant bispecifics (e.g., bifunctional fusion protein complex) compared to WT IL2 and RO7284755, Human PBMCs were isolated from peripheral blood, stained with viability dye, and plated at 250,000 cells/well in a 96-well plate in 90 μL of media. Cells were rested 1 hr at 37° C. Cells were stimulated with a titration of WT IL2 bispecific and reduced IL2 mutant bispecifics at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD4+ FOXP3− T cells were defined as CD3+CD56−CD4+CD8−FOXP3−. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL2 titration (FIG. 29). P-STAT5 activation were determined using Prism software. Those data supported that the engineered IL2 showed reduced binding to IL2Rα and IL2Rβ receptors. Correspondingly, the engineered IL2s showed weak p-STAT5 phosphorylation activities with incomplete curve or low activities at high concentrations compared to that of WT IL2 and RO7284755 (FIG. 29).

### Example 18

### IL2RB Agonist Activity on Tumor Infiltrating Cells

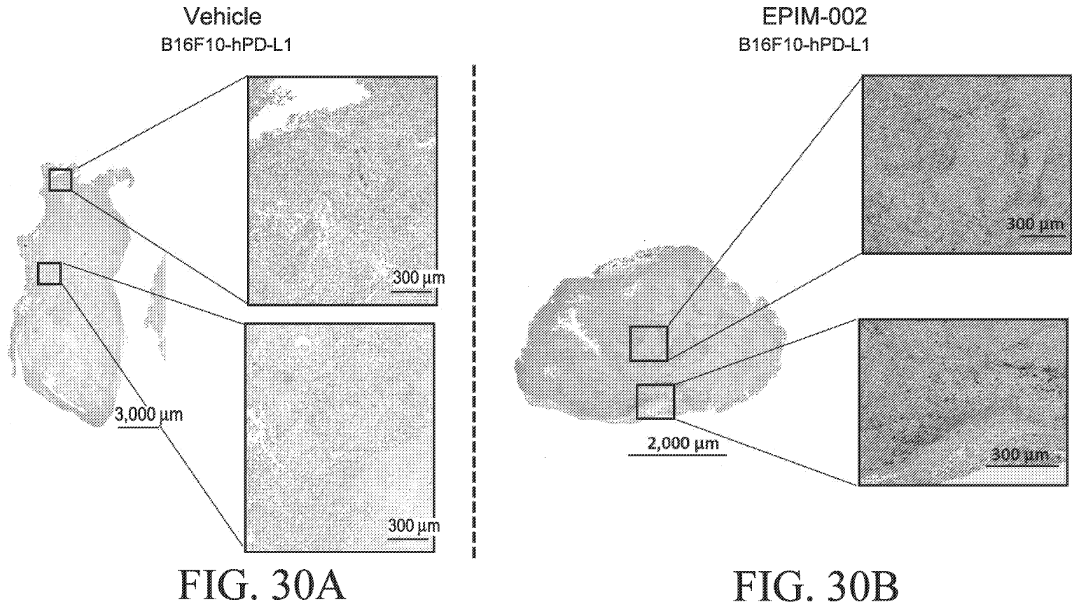

6-8-week old, female C57BL/6 mice were injected with 500,000 B16-F10-hPD-L1 cells subcutaneously on their hind flank. Tumors were measured with calipers. Upon reaching an average volume of ˜50 mm³, mice were treated with vehicle or EP669/PDL1 (3 mg/kg, Q4D). At the study endpoint, animals were euthanized by CO₂ asphyxiation and tumors were excised. Tumors were fixed in formalin and embedded in paraffin. Tumors blocks were then cut to 5 μm thickness on glass slides and processed for IHC staining. Slides were blocked with BLOXALL (Vector, SP-6000-100) and 2.5% normal horse serum (Vector S-2012-50) before incubation at room temperature for 1 hr with 1:1000 dilution of CD8α antibody (CST, 98941) in Signal Stain (CST, 8112L). After washing with TBS-T, slides were incubated for 1 hr with goat anti-rabbit IgG Amplifier (Vector, MP-7601-50). Slides were washed in TBS-T before incubation with ImmPress HRP Horse Anti-Goat IgG Polymer Reagent (Vector, MP-7601-50. After washing, ImmPACT DAB EqV chromogen solution (Vector, MP-7601-50) was applied and slides were incubated. All incubations were performed at room temperature. Slides were rinsed in TBS-T and distilled water, counterstained with Gill II Hematoxylin (StatLab, SL94-1), dehydrated and covered with coverslips. Slides were then imaged and representative areas of untreated and treated tumors were magnified to visualize CD8-positive cells (FIG. 30).

### Example 19

### IL2RB Agonist Activity on Systemic Immune Cells

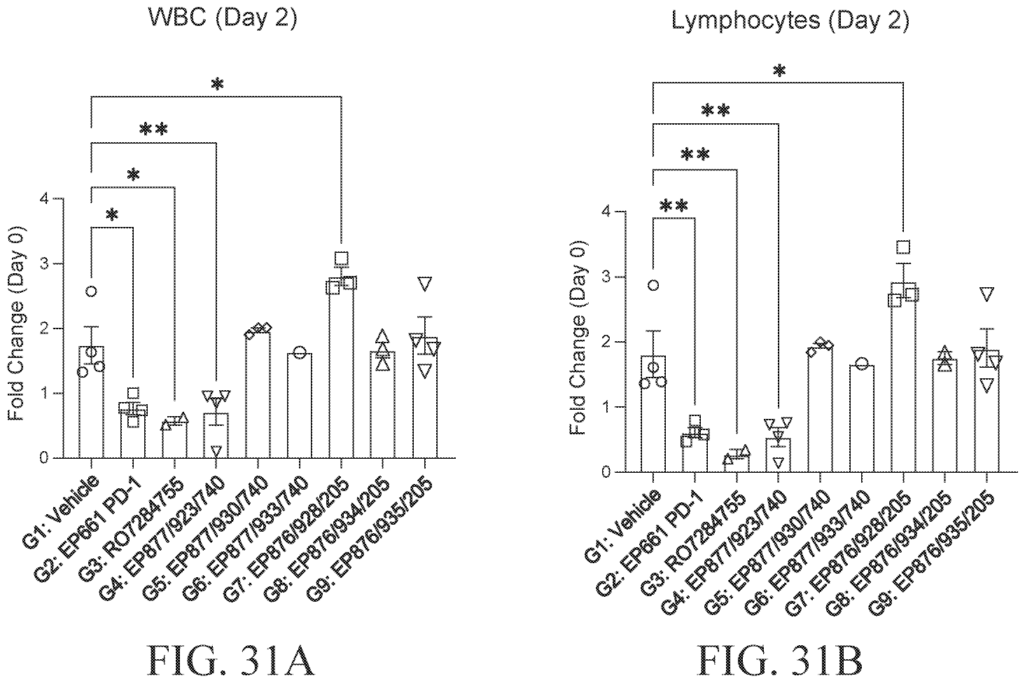

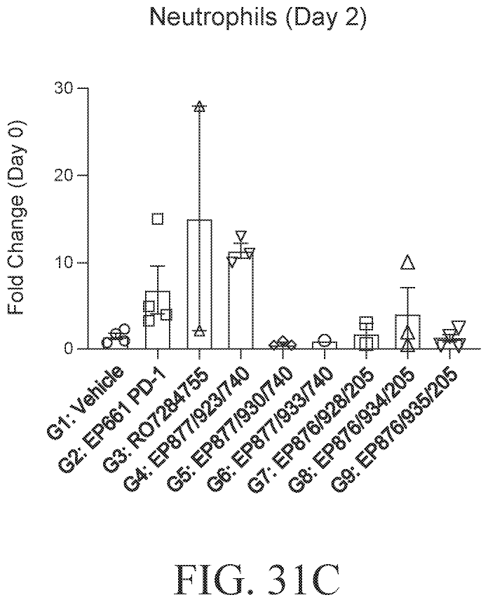

To evaluate the activity of IL2 bispecifics (e.g., bifunctional fusion protein complexes) on systemic immune cells, blood was collected by submandibular bleeds from naïve female C57B1/6 mice (6-8 weeks old) and complete blood counts were measured using the VETSCAN hematology analyzer (Abaxis). Mice were then treated with vehicle (PBS), RO7284755 or IL2 mutant bispecific molecules (EP877/EP923/EP740; EP877/EP930/EP740; EP877/EP933/EP740; EP876/EP928/EP205; EP876/EP934/EP205; or EP876/EP935/EP205). Treatments were administered by intra-peritoneal injection at a dose volume of 200 μl for a dose concentration of 200 μg/mouse. Two days after treatment, blood was again collected from all animals for complete blood count analysis. Fold change in total white blood cells (WBC), lymphocytes and neutrophils from pre-treatment (Day 0) to day 2 blood samples are graphed (FIG. 31). The data supported a stronger lymphopenia effect for RO7284755 than the IL2 mutant bispecific molecules disclosed herein.

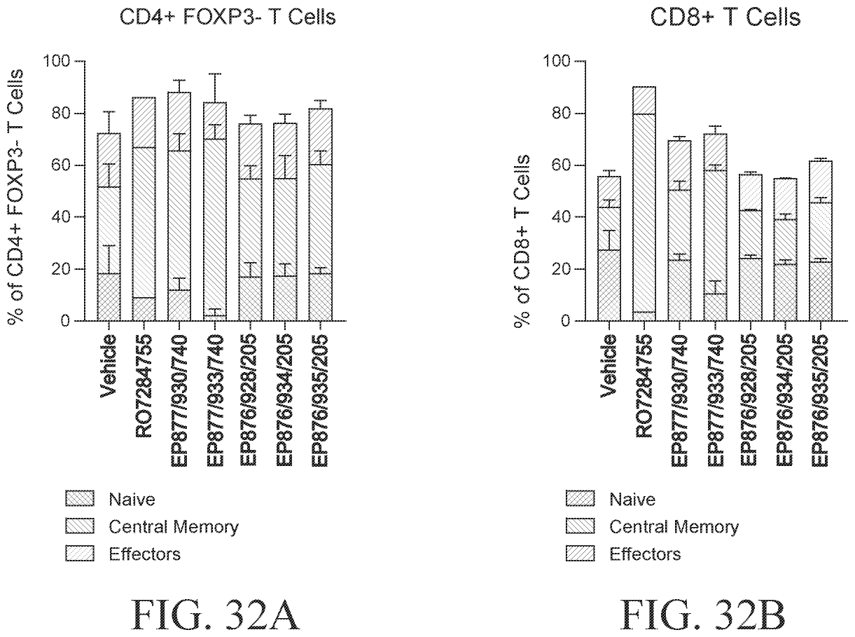

On Day 5, peripheral blood was collected. After lysis of the red blood cells, the remaining cells were stained for immune phenotype markers (CD3, CD4, CD8, NKp46, NK1.1, FOXP3, CD44, CD62L), as well as a viability marker. Cells were analyzed using an Attune NxT flow cytometer. CD4+ FOXP3− T cells were identified as: CD3+, NKp46−, NK1.1−, CD8−, CD4+, FOXP3−. CD8+ T cells were identified as: CD3+, NKp46−, NK1.1−, CD8+, CD4−. From these two populations, the percentage of Naïve (CD44−, CD62L+), Central Memory (CD44+, CD62L+), and Effectors (CD44+, CD62L−) of the parent populations was determined and plotted (FIG. 32).

### Example 20

### Anti-Tumor Activities of IL2/PD1 Bispecifics in Tumor Models

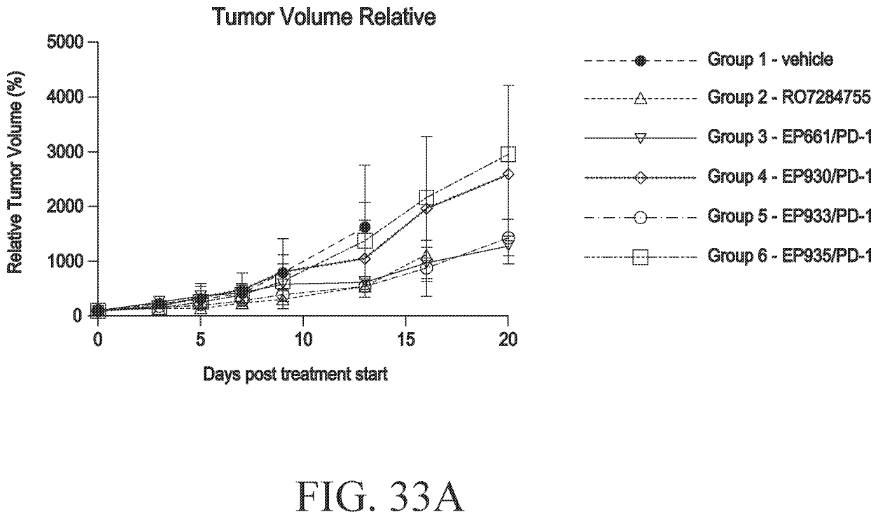

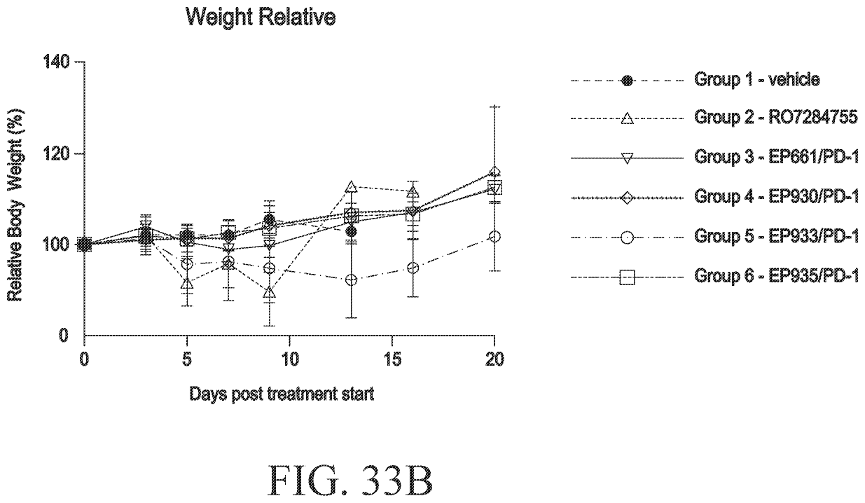

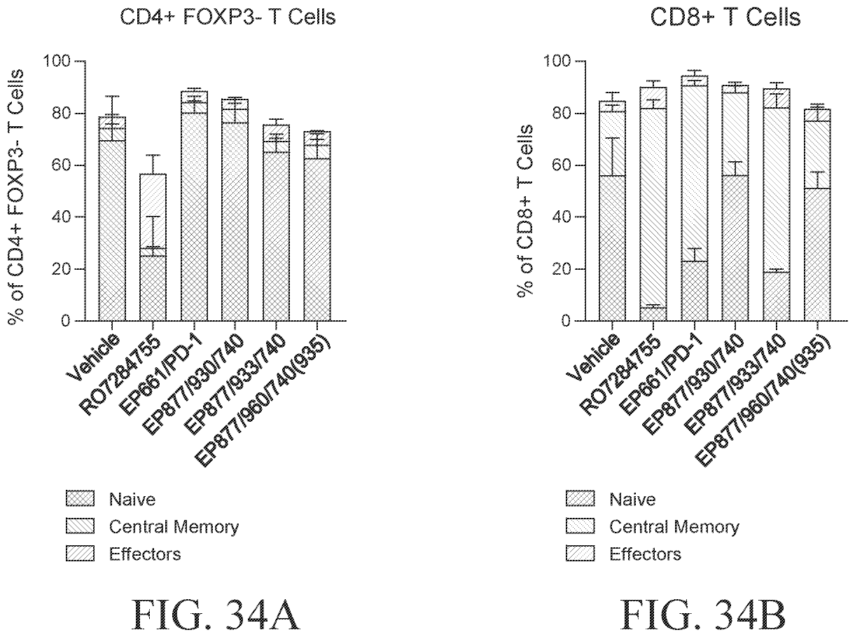

Female, C57B1/6 mice (6-8 weeks old) were inoculated subcutaneously in the right hind flank with 2E5 MC38 tumor cells suspended in 200 μl PBS. Tumor volume was measured using calipers and mice were randomized to three treatment groups when tumor volume reached ˜50 mm³. Mice were treated with vehicle (PBS), RO7284755, EP661/mPD-1, EP930/PD-1, EP933/PD-1, or EP935/PD-1 at 1 mg/kg. Treatments were administered by intra-peritoneal injection once weekly for 2 weeks. Tumor volume was measured twice weekly for the duration of the study. Percent tumor growth inhibition (TGI) relative to vehicle was calculated on Day 17, and animals that demonstrated a complete regression (CR) were determined (FIG. 33). On study day 11, peripheral blood was collected. After lysis of the red blood cells, the remaining cells were stained for immune phenotype markers (CD3, CD4, CD8, NKp46, NK1.1, FOXP3,), as well as a viability marker. Cells were analyzed using an Attune NxT flow cytometer. CD4+ FOXP3− T cells were identified as: CD3+, NKp46−, NK1.1−, CD8−, CD4+, FOXP3−. CD8+ T cells were identified as: CD3+, NKp46−, NK1.1−, CD8+, CD4−. From these two populations, the percentage of Naïve (CD44−, CD62L+), Central Memory (CD44+, CD62L+), and Effectors (CD44+, CD62L−) of the parent populations was determined and plotted (FIG. 34).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/340,294, filed May 10, 2022, and U.S. Provisional Patent Application No. 63/391,243, filed Jul. 21, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An engineered interleukin-2 (IL2) polypeptide comprising an engineered IL2 receptor β (IL2Rβ) binding region 2 motif comprising:
(SEQ ID NO: 3)
X₁-X₂-X₃-X₄-X-₅-X₆-X₇-X₈-X₉-X₁₀-X₁₁-X₁₂-X₁₃-
X₁₄-X₁₅-X₁₆,
X₁ comprises a residue selected from R, S, L, N, F, K, or T;
X₂ comprises a residue selected from A, F, S, L, R, T, I, H, P, or N;
X₃ comprises a residue selected from K, R, T, S, I, or P;
X₄ comprises a residue selected from G, D, R, A, Q, H, N, Y, or E;
X₅ comprises a residue selected from I, P, T, S, K, F, V, or L;
X₆ comprises a residue selected from I, R, V, M, T, or L;
X₇ comprises a residue selected from A, R, M, I, S, N, G, or S;
X₈ comprises a residue selected from E, N, H, T, K, Y, S, L, V, D, or R;
X₉ comprises a residue selected from I, V, A, T, L, T, or M;
X₁₀ comprises a residue selected from N, G, V, Y, I, W, R, K, Q, A, D, S, or D;
X₁₁ comprises a residue selected from F, G, V, N, T, I, R, E, or A;
X₁₂ comprises a residue selected from I, S, R, V, P, G, T, L, M, F, or Y;
X₁₃ comprises a residue selected from V, I, F, D, P, H, A, V, or L;
X₁₄ comprises a residue selected from L, Q, R, E, P, K, H, W, F, or V;
X₁₅ comprises a residue selected from A, E, L, K, V, D, Y, R, or Q; and
X₁₆ comprises a residue selected from L or I, and
wherein the engineered IL2Rβ binding region 2 motif does not comprise SEQ ID NO:2 and the engineered IL2 polypeptide binds to IL2Rβ at a reduced affinity compared to a wild-type IL2.

2. The engineered IL2 polypeptide of claim 1, wherein the engineered IL2Rβ binding region 2 is selected from a group comprising or consisting of: RFKALIIEINFIVQLL (SEQ ID NO:4), RSRQLISNMNGIILKL (SEQ ID NO:5), RLTHLRNVIGVILVQL (SEQ ID NO:6), LSLREPIGNIVTSVRE (SEQ ID NO:7), NRTDLVGDVNATIKAL (SEQ ID NO:8), RNKGILGDISNIVLAL (SEQ ID NO:9), RSREVVSRIDAIILEL (SEQ ID NO:10), RPRGLISDISNIVLAL (SEQ ID NO:11), RPRGLIGNISNIVLAL (SEQ ID NO:12), RPRGLIGDINNIVLAL (SEQ ID NO:13), RPKGLISNISNIVLAL (SEQ ID NO:14), RPKGLISDINNIVLAL (SEQ ID NO:15), RPKGLIGNINNIVLAL (SEQ ID NO:16), RNRGLISNISNIVLAL (SEQ ID NO:17), RNRGLISDINNIVLAL (SEQ ID NO:18), RNRGLIGNINNIVLAL (SEQ ID NO:19), RNKGLISNINNIVLAL (SEQ ID NO:20), RPRGLIGDISNIVLAL (SEQ ID NO:21), RPKGLISDISNIVLAL (SEQ ID NO:22), RPKGLIGNISNIVLAL (SEQ ID NO:23), RPKGLIGDINNIVLAL (SEQ ID NO:24), RNRGLISDISNIVLAL (SEQ ID NO:25), RNRGLIGNISNIVLAL (SEQ ID NO:26), RNRGLIGDINNIVLAL (SEQ ID NO:27), RNKGLISNISNIVLAL (SEQ ID NO:28), RNKGLISDINNIVLAL (SEQ ID NO:29), RNKGLIGNINNIVLAL (SEQ ID NO:30), RPRDLISDISNIVLAL (SEQ ID NO:31), RPRGLISDINNIVLAL (SEQ ID NO:32), RPRGLISDISVIVLAL (SEQ ID NO:33), RPRGLISDISNIVLEL (SEQ ID NO:34), RPRDLISDINNIVLAL (SEQ ID NO:35), RPRDLISDISVIVLAL (SEQ ID NO:36), RPRDLISDISNIVLEL (SEQ ID NO:37), RPRGLISDINVIVLAL (SEQ ID NO:38), RPRGLISDINNIVLEL (SEQ ID NO:39), RPRGLISDISVIVLEL (SEQ ID NO:40), RPKDLISNISNIVLAL (SEQ ID NO:41), RPKGLISNINNIVLAL (SEQ ID NO:42), RPKGLISNISVIVLAL (SEQ ID NO:43), RPKGLISNISNIVLEL (SEQ ID NO:44), RPKGLISNISVIVLEL (SEQ ID NO: 194), RPRGLISNISVIVLEL (SEQ ID NO:195), RPRDLISNISNIVLEL (SEQ ID NO: 196), RPKGLISNINNIVLEL (SEQ ID NO: 197), RPKGLISDINNIVLEL (SEQ ID NO: 198), RPRDLISRIDAIVLEL (SEQ ID NO: 199), RNRGLIGNINNIVLEL (SEQ ID NO: 200), RPKGLISEINNIVLEL (SEQ ID NO: 201), and RPKGLISRINNIVLEL (SEQ ID NO: 202).

3. The engineered IL2 polypeptide of claim 1 or 2, wherein the engineered IL2Rβ binding region 2 is selected from a group comprising or consisting of: RFKALIIEINFIVQLL (SEQ ID NO:4), RSRQLISNMNGIILKL (SEQ ID NO:5), RLTHLRNVIGVILVQL (SEQ ID NO:6), RNKGILGDISNIVLAL (SEQ ID NO:9), RSREVVSRIDAIILEL (SEQ ID NO:10), RPRGLISDISNIVLAL (SEQ ID NO:11), RPRGLIGDINNIVLAL (SEQ ID NO:13), RPKGLISNISNIVLAL (SEQ ID NO:14), RPRGLIGDISNIVLAL (SEQ ID NO:21), RPKGLISDISNIVLAL (SEQ ID NO:22), RPKGLIGDINNIVLAL (SEQ ID NO:24), RNKGLISNISNIVLAL (SEQ ID NO:28), RNKGLISDINNIVLAL (SEQ ID NO:29), RPKGLISNISVIVLEL (SEQ ID NO: 194), RPRGLISNISVIVLEL (SEQ ID NO:195), RPRDLISNISNIVLEL (SEQ ID NO: 196), RPKGLISNINNIVLEL (SEQ ID NO: 197), RPKGLISDINNIVLEL (SEQ ID NO: 198), RPRDLISRIDAIVLEL (SEQ ID NO: 199), RNRGLIGNINNIVLEL (SEQ ID NO: 200), RPKGLISEINNIVLEL (SEQ ID NO: 201), and RPKGLISRINNIVLEL (SEQ ID NO: 202).

4. The engineered IL2 polypeptide of any one of claims 1-3, wherein the engineered IL2 polypeptide has at least a 2-fold decrease in affinity for IL2Rβ as compared to the wild-type IL2 or an IL2 having the sequence of SEQ ID NO:171.

5. The engineered IL2 polypeptide of any one of claims 1-4, wherein the engineered IL2 polypeptide has a decrease in affinity for IL2Rα as compared to wild-type IL2 or an IL2 having the sequence of SEQ ID NO:171.

6. The engineered IL2 polypeptide of any one of claims 1-4, wherein the engineered IL2 polypeptide has a similar affinity for IL2Rα as compared to wild-type IL2 or an IL2 having the sequence of SEQ ID NO:171.

7. The engineered IL2 polypeptide of any one of claims 1-6, comprising an engineered IL2 receptor α (IL2Rα) binding region 1 comprising at least one substitution at positions selected from K35, R38, F42, and Y45, wherein:
i) the substitution at position K35 is selected from: K35G, K35L, K35S, K35V, K35D, K35E, and K35C;
ii) the substitution at position R38 is selected from: R38V, R38D, R38E, R38S, R38I, R38A, R38Y, R38G, R38C, or R38N;
iii) the substitution at position F42 is selected from: F42A, F42R, F42G, F42I, F42L, F42P and F42H; and
iv) the substitution at position Y45 is Y45S, Y45P, Y45A, Y45V, Y45C, Y45T, and Y45F.

8. The engineered IL2 polypeptide of claim 7, wherein the substitution is at least 2, at least 3, or all 4 of positions K35, R38, F42, and Y45.

9. The engineered IL2 polypeptide of claim 7, wherein the substitution is K35L, R38D, and F42R

10. The engineered IL2 polypeptide of any of claims 1-9, wherein the engineered IL2 polypeptide binds to IL2Rα with at least 10-fold reduced binding kinetics as compared to wild-type IL2 or an IL2 having the sequence of SEQ ID NO:171.

11. An engineered interleukin-2 (IL2) polypeptide comprising a polypeptide having at least 90% sequence identity to SEQ ID NO:45
(APASSSTKKTQLQLEHLLLDLQMILNGINNYKNPLLTDMLTRKFY
MPKKATELKHLQCLEEELKPLEEVLNLAQSKNFHL-X₁-X₂-X₃-X₄-
X-₅-X₆-X₇-X₈-X₉-X₁₀-X₁₁-X₁₂-X₁₃-X₁₄-X₁₅-X₁₆-KGSETTFMC
EYADETATIVEFLNRWITFSQSIISTLT)
X₁ comprises a residue selected from R, S, L, N, F, K, or T;
X₂ comprises a residue selected from A, F, S, L, R, T, I, H, P, or N;
X₃ comprises a residue selected from K, R, T, S, I, or P;
X₄ comprises a residue selected from G, D, R, A, Q, H, N, Y, or E;
X₅ comprises a residue selected from I, P, T, S, K, F, V, or L;
X₆ comprises a residue selected from I, R, V, M, T, or L;
X₇ comprises a residue selected from A, R, M, I, S, N, G, or S;
X₈ comprises a residue selected from E, N, H, T, K, Y, S, L, V, D, or R;
X₉ comprises a residue selected from I, V, A, T, L, T, or M;
X₁₀ comprises a residue selected from N, G, V, Y, I, W, R, K, Q, A, D, S, or D;
X₁₁ comprises a residue selected from F, G, V, N, T, I, R, E, or A;
X₁₂ comprises a residue selected from I, S, R, V, P, G, T, L, M, F, or Y;
X₁₃ comprises a residue selected from V, I, F, D, P, H, A, V, or L;
X₁₄ comprises a residue selected from L, Q, R, E, P, K, H, W, F, or V;
X₁₅ comprises a residue selected from A, E, L, K, V, D, Y, R, or Q; and
X₁₆ comprises a residue selected from L or I, and
wherein the engineered IL2Rβ binding region 2 motif does not comprise SEQ ID NO:2 and the engineered IL2 polypeptide binds to IL2Rβ at a reduced affinity compared to a wild-type IL2.

12. The engineered IL2 polypeptide of claim 11, wherein the IL2Rβ binding region 2 is a sequence selected from a group comprising or consisting of: RFKALIIEINFIVQLL (SEQ ID NO:4), RSRQLISNMNGIILKL (SEQ ID NO:5), RLTHLRNVIGVILVQL (SEQ ID NO:6), LSLREPIGNIVTSVRE (SEQ ID NO:7), NRTDLVGDVNATIKAL (SEQ ID NO:8), RNKGILGDISNIVLAL (SEQ ID NO:9), RSREVVSRIDAIILEL (SEQ ID NO:10), RPRGLISDISNIVLAL (SEQ ID NO:11), RPRGLIGNISNIVLAL (SEQ ID NO:12), RPRGLIGDINNIVLAL (SEQ ID NO:13), RPKGLISNISNIVLAL (SEQ ID NO:14), RPKGLISDINNIVLAL (SEQ ID NO:15), RPKGLIGNINNIVLAL (SEQ ID NO:16), RNRGLISNISNIVLAL (SEQ ID NO:17), RNRGLISDINNIVLAL (SEQ ID NO:18), RNRGLIGNINNIVLAL (SEQ ID NO:19), RNKGLISNINNIVLAL (SEQ ID NO:20), RPRGLIGDISNIVLAL (SEQ ID NO:21), RPKGLISDISNIVLAL (SEQ ID NO:22), RPKGLIGNISNIVLAL (SEQ ID NO:23), RPKGLIGDINNIVLAL (SEQ ID NO:24), RNRGLISDISNIVLAL (SEQ ID NO:25), RNRGLIGNISNIVLAL (SEQ ID NO:26), RNRGLIGDINNIVLAL (SEQ ID NO:27), RNKGLISNISNIVLAL (SEQ ID NO:28), RNKGLISDINNIVLAL (SEQ ID NO:29), RNKGLIGNINNIVLAL (SEQ ID NO:30), RPRDLISDISNIVLAL (SEQ ID NO:31), RPRGLISDINNIVLAL (SEQ ID NO:32), RPRGLISDISVIVLAL (SEQ ID NO:33), RPRGLISDISNIVLEL (SEQ ID NO:34), RPRDLISDINNIVLAL (SEQ ID NO:35), RPRDLISDISVIVLAL (SEQ ID NO:36), RPRDLISDISNIVLEL (SEQ ID NO:37), RPRGLISDINVIVLAL (SEQ ID NO:38), RPRGLISDINNIVLEL (SEQ ID NO:39), RPRGLISDISVIVLEL (SEQ ID NO:40), RPKDLISNISNIVLAL (SEQ ID NO:41), RPKGLISNINNIVLAL (SEQ ID NO:42), RPKGLISNISVIVLAL (SEQ ID NO:43), RPKGLISNISNIVLEL (SEQ ID NO:44), RPKGLISNISVIVLEL (SEQ ID NO: 194), RPRGLISNISVIVLEL (SEQ ID NO:195), RPRDLISNISNIVLEL (SEQ ID NO: 196), RPKGLISNINNIVLEL (SEQ ID NO: 197), RPKGLISDINNIVLEL (SEQ ID NO: 198), RPRDLISRIDAIVLEL (SEQ ID NO: 199), RNRGLIGNINNIVLEL (SEQ ID NO: 200), RPKGLISEINNIVLEL (SEQ ID NO: 201), and RPKGLISRINNIVLEL (SEQ ID NO: 202).

13. The engineered IL2 polypeptide of claim 11, wherein the IL2Rβ binding region 2 is a sequence selected from a group comprising or consisting of: RFKALIIEINFIVQLL (SEQ ID NO:4), RSRQLISNMNGIILKL (SEQ ID NO:5), RLTHLRNVIGVILVQL (SEQ ID NO:6), RNKGILGDISNIVLAL (SEQ ID NO:9), RSREVVSRIDAIILEL (SEQ ID NO:10), RPRGLISDISNIVLAL (SEQ ID NO:11), RPRGLIGDINNIVLAL (SEQ ID NO:13), RPKGLISNISNIVLAL (SEQ ID NO:14), RPRGLIGDISNIVLAL (SEQ ID NO:21), RPKGLISDISNIVLAL (SEQ ID NO:22), RPKGLIGDINNIVLAL (SEQ ID NO:24), RNKGLISNISNIVLAL (SEQ ID NO:28), RNKGLISDINNIVLAL (SEQ ID NO:29), RPKGLISNISVIVLEL (SEQ ID NO: 194), RPRGLISNISVIVLEL (SEQ ID NO:195), RPRDLISNISNIVLEL (SEQ ID NO: 196), RPKGLISNINNIVLEL (SEQ ID NO: 197), RPKGLISDINNIVLEL (SEQ ID NO: 198), RPRDLISRIDAIVLEL (SEQ ID NO: 199), RNRGLIGNINNIVLEL (SEQ ID NO: 200), RPKGLISEINNIVLEL (SEQ ID NO: 201), and RPKGLISRINNIVLEL (SEQ ID NO: 202).

14. The engineered IL2 polypeptide of any one of claims 1-13, comprising a sequence selected from a group comprising or consisting of: SEQ ID NOS:46-102, 147-169, and 203-211.

15. An engineered IL2 polypeptide, comprising a sequence have at least 90% sequence identity to a sequence selected from a group consisting of: SEQ ID NOS:46-102, 147-169, and 203-211.

16. An engineered IL2 polypeptide, comprising a sequence selected from a group comprising or consisting of: SEQ ID NOS:46-102, 147-169, and 203-211.

17. A fusion polypeptide comprising a first polypeptide sequence and a second polypeptide sequence, wherein the first polypeptide sequence comprises an engineered IL2 polypeptide of any one of claims 1-16.

18. The fusion polypeptide of claim 17, wherein the second polypeptide sequence of the fusion protein include a Fc domain, antibody, antigen binding moiety, cytokine, half-life extending molecule, tag or marker polypeptide, targeting domain, transport molecule, immunotoxin, NKG2D, linker sequence, PEGylation, chemically linked small molecule, nucleic acid, or any combination thereof.

19. The fusion polypeptide of claim 17 or 18, wherein the second polypeptide sequence comprises an antibody heavy chain constant region.

20. The fusion polypeptide of claim 19, wherein the antibody heavy chain constant region is human IgG heavy chain constant region.

21. The fusion polypeptide of claim 19 or 20, wherein the antibody heavy chain constant region is a human IgG1 heavy chain constant region.

22. The fusion polypeptide of any one of claims 19-21, wherein the antibody heavy chain constant region comprises an amino acid sequence at least 90% identical to SEQ ID NO:137.

23. The fusion polypeptide of any one of claims 19-22, wherein the antibody heavy chain constant region comprises, relative to SEQ ID NO:137, one or more mutations selected from L234A, L235A, P329G, Y349C, S354C, T366S, T366W, L368A, F405K, K409A and Y407V, numbered according to the EU numbering system.

24. The fusion polypeptide of any one of claims 19-23, wherein the antibody heavy chain constant region comprises an amino acid sequence of SEQ ID NO:123 or SEQ ID NO:138.

25. The fusion polypeptide of any one of claims 19-24, wherein the fusion protein comprises a sequence having at least 90% sequence identity to a sequence selected from SEQ ID NOS: 124-128 and 190-193.

26. A monovalent engineered IL2-Fc fusion polypeptide complex, comprising:
a. a first polypeptide comprising a fusion polypeptide of any one of claims 17-25, and
b. a second polypeptide that forms a dimer with the first protein.

27. The monovalent engineered IL2-Fc fusion polypeptide complex of claim 26, wherein the second polypeptide comprises a heavy chain constant region.

28. The monovalent engineered IL2-Fc fusion polypeptide complex of claim 26 or 27, wherein second polypeptide comprises the sequence of SEQ ID NO:123.

29. The monovalent engineered IL2-Fc fusion polypeptide complex of any one of claims 26-28, comprising first and second polypeptide having the sequences of:
a. SEQ ID NOS: 124 and 123;
b. SEQ ID NOS: 125 and 123;
c. SEQ ID NOS: 126 and 123;
d. SEQ ID NOS: 127 and 123;
e. SEQ ID NOS: 128 and 123;
f. SEQ ID NOS: 190 and 123;
g. SEQ ID NOS: 191 and 123;
h. SEQ ID NOS: 192 and 123; or
i. SEQ ID NOS: 193 and 123.

30. The fusion polypeptide of any one of claims 17-29, wherein the fusion polypeptide further comprises an antigen binding moiety.

31. The fusion polypeptide of claim 17, wherein the second polypeptide comprises an antigen binding moiety.

32. The fusion polypeptide of claim 31, wherein the antigen binding moiety comprises an immunoglobulin.

33. The fusion polypeptide of claim 31 or 32, wherein the antigen binding moiety comprises a Fab molecule, an scFv, a bi-specific T-cell engager, a diabody, a single domain antibody, or a VHH antibody (nanobody).

34. The fusion polypeptide of any one of claims 30-33, wherein the antigen binding moiety binds PD-L1, PD-1, CTLA-4, TIM3, LAG3, B7-H2, B7-H3, CD4, CD8, or a cellular marker.

35. A protein complex, comprising a first polypeptide that is a fusion polypeptide of any one of claims 17-34 and a second polypeptide comprising an antigen binding moiety.

36. The protein complex of claim 35, wherein the second polypeptide comprises at least one polypeptide having the sequences of SEQ ID NOS:103 and 114 or SEQ ID NOS: 103 and 172.

37. The protein complex of claim 36, comprising polypeptides having the sequences of:
a. SEQ ID NOS: 103, 114, and 124;
b. SEQ ID NOS: 103, 114, and 125;
c. SEQ ID NOS: 103, 114, and 126;
d. SEQ ID NOS: 103, 114, and 127;
e. SEQ ID NOS: 103, 114, and 128;
f. SEQ ID NOS: 103, 172, and 190;
g. SEQ ID NOS: 103, 172, and 191;
h. SEQ ID NOS: 103, 172, and 192; or
i. SEQ ID NOS: 103, 172, and 193.

38. A bifunctional fusion protein, comprising:
(a) an engineered IL2 polypeptide comprising a sequence of any one of claims 1-25; and
(b) an antigen-binding moiety.

39. The bifunctional fusion protein of claim 38, wherein the antigen-binding moiety comprises an immunoglobulin, Fab molecule, an scFv, a diabody, a single domain antibody, or a VHH antibody.

40. The bifunctional fusion protein of claim 38 or 39, wherein the antigen-binding moiety binds PD-L1, PD-1, CTLA-4, TIM3, LAG3, B7-H2, B7-H3, CD4, CD8, or a cellular marker.

41. The bifunctional fusion protein of any one of claims 38-40, wherein the antigen-binding moiety bind PD-L1 and comprises:
(i) a light chain comprising the sequence of SEQ ID NO: 103; and
(ii) a heavy chain comprising the sequence of SEQ ID NO: 104.

42. The bifunctional fusion protein of any one of claims 38-41, further comprising an antibody heavy chain constant region.

43. The bifunctional fusion protein of claim 42, wherein the antibody heavy chain constant region is a human IgG heavy chain constant region.

44. The bifunctional fusion protein of claim 43, wherein the antibody heavy chain constant region is a human IgG1 heavy chain constant region.

45. The bifunctional fusion protein of any one of claims 42-44, wherein the antibody heavy chain constant region comprises an amino acid sequence at least 90% identical to SEQ ID NO:137.

46. The bifunctional fusion protein of any one of claims 42-45, wherein the antibody heavy chain constant region comprises, relative to SEQ ID NO:137, one or more mutations selected from L234A, L235A, P329G, Y349C, S354C, T366S, T366W, L368A, F405K, K409A and Y407V, numbered according to the EU numbering system.

47. The bifunctional fusion protein of any one of claims 42-46, wherein the antibody heavy chain constant region comprises an amino acid sequence of SEQ ID NO:123 or SEQ ID NO:138.

48. The bifunctional fusion protein of any one of claims 42-47, comprising a sequence selected from the group comprising or consisting of SEQ ID NOS:106-113 and SEQ ID NOS:173-177.

49. The bifunctional fusion protein of any one of claims 42-48, further comprising light chain sequence comprising a polypeptide having the sequence of SEQ ID NO:103.

50. A protein complex, comprising the bifunctional fusion protein of any one of claims 42-49, and a second antigen binding moiety.

51. The protein complex of claim 50, wherein the second antigen binding moiety comprises at least one polypeptide having the sequences of SEQ ID NOS:103 and 104 or SEQ ID NOS:103 and 172.

52. The protein complex of claim 51, comprising polypeptides having the sequences of:
a. SEQ ID NOS:104, 106, and 103;
b. SEQ ID NOS:104, 107, and 103;
c. SEQ ID NOS:104, 108, and 103;
d. SEQ ID NOS:104, 109, and 103;
e. SEQ ID NOS:104, 110, and 103;
f. SEQ ID NOS:104, 111, and 103;
g. SEQ ID NOS:104, 112, and 103;
h. SEQ ID NOS:104, 113, and 103;
i. SEQ ID NOS:172, 173, and 103;
j. SEQ ID NOS:172, 174, and 103;
k. SEQ ID NOS:172, 175, and 103;
l. SEQ ID NOS:172, 176, and 103; or
m. SEQ ID NOS:172, 177, and 103.

53. The bifunctional fusion protein of claim 38, wherein the antigen-binding moiety binds PD-1.

54. A protein complex, comprising the bifunctional fusion protein of any one of claims 38-53, and a second antigen binding moiety.

55. The protein complex of claim 54, wherein the second antigen binding moiety binds PD-1.

56. A protein complex, comprising polypeptides having the sequences of:
a. SEQ ID NOS: 133, 134, and 135; or
b. SEQ ID NOS: 133, 134, and 136.

57. The fusion polypeptide of claim 18, wherein the half-life extending molecule comprises an Fc domain, human serum albumin (HSA), an HSA binding molecule, or transferrin.

58. The fusion polypeptide of claim 18, wherein the half-life extending molecule comprises poly-ethylene glycol (PEG) or polypropylene glycol (PPG).

59. The fusion polypeptide of claim 17 or 18, wherein the second polypeptide comprises interleukin 2, interleukin-15, interleukin-7, interleukin-10, or C-C motif chemokine ligand 19 (CCL19).

60. The fusion polypeptide of claim 17 or 18, wherein the second polypeptide comprises a ligand or a scaffold protein.

61. An isolated polynucleotide encoding at least one polypeptide of any one of claims 1-60.

62. An expression vector comprising the polynucleotide of claim 61.

63. A modified cell comprising the isolated polynucleotide of claim 61 or the expression vector of claim 62.

64. A pharmaceutical composition comprising an engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, a vector of claim 62, or a modified cell of claim 63, and a pharmaceutically acceptable carrier.

65. An engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, a vector of claim 62, or a modified cell of claim 63 or a pharmaceutical composition of claim 64, for use in a method of modulating an immune response in a subject in need thereof.

66. A method of modulating an immune response in a subject in need thereof, comprising administering an effective amount of an engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, a vector of claim 62, or a modified cell of claim 63 or a pharmaceutical composition of claim 64 to the subject.

67. The use or method of claim 65 or 66, wherein the modulating the immune response comprises at least one of: enhancing effector T cell activity, enhancing NK cell activity, and suppressing regulatory T cell activity.

68. An engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, a vector of claim 62, or a modified cell of claim 63 or a pharmaceutical composition of claim 64, for use in a method of treating a disease in a subject in need thereof.

69. A method of treating a disease in a subject in need thereof, comprising administering an effective amount of an engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, a vector of claim 62, or a modified cell of claim 63 or a pharmaceutical composition of claim 64 to the subject.

70. The use or method according to claim 68 or 69, wherein the disease comprises cancer or immunosuppression.

71. The use or method according to claim 70, wherein the cancer comprises hematological malignancies such as lymphoma, leukemia, multiple myeloma, and/or solid tumors such as breast cancer, pancreatic cancer, lung cancer, glioblastoma, renal cell carcinoma, head & neck cancer, liver cancer, gastric cancer, colon/colorectal cancer, renal cancer, bladder cancer, or melanoma.

72. The use or method according to claim 65-71, wherein the subject is treated with an additional therapeutic agent.

73. The use or method according to claim 72, wherein the additional therapeutic agent is a vaccine, gene therapy, cellular therapy, or any combination thereof.

74. The use or method according to claim 73, wherein the cellular therapy an immune cell expressing a chimeric antigen receptor, an immune cell expressing an engineered T cell receptor, a tumor infiltrating lymphocyte, or any combination thereof.

75. A cell culture medium comprising, engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, a vector of claim 62, or a modified cell of claim 63.

76. A method of culturing a cell, comprising incubating a cell with the culture medium of claim 75.

77. The method of claim 76, wherein the cell is in a blood sample.

78. The method of claim 76, wherein the cell is in sample comprising PBMCs.

79. The method of claim 76, wherein the cell is a T cell, a NK cell, a T regulatory cell, or any combination thereof.

80. The method of claim 79, wherein the T cell is a CD4+ T cell, a CD8+ T cell, a gamma delta (γδ) T cell, or any combination thereof.

81. The method of any one of claims 76-79, wherein the engineered IL2 polypeptide, fusion polypeptide, protein complex, or bifunctional fusion protein biases the growth, expansion, or persistence of a cell-type.

82. A transgenic immune cell comprising engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, or a vector of claim 62.

83. The transgenic immune cell of claim 82, wherein the immune cell is a CD4+ T cell, a CD8+ T cell, a γδ T cell, a NK cell, a T regulatory cell, or any combination thereof.

84. The transgenic immune cell of claim 82 or 83, wherein the immune cell further comprises a chimeric antigen receptor (CAR).

85. The transgenic immune cell of claim 84, wherein the CAR targets an antigen produced by a cancer cell.

86. The transgenic immune cell of any one of claims 82-85, wherein the engineered IL2 polypeptide, fusion polypeptide, protein complex, or bifunctional fusion protein is secreted by the transgenic immune cell.

87. The transgenic immune cell of any one of claims 82-85, wherein the engineered IL2 polypeptide, fusion polypeptide, protein complex, or bifunctional fusion protein comprises a transmembrane domain or cell-surface anchor molecule and is localized to the surface of the cell.

88. The transgenic immune cell of any one of claims 85-87, wherein the transgenic immune cell is an armored CAR T cell.

89. A transgenic immune cell of any one of claims 82-88, for use in a method of treating a disease in a subject in need thereof.

90. A method of treating a disease in a subject in need thereof, comprising administering an effective amount of a transgenic immune cell of any one of claims 82-88.

91. The use or method according to claim 89 or 90, wherein the disease comprises cancer or immunosuppression.

92. The use or method according to claim 91, wherein the cancer comprises hematological malignancies such as lymphoma, leukemia, multiple myeloma, and/or solid tumors such as breast cancer, pancreatic cancer, lung cancer, glioblastoma, renal cell carcinoma, head & neck cancer, liver cancer, gastric cancer, colon/colorectal cancer, renal cancer, bladder cancer, or melanoma.

93. A transgenic feeder cell comprising an engineered IL2 polypeptide of any one of claims 1-16, a fusion polypeptide of any one of claims 17-34 and 58-60, a protein complex of any one of claims 35-37 and 56-57, a bifunctional fusion protein of any one of claims 38-55, a polynucleotide of claim 61, or a vector of claim 62.

94. The transgenic feeder cell of claim 93, wherein the feeder cell is a K562 cell, a 3T3 cell, a fibroblast, or an antigen presenting cell.

95. A method of in vitro or ex vivo expansion of immune cells, comprising contacting a population of immune cells with an effective amount of an engineered IL2 polypeptide of any one of claims 1-16, a fusion polypeptide of any one of claims 17-34 and 58-60, a protein complex of any one of claims 35-37 and 56-7, or a bifunctional fusion protein of any one of claims 38-55 under conditions sufficient to promote expansion of the population of immune cells, and cultivating the population of immune cells for a time sufficient to at least double the number of immune cells.

96. The method of claim 95, wherein the conditions sufficient to promote expansion includes one or more agents or ligands that are capable of activating an intracellular signaling domain of a TCR complex.

97. The method of claim 96, wherein the agent capable of activating an intracellular signaling domain of a TCR complex is an anti-CD3 antibody or binding domain, an anti-CD28 antibody or binding domain, or a combination thereof.

98. The method of any one of claims 95-97, further comprising cultivating the population of immune cells with an IL-7, IL-15, IL-21, or any combination thereof.

99. The method of any one of claims 95-98, wherein the population of immune cells is cultivated in the presence of a population of feeder cells.

100. The method of claim 99, wherein the feeder cells comprise or consist essentially of the transgenic feeder cells of claim 93 or 94.
